SEQUENCE LISTING (52 sequences)

SEQ ID NO: 1 - protein, 126 aa, Artificial Sequence
  PAIR 1 VH
EVQLLESGGGLVQPGGSLRLSCAASGFTFSSYAMSWVRQAPGKGLEWVSGISAIDQSTYYADSVKGRFTISRDNSKNTLYLQMNSLRAEDTAVYYCARQKFMQLWGGGLRYPFGYWGQGTMVTVSS

SEQ ID NO: 2 - protein, 122 aa, Artificial Sequence
  PAIR 2 VH
EVQLVESGGGLVQPGGSLRLSCAASGFTFRSFAMSWVRQAPGKGLELVSDLRTSGGSTYYADSVKGRLTISRDNSKNTLYLQMNSLRAEDTAVYYCAKSHYSTSWFGGFDYWGQGTLVTVSS

SEQ ID NO: 3 - protein, 121 aa, Artificial Sequence
  PAIR 3 VH
QVQLQESGPGLVKPSETLSLTCTVSGGSISSYYWSWIRQPPGKGLELIGYIYYSGSTNYNPSLKSRVTISVDTSKNHFSLKLSSVTAADTAVYYCARSQYTSSWYGSFDIWGQGTMVTVSS

SEQ ID NO: 4 - protein, 122 aa, Artificial Sequence
  PAIR 4 VH
QVQLVQSGAEVKKPGASVKVSCKASGYTFNSYGISWVRQAPGQGLEWMGWISSHNGNSHYVQKFQGRVSMTTDTSTSTAYMELRSLRSDDTAVYYCARHSYTTSWYGGFDYWGQGTLVTVSS

SEQ ID NO: 5 - protein, 122 aa, Artificial Sequence
  PAIR 5 VH
EVQLVESGGGLVQPGGSLRLSCAASGFTFSSYALTWVRQAPGKGLEWVSFISGSGGRPFYADSVKGRFTISRDNSKNMLYLQMNSLRAEDTAIYYCAKSLYTTSWYGGFDSWGQGTLVTVSS

SEQ ID NO: 6 - protein, 122 aa, Artificial Sequence
  PAIR 6 VH
EVQLVESGGGLVQPGGSLRLSCAASGFTFSNYAMTWVRQAPGKGLEWVSTISGSGDNTYYADSVQGRFTISRGHSKNTLYLQMNSLRAEDTAVYYCAKPTYSRSWYGAFDFWGQGTMVTVSS

SEQ ID NO: 7 - protein, 122 aa, Artificial Sequence
  PAIR 7 VH
EVQLVESGGNLEQPGGSLRLSCTASGFTFSRSAMNWVRRAPGKGLEWVSGISGSGGRTYYADSVKGRFTISRDNSKNTLYLQMNSLSAEDTAAYYCAKDSYTTSWYGGMDVWGHGTTVTVSS

SEQ ID NO: 8 - protein, 116 aa, Artificial Sequence
  PAIR 8 VH
EVQLLESGGGLVQPGGSLRLSCAASGFTFSDYYMNWVRQAPGKGLEWVSSISRYSSYIYYADSVKGRFTISRDNSKNTLYLQMNSLRAEDTAVYYCARDIGGMDVWGQGTLVTVSS

SEQ ID NO: 9 - protein, 119 aa, Artificial Sequence
  PAIR 9 VH
EVQLLESGGGLVQPGGSLRLSCAASGFTFSNYYMHWVRQAPGKGLEWVSSISARSRYHYYADSVKGRFTISRDNSKNTLYLQMNSLRAEDTAVYYCARLATRHNAFDIWGQGTLVTVSS

SEQ ID NO: 10 - protein, 119 aa, Artificial Sequence
  PAIR 10 VH
EVQLLESGGGLVQPGGSLRLSCAASGFTFSNYYMHWVRQAPGKGLEWVSSISARSSYIYYADSVKGRFTISRDNSKNTLYLQMNSLRAEDTAVYYCARLATRNNAFDIWGQGTLVTVSS

SEQ ID NO: 11 - protein, 119 aa, Artificial Sequence
  PAIR 11 VH
EVQLLESGGGLVQPGGSLRLSCAASGFTFSRYYMHWVRQAPGKGLEWVSSISAQSSHIYYADSVEGRFTISRDNSKNTLYLQMNSLRAEDTAVYYCARLATRQNAFDIWGQGTLVTVSS

SEQ ID NO: 12 - protein, 119 aa, Artificial Sequence
  PAIR 12 VH
EVQLLESGGGLVQPGGSLRLSCAASGFTFSNYYMHWVRQAPGKGLEWVSSISARSSYLYYADSVKGRFTISRDNSKNTLYLQMNSLRAEDTAVYYCARLATRHVAFDIWGQGTLVTVSS

SEQ ID NO: 13 - protein, 139 aa, Artificial Sequence
  PAIR 13 VH
MRAWIFFLLCLAGRALAQVQLMQSGAEVKKPGASVKVSCKASGYTFTSYWMHWVRQAPGQGLEWMGTIYPRNSNTDYNQKFKARVTMTRDTSTSTVYMELSSLRSEDTAVYYCARPLYYYLTSPPTLFWGQGTLVTVSS

SEQ ID NO: 14 - protein, 123 aa, Artificial Sequence
  PAIR 14 VH
EVQLVETGGGLIQPGGSLRLSCAASGFTFSSYAMSWVRQAPGKGLEWVSAISGSGGSTYYADSVKGRFTISRDNSKNTLYLQMNSLRAEDTAVYYCARTLHGIRAAYDAFIIWGQGTLVTVSS

SEQ ID NO: 15 - protein, 123 aa, Artificial Sequence
  PAIR 15 VH
EVQLVETGGGLIQPGGSLRLSCAASGFTFSFYAMSWVRQAPGKGLEWVSAISGSGGSTYYADSVKGRFTISRDNSKNTLYLQMNSLRAEDTAVYYCARTLHGIRAAYDAFIIWGQGTLVTVSS

SEQ ID NO: 16 - protein, 123 aa, Artificial Sequence
  PAIR 16 VH
EVQLVETGGGLIQPGGSLRLSCAASGFTFSFYAMSWVRQAPGKGLEWVSAISGSGGSTYYADSVKGRFTISRDNSKNTLYLQMNSLRAEDTAVYYCARTIHGIRAAYDAFIIWGQGTLVTVSS

SEQ ID NO: 17 - protein, 121 aa, Artificial Sequence
  PAIR 17 VH
EVQLVESGGGLVQPGGSLRLSCAASGFTFSSYWMYWVRQAPGKGLEWVAAITPNAGEDYYPESVKGRFTISRDNAKNSLYLQMNSLRAEDTAVYYCARGHYYYTSYSLGYWGQGTLVTVSS

SEQ ID NO: 18 - protein, 119 aa, Artificial Sequence
  PAIR 18 VH
EVQLVESGGGLVQPGGSLRLSCAASGFTFSSFSMSWVRQAPGKGLEWVATISGGKTFTDYVDSVKGRFTISRDDSKNTLYLQMNSLRAEDTAVYYCTRANYGNWFFEVWGQGTLVTVSS

SEQ ID NO: 19 - protein, 106 aa, Artificial Sequence
  PAIR 1 VL
SYVLTQPPSVSVSPGQTASITCSGEGMGDKYAAWYQQKPGQSPVLVIYRDTKRPSGIPERFSGSNSGNTATLTISGTQAMDEADYYCGVIQDNTGVFGGGTKLTVL

SEQ ID NO: 20 - protein, 107 aa, Artificial Sequence
  PAIR 2 VL
DIQMTQSPSSVSASVGDRVTITCRASQGFSSWLAWYQQKPGKAPKLLIYAASSLQSGVPSRFSGSGSGTDFTLTITNLQPEDFATYYCQQANSFPLTFGGGTKVEIK

SEQ ID NO: 21 - protein, 107 aa, Artificial Sequence
  PAIR 3 VL
DIQMTQSPSSVSASVGDRVTITCRASQGISTWLAWFQQKPGKAPKLLIYAASTLQGGVPSRFSGSGSGPEFTLTISSLQPEDFATYYCQQANSFPWTFGQGTKVEIK

SEQ ID NO: 22 - protein, 107 aa, Artificial Sequence
  PAIR 4 VL
DIQMTQSPSSVSASVGDRVTITCRASQGFSSWLAWYQQKPGKAPQLLIYAASSLQSGVPSRFSGSGSGSDFTLTISSLQPEDFATYYCQQANSFPLTFGGGTKVEIK

SEQ ID NO: 23 - protein, 107 aa, Artificial Sequence
  PAIR 5 VL
DIQMTQSPSSVSASVGDRVTITCRASQGVVSWLAWYQQKPGKAPKLLIYAASSLQSGVPSRFSGSGSGTDFTLTISSLQPEDFATYYCQQSNSFPFTLGPGTKVDIK

SEQ ID NO: 24 - protein, 107 aa, Artificial Sequence
  PAIR 6 VL
DIQMTQSPSSVSASVGDRVTITCRASQGISSWLAWYQQKPGKAPQLLIYAASRLQSGVPSRFWGSGSGTDFTLTISSLQPEDFATYYCQQANNFPFTFGPGTKVDIK

SEQ ID NO: 25 - protein, 107 aa, Artificial Sequence
  PAIR 7 VL
DIQMTQSPSSVSASVGDRVTITCRASQGIFSWLAWYQQKPGKAPKLLIYAASSLQSGVPSRFSGSGSGTDFTLTISSLQPEDFAIYYCQQANSVPITFGQGTRLEIK

SEQ ID NO: 26 - protein, 109 aa, Artificial Sequence
  PAIR 8 VL
QSVLTQPPSASGTPGQRVTISCTGSSSNIGAVYDVHWYQQLPGTAPKLLIYRNNQRPSGVPDRFSGSKSGTSASLAISGLRSEDEADYYCQTYDSSRWVFGGGTKLTVL

SEQ ID NO: 27 - protein, 110 aa, Artificial Sequence
  PAIR 9 VL
QSVLTQPPSASGTPGQRVTISCSGSSSNIGNNAVSWYQQLPGTAPKLLIYASNMRVIGVPDRFSGSKSGTSASLAISGLRSEDEADYYCGAWDDSQKALVFGGGTKLTVL

SEQ ID NO: 28 - protein, 110 aa, Artificial Sequence
  PAIR 10 VL
QSVLTQPPSASGTPGQRVTISCSGSSSNIGRNAVNWYQQLPGTAPKLLIYASNMRVSGVPDRFSGSKSGTSASLAISGLRSEDEADYYCWAWDDSQKVGVFGGGTKLTVL

SEQ ID NO: 29 - protein, 110 aa, Artificial Sequence
  PAIR 11 VL
QSVLTQPPSASGTPGQRVTISCSGSSSNIGRNAVNWYQQLPGTAPKLLIYASNMRRSGVPDRFSGSKSGTSASLAISGLRSEDEADYYCSAWDDSQKVVVFGGGTKLTVL

SEQ ID NO: 30 - protein, 110 aa, Artificial Sequence
  PAIR 12 VL
QSVLTQPPSASGTPGQRVTISCSGSSSNIGNNAVNWYQQLPGTAPKLLIYASNMRRPGVPDRFSGSKSGTSASLAISGLRSEDEADYYCEAWDDSQKAVVFGGGTKLTVL

SEQ ID NO: 31 - protein, 125 aa, Artificial Sequence
  PAIR 13 VL
MRAWIFFLLCLAGRALADIQLTQSPSFLSASVGDRVTITCKASQDVGTAVAWYQQKPGKAPKLLIYWASTRHTGVPSRFSGSGSGTEFTLTISSLQPEDFATYYCQQAKTYPFTFGSGTKLEIKR

SEQ ID NO: 32 - protein, 108 aa, Artificial Sequence
  PAIR 14 VL
EIVLTQSPGTLSLSPGERATLSCRASQSVGINLSWYQQKPGQAPRLLIYGASHRATGIPDRFSGSGSGTDFTLTISRLEPEDFAVYYCHQYSQSPPFTFGGGTKVEIK

SEQ ID NO: 33 - protein, 108 aa, Artificial Sequence
  PAIR 15 VL
EIVLTQSPGTLSLSPGERATLSCRASQSVGINLSWYQQKPGQAPRLLIYGASHRLTGIPDRFSGSGSGTDFTLTISRLEPEDFAVYYCHQYSQPPPFTFGGGTKVEIK

SEQ ID NO: 34 - protein, 108 aa, Artificial Sequence; identical to SEQ ID NO: 33
  PAIR 16 VL

SEQ ID NO: 35 - protein, 106 aa, Artificial Sequence
  PAIR 17 VL
DIQMTQSPSSLSASVGDRVTITCKASQNINKHLDWYQQKPGKAPKLLIYFTNNLQTGVPSRFSGSGSGTDFTLTISSLQPEDFATYYCFQYNQGWTFGGGTKVEIK

SEQ ID NO: 36 - protein, 111 aa, Artificial Sequence
  PAIR 18 VL
EIVLTQSPATLSLSPGERATLSCRASESVAKYGLSLLNWFQQKPGQPPRLLIFAASNRGSGIPARFSGSGSGTDFTLTISSLEPEDFAVYYCQQSKEVPFTFGQGTKVEIK

SEQ ID NO: 37 - protein, 5 aa, Artificial Sequence
  PAIR 1 HCDR1
SYAMS

SEQ ID NO: 38 - protein, 17 aa, Artificial Sequence
  PAIR 1 HCDR2
GISAIDQSTYYADSVKG

SEQ ID NO: 39 - protein, 17 aa, Artificial Sequence
  PAIR 1 HCDR3
QKFMQLWGGGLRYPFGY

SEQ ID NO: 40 - protein, 11 aa, Artificial Sequence
  PAIR 1 LCDR1
SGEGMGDKYAA

SEQ ID NO: 41 - protein, 7 aa, Artificial Sequence
  PAIR 1 LCDR2
RDTKRPS

SEQ ID NO: 42 - protein, 9 aa, Artificial Sequence
  PAIR 1 LCDR3
GVIQDNTGV

SEQ ID NO: 43 - protein, 34 aa, artificial sequence
  N terminal His10/Avitag/Factor Xa protease cleavage site
MHHHHHHHHHHAAGLNDIFEAQKIEWHEAAIEGR

SEQ ID NO: 44 - protein, 159 aa, homo sapiens
SITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 45 - protein, 159 aa, artificial sequence
  IL33-16
SITGISPITEYLASLSTYNDQSITFALEDESYEIYVEDLKKDEKKDKVLLSYYESQHPSNESGDGVDGKMLMVTLSPTKDFWLHANNKEHSVELHKSEKPLPDQAFFVLHNMHSNSVSFESKTDPGVFIGVKDNHLALIKVDSSENLSTENILFKLSET

SEQ ID NO: 46 - protein, 15 aa, artificial sequence
  Avitag sequence motif
GLNDIFEAQKIEWHE

SEQ ID NO: 47 - DNA, 19 bp, artificial
  gRNA vector targeting RAGE exon 3
tgaggggattttccggtgc

SEQ ID NO: 48 - DNA, 19 bp, artificial sequence
  RAGE forward primer
gttgcagcctcccaacttc

SEQ ID NO: 49 - DNA, 20 bp, artificial sequence
  RAGE reverse primer
aatgaggccagtggaagtca

SEQ ID NO: 50 - protein, 328 aa, homo sapiens
MGFWILAILTILMYSTAAKFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRKNPSKECF

SEQ ID NO: 51 - protein, 564 aa, artificial sequence
  Human ST2S-huIgG1 Fc-His6
MPLLLLLPLLWAGALAKFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRKNPSKECFEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 52 - protein, 277 aa, artificial sequence
  His10/Avitag human ASGPR ECD
MPLLLLLPLLWAGALAHHHHHHHHHHGGSGLNDIFEAQKIEWHEGGSQNSQLQEELRGLRETFSNFTASTEAQVKGLSTQGGNVGRKMKSLESQLEKQQKDLSEDHSSLLLHVKQFVSDLRSLSCQMAALQGNGSERTCCPVNWVEHERSCYWFSRSGKAWADADNYCRLEDAHLVVVTSWEEQKFVQHHIGPVNTWMGLHDQNGPWKWVDGTDYETGFKNWRPEQPDDWYGHGLGGGEDCAHFTDDGRWNDDVCQRPYRWVCETELDKASQEPPLL

(19) United States
(12) Patent Application Publication
(10) Pub. No.: US 2022/0380450 A1
(43) Pub. Date: Dec. 1, 2022

(54) METHODS OF USING IL-33 ANTAGONISTS

(71) Applicant: MedImmune Limited, Cambridge (GB)

(72) Inventors: Rania DAGHER, Gaithersburg, MD (US); Kirsty HOUSLAY, San Francisco, CA (US); Mahboobe GHAEDI, Gaithersburg, MD (US); Sam STRICKSON, San Francisco, CA (US); Emma Suzanne COHEN, San Francisco, CA (US); Maria BELVISI, Gaithersburg, MD (US); Xavier ROMERO ROS, Cambridge (GB)

(21) Appl. No.: 17/755,605

(22) PCT Filed: Nov. 3, 2020
(86) PCT No.: PCT/EP2020/080841

§ 371 Date: May 3, 2022

(30) Foreign Application Priority Data
Nov. 4, 2019 (EP) 19206984.7

(51) Int. Cl. C07K 16/24; A61P 11/00

(52) U.S. Cl. CPC C07K 16/244; A61P 11/00; C07K 2317/565; C07K 2317/51; C07K 2317/515

(57) ABSTRACT

The present disclosure relates to an IL-33 antagonist for use in the prevention or treatment of abnormal epithelium physiology or EGFR-mediated diseases, and corresponding methods of prevention or treatment comprising administering an IL-33 antagonist to a patient in need thereof.

This application claims priority to European Patent Application Number 19206984.7, filed Nov. 4, 2019. The content of this application is incorporated herein by reference in its entirety.

### TECHNICAL FIELD

The present disclosure relates to an IL-33 antagonist for use in the prevention or treatment of abnormal epithelium physiology or EGFR-mediated diseases, and corresponding methods of prevention or treatment comprising administering an IL-33 antagonist to a patient in need thereof.

### BACKGROUND ART

Interleukin-33 (IL-33), also known as IL-1F11, is a member of the IL-1 family of cytokines. IL-33 is a 270 amino acid protein consisting of two domains: a homeodomain and a cytokine (IL-1 like) domain. The homeodomain contains a nuclear localisation signal (NLS). IL-33 is known to exist in different forms; a reduced form (redIL-33) and an oxidised form (oxIL-33). Previous studies have shown that the reduced form is rapidly oxidised under physiological conditions to form at least one disulphide bond in the oxidised form, and that the two forms likely have different binding patterns and effects.

It was previously discovered that the reduced form of IL-33 binds to ST2, and is in fact the only known ligand of the ST2 receptor expressed by Th2 cells and mast cells. Reduced IL-33 stimulates target cells by binding ST2 and subsequently activating NFκB and MAP kinase pathways leading to production of cytokines and chemokines such as IL-4, IL-5 and IL-13 for promoting inflammation. Soluble ST2 (sST2) is thought to be a decoy receptor that prevents reduced-IL-33 signalling.

More recently, it was found that the oxidised form of IL-33 also has physiological effects. It was discovered that oxidised IL-33 does not bind ST2, but instead binds to the receptor for advanced glycation end products (RAGE) and signals through this alternative pathway.

There has been significant interest in IL-33 as a therapeutic target predominantly due to the ability of what is now known as the reduced form to stimulate ST2 and result in potent inflammatory effects. However, there has been little research and interest in the oxidised IL-33 pathway as a therapeutic target, partly due to its later discovery, and due to the fact that RAGE has many ligands and its downstream interactions are not well understood.

Described in more detail herein, is at least one of these downstream RAGE interactions resulting from oxidised IL-33 stimulation. It has surprisingly been discovered that RAGE complexes with epithelial growth factor receptor (EGFR) as part of the oxidised IL-33 pathway. Reduced IL-33 is rapidly converted to oxidised IL-33 which then binds to RAGE and complexes with EGFR to stimulate EGFR activity. The surprising discovery of the involvement of EGFR is significant in that EGFR is a key therapeutic target for many diseases involving aspects of abnormal epithelium physiology.

As a result of this discovery, it is believed that antagonists that can bind to either form of IL-33 may effectively prevent signalling of oxidised IL-33. This may be either directly by binding to oxidised IL-33 itself, or indirectly by inhibiting conversion of reduced IL-33 to oxidised IL-33, both of which in turn will prevent stimulation of RAGE and stimulation of EGFR. This reduction in EGFR stimulation will have therapeutic benefits in any EGFR-mediated diseases, but particularly in conditions where EGFR is overstimulated.

EGFR is known to have various homeostatic effects on epithelium physiology. EGFR stimulation increases epithelial cell differentiation, increases epithelial cell migration and increases epithelium mucosal production. It is believed that the inhibition of EGFR-mediated signalling will treat or prevent disorders in which there is an abnormal epithelium physiology, such as, abnormal airway epithelium tissue remodeling or overproduction of mucus.

IL-33 has previously been associated with tissue remodeling in the airways (Li et al JACI, 2014 134: 1422-32; Vannella et al Sci Transl Med, 337ra65; Allinne et al JACI, 2019, 144: 1624-37). However, this has been thought to occur indirectly via a self-perpetuating amplification loop, whereby IL-33 signaling up-regulates the expression of both IL-33 and its cognate receptor ST2, leading to chronic ST2 axis signaling. It has not previously been established or suggested that IL-33 itself directly impacts airway epithelium biology, since the activity via ST2 is mediated by innate cells on which ST2 is expressed, such as macrophages and type 2 innate lymphoid cells.

As noted above, the disclosure is based on the discovery that IL-33 also acts directly via a different mechanism; the RAGE-EGFR pathway; to directly impact epithelium physiology. This new understanding is important because it can be used to widen the therapeutic applications of IL-33 antagonists to treat more diseases, more symptoms of diseases and more patients. A therapeutic opportunity to directly control and inhibit IL-33-mediated EGFR-mediated signalling by targeting IL-33 has not previously been realized.

The disclosure of the present application shows for the first time that the use of an IL-33 antagonist can directly impact impaired epithelium repair responses, decrease epithelial goblet cell differentiation and proliferation, decrease mucus production, and improve mucociliary movement in patients with abnormal epithelium physiology, such as those with COPD or bronchitis, via direct inhibition of RAGE/EGFR-mediated oxIL-33 activity. Therefore, the research presented herein supports therapeutic uses of IL-33 antagonists in the direct prevention or treatment of abnormal epithelium physiology, typically resulting from EGFR-mediated effects, and thereby present in EGFR-mediated diseases.

### SUMMARY OF THE DISCLOSURE

According to a first aspect, there is provided an IL-33 antagonist for use in the prevention or treatment of abnormal epithelium physiology by modulating or inhibiting a RAGE-EGFR mediated effect.

According to an alternative first aspect, there is provided a method of prevention or treatment of abnormal epithelium physiology in a patient comprising: administering an effective amount of an IL-33 antagonist to a patient in need thereof to modulate or inhibit a RAGE-EGFR mediated effect.

According to an alternative first aspect, there is provided use of an IL-33 antagonist in the manufacture of a medicament for the prevention or treatment of abnormal epithelium physiology.

According to a second aspect, there is provided an IL-33 antagonist for use in the prevention or treatment of an EGFR-mediated disease.

According to an alternative second aspect, there is provided a method of prevention or treatment of an EGFR-mediated disease in a patient comprising: administering an effective amount of an IL-33 antagonist to a patient in need thereof.

According to an alternative second aspect, there is provided use of an IL-33 antagonist in the manufacture of a medicament for the prevention or treatment of an EGFR-mediated disease.

According to a third aspect, there is provided an IL-33 antagonist for use in the prevention or treatment of a disease by improving epithelium physiology.

According to an alternative third aspect, there is provided a method of prevention or treatment of a respiratory disease by improving epithelium physiology in a patient comprising: administering an effective amount of an IL-33 antagonist to a patient in need thereof.

According to an alternative third aspect, there is provided use of an IL-33 antagonist in the manufacture of a medicament for the prevention or treatment of a respiratory disease by improving epithelium physiology.

According to a fourth aspect, there is provided an IL-33 antagonist for use in the prevention or treatment of a disease by inhibiting EGFR mediated effects.

According to an alternative fourth aspect, there is provided a method of prevention or treatment of a respiratory disease by inhibiting EGFR mediated effects in a patient comprising: administering an effective amount of an IL-33 antagonist to a patient in need thereof.

According to an alternative fourth aspect, there is provided use of an IL-33 antagonist in the manufacture of a medicament for the prevention or treatment of a respiratory disease by inhibiting EGFR mediated effects.

According to a further aspect, there is provided an IL-33 antagonist for use in the prevention or treatment of a disease by inhibiting IL-33 mediated EGFR signalling.

According to an alternative further aspect, there is provided a method of prevention or treatment of a disease by inhibiting IL-33 mediated EGFR signalling in a patient comprising: administering an effective amount of an IL-33 antagonist to a patient in need thereof.

According to an alternative further aspect, there is provided use of an IL-33 antagonist in the manufacture of a medicament for the prevention or treatment of a disease by inhibiting IL-33 mediated EGFR signalling.

Further features and embodiments of the above defined aspects are described hereinbelow in headed sections. Each section is combinable with any of the above mentioned aspects in any compatible combination.

### DETAILED DESCRIPTION

### Definitions

‘IL-33’ protein as employed herein refers to interleukin 33, in particular a mammalian interleukin 33 protein, for example human protein deposited with UniProt number 095760. However, it clear that this entity is not a single species but instead exists as reduced and oxidized forms. Given the rapid oxidation of the reduced form in vivo, for example in the period 5 minutes to 40 minutes, and in vitro, prior art references to IL-33 may actually be references to the oxidized form. Furthermore, commercial assays may not effectively discriminate between the reduced and oxidized forms. The terms “IL-33” and “IL-33 polypeptide” are used interchangeably. In certain embodiments, IL-33 is full length. In another embodiment, IL-33 is mature, truncated IL-33 (amino acids 112-270). Recent studies suggest full length IL-33 is active (Cayrol and Girard, Proc Natl Acad Sci USA 106(22): 9021-6 (2009); Hayakawa et al., Biochem Biophys Res Commun. 387(1):218-22 (2009); Talabot-Ayer et al, J Biol Chem. 284(29): 19420-6 (2009)). However, N-terminally processed or truncated IL-33 including but not limited to aa 72-270, 79-270, 95-270, 99-270, 107-270, 109-270, 111-270, 112-270 may have enhanced activity (Lefrancais 2012, 2014). In another embodiment, IL-33 may include a full length IL-33, a fragment thereof, or an IL-33 mutant or variant polypeptide, wherein the fragment of IL-33 or IL-33 variant polypeptide retains some or all functional properties of active IL-33.

‘Oxidized IL-33’ or ‘oxIL-33’ as employed herein refers to the form of the IL-33 that binds to RAGE, and triggers RAGE-EGFR mediated signalling. Oxidised IL-33 is a protein visible as a distinct band, for example by western blot analysis under non-reducing conditions, in particular with a mass 4 Da less than the corresponding reduced from. In particular, it refers to a protein with one or two disulphide bonds between the cysteines independently selected from cysteines 208, 227, 232 and 259. In one embodiment, oxidized IL-33 shows no binding to ST2.

‘Reduced IL-33’ or ‘redIL-33’ as employed herein refers to the form of the IL-33 that binds to ST2 and triggers ST2 mediated signalling. In particular cysteines 208, 227, 232 and 259 of the reduced form are not disulfide bonded. In one embodiment, reduced IL-33 shows no binding to RAGE.

It should be understood that references to “WT IL-33” or “IL-33” may refer to either the reduced or oxidised forms, or both, unless it is clear from the context within which it is used that one of the forms is meant.

‘Antigenically distinct forms of IL-33’ as employed herein refers to any form of IL-33 which can act as an antigen and be bound by an antibody or binding fragment thereof, typically in the context of the present disclosure this means oxidised IL-33, reduced IL-33 and reduced IL-33/sST2 complexes. ST2 mediated signalling/effects' as employed herein refers to the IL-33/ST2 system where reduced IL-33 recognition by ST2 promotes dimerization with IL-1RAcP on the cell surface and within the cell recruitment of receptor complex components MyD88, TRAF6 and IRAK1-4 to intracellular TIR domain. Thus ST2 dependent signalling/effects may be interrupted and attenuated by perturbing the interaction of IL-33 with ST2 or alternatively by interrupting the interaction with IL-1RAcP.

‘RAGE-EGFR mediated signalling/effects’ as employed herein refers to the oxidised IL-33/RAGE-EGFR system where oxidised IL-33 recognition by RAGE promotes complexing with EGFR within cell membranes. Thus RAGE-EGFR mediated signalling/effects may be interrupted and attenuated by perturbing the interaction of oxidised IL-33 with RAGE, or by interrupting the conversion of reduced IL-33 into oxidised IL-33.

‘Attenuates the activity of’ as employed herein refers to reducing or inhibiting the relevant activity or stopping the relevant activity. Generally attenuation and inhibition are employed interchangeably herein.

It is to be noted that the term “a” or “an” entity refers to one or more of that entity; for example, “an anti-IL-33 antibody” is understood to represent one or more anti-IL-33 antibodies. As such, the terms “a” (or “an”), “one or more,” and “at least one” can be used interchangeably herein.

As used herein, the terms “treat” or “treatment” refer to both therapeutic treatment and prophylactic or preventative measures, wherein the object is to prevent or slow down (lessen) an undesired physiological change or disorder. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired, except where the subject is defined as a ‘healthy subject’. Mammalian subjects include humans; domestic animals; farm animals; such as dogs, cats, guinea pigs, rabbits, rats, mice, horses, cattle, cows, and so on.

### IL-33 Antagonist

The present disclosure relates to medical uses of an IL-33 antagonist, in particular medical uses for the prevention or treatment of a disease by inhibiting IL-33 mediated EGFR signalling. In particular instances, the disclosure relates to the use of an IL-33 antagonist for the prevention or treatment of abnormal epithelium physiology, which may be found in EGFR-mediated diseases.

‘IL-33 antagonist’ as employed herein refers to any agent which attenuates IL-33 activity, for example, reduced IL-33 activity, oxidised IL-33 activity or the activity of both. Suitably the IL-33 antagonist is specific to reduced and/or oxidised IL-33. Suitably, the attenuation is by binding IL-33 in reduced or oxidised forms. Suitably, wherein the antagonist attenuates reduced IL-33 activity and oxidised IL-33 activity, the attenuation is by binding to IL-33 in reduced form (i.e. by binding to reduced IL-33).

Suitably, the IL-33 antagonist is a binding molecule or fragment thereof.

A “binding molecule” or “antigen binding molecule” of the present disclosure refers in its broadest sense to a molecule that specifically binds an antigenic determinant. Suitably, the binding molecule specifically binds to IL-33, in particular reduced IL-33 or oxidised IL-33.

Suitably, the binding molecule may be selected from: an antibody, an antigen-binding fragment thereof, an aptamer, at least one heavy or light chain CDR of a reference antibody molecule, and at least six CDRs from one or more reference antibody molecules.

Suitably, the IL-33 antagonist is an antibody or binding fragment thereof. Suitably, the IL-33 antagonist is an anti-IL-33 antibody or binding fragment thereof. Suitably, the anti-IL-33 antibody or binding fragment thereof specifically binds to IL-33, in particular reduced IL-33 or oxidised IL-33.

“Antibody” as employed herein refers to an immunoglobulin molecule as discussed below in more detail, in particular a full-length antibody or a molecule comprising a full-length antibody, for example a DVD-Ig molecule and the like.

A “binding fragment thereof” is interchangeable with “antigen binding fragment thereof” and refers to an epitope/antigen binding fragment of an antibody fragment, for example comprising a binding region, in particular comprising 6 CDRs, such as 3 CDRs in heavy variable region and 3 CDRs in light variable region.

Suitably, the antibody or binding fragment thereof is selected from: naturally-occurring, polyclonal, monoclonal, multispecific, mouse, human, humanized, primatized, or chimeric. Suitably, the antibody or binding fragment thereof may be an epitope-binding fragment, e.g., Fab′ and F(ab′)2, Fd, Fvs, single-chain Fvs (scFv), disulfide-linked Fvs (sdFv), fragments comprising either a VL or VH domain, or fragments produced by a Fab expression library. Suitably, the antibody or binding fragment thereof may be a minibody, a diabody, a triabody, a tetrabody, or a single chain antibody. Suitably, the antibody or binding fragment thereof is a monoclonal antibody. ScFv molecules are known in the art and are described, e.g., in U.S. Pat. No. 5,892,019.

Immunoglobulin or antibody molecules of the disclosure can be of any type (e.g., IgG, IgE, IgM, IgD, IgA, and IgY), class (e.g., IgG1, IgG2, IgG3, IgG4, IgA1, and IgA2, etc.), or subclass of immunoglobulin molecule.

Suitably the IL-33 antagonist inhibits the activity of oxidised IL-33, suitably by inhibiting the formation of oxidised IL-33. Suitably the IL-33 antagonist inhibits the conversion of reduced IL-33 into oxidised IL-33.

Suitably the IL-33 antagonist is a reduced IL-33 antagonist. In other words, the IL-33 antagonist attenuates the activity of reduced IL-33. Suitably, the attenuation is by binding to reduced IL-33. Suitably, by binding to reduced IL-33 said antagonist also inhibits/attenuates the activity of oxidised IL-33, by preventing its conversion to the oxidised IL-33 form

Suitably, the inhibition of the activity of oxidised IL-33 down-regulates or turns off RAGE dependent signalling and/or RAGE mediated effects. Suitably, the inhibition down-regulates or turns off RAGE-EGFR dependent signalling and/or RAGE-EGFR mediated effects. Suitably, the inhibition down-regulates or turns off EGFR dependent signalling. Suitably, the inhibition down-regulates or turns off EGFR mediated effects. In particular, it has been shown that IL33 antagonists that bind to reduced IL-33 can prevent binding of oxidised IL-33 to RAGE, thereby inhibiting RAGE-EGFR signalling.

Suitably, the inhibition of the activity of oxidised IL-33 down-regulates or prevents RAGE-EGFR complexing. Suitably the inhibition down-regulates or prevents EGFR activation, suitably RAGE mediated EGFR activation.

Suitably, the IL-33 antagonist has all of the inhibitory effects described above. Suitably, the reduced IL-33 antagonist has all of the inhibitory effects described above.

Suitably the IL-33 antagonist is a reduced IL-33 binding molecule or fragment thereof. Suitably the IL-33 antagonist is a reduced IL-33 antibody or binding fragment thereof, suitably an anti-reduced IL33 antibody or binding fragment thereof.

Suitably, the binding molecule or a fragment thereof specifically binds to redIL-33 with a binding affinity (Kd) of less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷M, 10⁻⁷ M, 5×10⁻⁸ M, 10⁻⁸ M, 5×10⁻⁹M, 10⁻⁹M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M. Suitably, the binding affinity to redIL-33 is less than 5×10⁻¹⁴M (i.e. 0.05 pM). Suitably, the binding affinity is as measured using Kinetic Exclusion Assays (KinExA) or BIACORE™, suitably using KinExA, using protocols such as those described in WO2016/156440 (see e.g., Example 11), which is hereby incorporated by reference in its entirety. Binding molecules that bind to redIL-33 with this binding affinity appear to bind tightly enough to redIL-33 to prevent dissociation of the binding molecule/redIL-33 complex within biologically relevant timescales. Without wishing to be bound by theory, this binding strength is thought to prevent release of the antigen prior to degradation of the antibody/antigen complex in vivo, such that redIL-33 is not released and cannot undergo conversion from redIL-33 to oxIL-33. Thus, when binding to redIL-33 with this binding affinity, the binding molecule can inhibit or attenuate the activity of oxIL-33 by preventing its formation, thereby inhibiting RAGE signalling.

Suitably, the binding molecule or a fragment thereof may specifically bind to redIL-33 with an on rate (k(on)) of greater than or equal to 10³ M⁻¹ sec⁻¹, 5×10³ M⁻¹ sec⁻¹, 10⁴ M⁻¹ sec⁻¹ or 5×10⁴ M⁻¹ sec⁻¹. For example, a binding molecule of the disclosure may bind to redIL-33 or a fragment or variant thereof with an on rate (k(on)) greater than or equal to 10⁻⁶ M⁻¹ sec⁻¹, 5×10⁵ M⁻¹ sec⁻¹, 10⁶ M⁻¹ sec⁻¹, or 5×10⁶ M⁻¹ sec⁻¹ or 10⁷ M⁻¹ sec⁻¹. Suitably, the k(on) rate is greater than or equal to 10⁷ M⁻¹ sec⁻¹.

Suitably, the binding molecule or a fragment thereof may specifically bind to redIL-33 with an off rate (k(off)) of less than or equal to 5×10⁻¹ sec⁻¹, 10⁻¹ sec⁻¹, 5×10⁻¹ sec⁻¹, 10⁻¹ sec⁻¹, 5×10⁻¹ sec⁻¹ or 10⁻³ sec⁻¹. For example, a binding molecule of the disclosure may be said to bind to redIL-33 or a fragment or variant thereof with an off rate (k(off)) less than or equal to 5×10⁻¹ sec⁻¹, 10⁻¹ sec⁻¹, 5×10⁻⁵ sec⁻¹, 10⁻⁵ sec⁻¹, 5×10⁻⁶ sec⁻¹, 10⁻⁶ sec⁻¹, 5×10⁻⁷ sec⁻¹ or 10⁻⁷ sec⁻¹. Suitably, the k(off) rate is less than or equal to 10⁻³ sec⁻¹. IL-33 is an alarmin cytokine released rapidly and in high concentrations in response to inflammatory stimuli. redIL-33 is converted to the oxidised approximately 5-45 mins after release into the extracellular environment. Thus, to prevent conversion of redIL-33 to oxIL-33, the binding molecules described herein may bind to redIL-33 with these k(on) and/or k(off) rates. Without wishing to be bound by theory, these k(on)/k(off) rates are thought to ensure that the binding molecule can bind rapidly to redIL-33 before it converts to oxIL-33, thereby reducing the formation of oxIL-33, thereby attenuating RAGE signaling, suitably RAGE/EGFR signaling, and thereby attenuating RAGE/EGFR-mediated effects.

Suitably, the IL-33 binding molecule may competitively inhibit binding of IL-33 to the binding molecule 33_640087-7B (as described in WO2016/156440). Suitably, WO2016/156440 describes that 33_640087-7B binds to redIL-33 with particularly high affinity and attenuates both ST-2 and RAGE-dependent IL-33 signalling. Thus, a binding molecule that competitively inhibits binding of IL-33 to the binding molecule 33_640087-7B is highly likely to inhibit both redIL-33 and oxIL-33 signalling and thus be particularly suitable for use in the methods described herein.

A binding molecule or fragment thereof is said to competitively inhibit binding of a reference antibody to a given epitope if it specifically binds to that epitope to the extent that it blocks, to some degree, binding of the reference antibody to the epitope. Competitive inhibition may be determined by any method known in the art, for example, solid phase assays such as competition ELISA assays, Dissociation-Enhanced Lanthanide Fluorescent Immunoassays (DELFIA®, Perkin Elmer), and radioligand binding assays. For example, the skilled person could determine whether a binding molecule or fragment thereof competes for binding to redIL-33 by using an in vitro competitive binding assay, such as a derivation of the HTRF assay described in example 1 of WO2016/156440, which is hereby incorporated by reference. For example, the skilled person could label a recombinant antibody of Table 1 with a donor fluorophore and mix multiple concentrations with fixed concentration samples of acceptor fluorophore labelled-redIL-33. Subsequently, the fluorescence resonance energy transfer between the donor and acceptor fluorophore within each sample can be measured to ascertain binding characteristics. To elucidate competitive binding molecules the skilled person could first mix various concentrations of a test binding molecule with a fixed concentration of the labelled antibody of Table 1. A reduction in the FRET signal when the mixture is incubated with labelled IL-33 in comparison with a labelled antibody-only positive control would indicate competitive binding to IL-33. A binding molecule or fragment thereof may be said to competitively inhibit binding of the reference antibody to a given epitope by at least 90%, at least 80%, at least 70%, at least 60%, or at least 50%.

In some embodiments, the binding molecule is selected from any of the following anti-IL-33 antibodies: 33_640087-7B (as described in WO2016/156440), ANB020 known as Etokimab (as described in WO2015/106080), 9675P (as described in US2014/0271658), A25-3H04 (as described in US2017/0283494), Ab43 (as described in WO2018/081075), IL33-158 (as described in US2018/0037644), 10C12.38.H6.87Y.581 lgG4 (as described in WO2016/077381) or binding fragments thereof, each of the documents being incorporated herein by reference. All of these antibodies are referenced in Table 1.

Suitably, the IL-33 antagonist is an antibody or antigen-binding fragment comprising the complementarity determining regions (CDRs) of a variable heavy domain (VH) and a variable light domain (VL) pair selected from Table 1. Pair 1 corresponds to the VH and VL domain sequences of 33_640087-7B described in WO2016/156440. Pairs 2-7 correspond to VH and VL domain sequences of antibodies described in US2014/0271658. Pairs 8-12 correspond to VH and VL domain sequences of antibodies described in US2017/0283494. Pair 13 corresponds to the VH and VL domain sequences of ANB020, described in WO2015/106080. Pairs 14-16 correspond to VH and VL domain sequences of antibodies described in WO2018/081075. Pair 17 corresponds to VH and VL domain sequences of IL33-158 described in US2018/0037644. Pair 18 corresponds to VH and VL domain sequences of 10C12.38.H6.87Y.581 lgG4 described in WO2016/077381.

TABLE 1
Exemplary anti-IL-33 antibody VH and VL pairs

| | | | | LCVR amino acid |
|---|---|---|---|---|
| Pair | SEQ ID NO: | HCVR amino acid sequence | SEQ ID NO: | sequence |
| 1 | SEQ ID NO: 1 | EVQLLESGGGLVQPGGSL | SEQ ID NO: | SYVLTQPPSVSVSPGQ |
| | | RLSCAASGFTFSSYAMS | 19 | TASITCSGEGMGDKYA |
| | | WVRQAPGKGLEWVSGIS | | AWYQQKPGQSPVLVI |
| | | AIDQSTYYADSVKGRFTI | | YRDTKRPSGIPERFSGS |
| | | SRDNSKNTLYLQMNSLR | | NSGNTATLTISGTQAM |
| | | AEDTAVYYCARQKFMQL | | DEADYYCGVIQDNTG |
| | | WGGGLRYPFGYWGQGT | | VFGGGTKLTVL |
| | | MVTVSS | | |
| 2 | SEQ ID NO: 2 | EVQLVESGGGLVQPGGS | SEQ ID NO: | DIQMTQSPSSVSASVG |
| | | LRLSCAASGFTFRSFAMS | 20 | DRVTITCRASQGFSSW |
| | | WVRQAPGKGLELVSDLR | | LAWYQQKPGKAPKLLI |
| | | TSGGSTYYADSVKGRLTI | | YAASSLQSGVPSRFSG |
| | | SRDNSKNTLYLQMNSLR | | SGSGTDFTLTITNLQPE |
| | | AEDTAVYYCAKSHYSTS | | |
| | | WFGGFDYWGQGTLVTV | | DFATYYCQQANSFPLT |
| | | SS | | FGGGTKVEIK |
| 3 | SEQ ID NO: 3 | QVQLQESGPGLVKPSETL | SEQ ID NO: | DIQMTQSPSSVSASVG |
| | | SLTCTVSGGSISSYYWSW | 21 | DRVTITCRASQGISTW |
| | | IRQPPGKGLELIGYIYYSG | | LAWFQQKPGKAPKLLI |
| | | STNYNPSLKSRVTISVDTS | | YAASTLQGGVPSRFSG |
| | | KNHFSLKLSSVTAADTA | | SGSGPEFTLTISSLQPE |
| | | VYYCARSQYTSSWYGSF | | DFATYYCQQANSFPW |
| | | DIWGQGTMVTVSS | | TFGQGTKVEIK |
| 4 | SEQ ID NO: 4 | QVQLVQSGAEVKKPGAS | SEQ ID NO: | DIQMTQSPSSVSASVG |
| | | VKVSCKASGYTFNSYGIS | 22 | DRVTITCRASQGFSSW |
| | | WVRQAPGQGLEWMGWI | | LAWYQQKPGKAPQLLI |
| | | SSHNGNSHYVQKFQGRV | | YAASSLQSGVPSRFSG |
| | | SMTTDTSTSTAYMELRSL | | SGSGSDFTLTISSLQPE |
| | | RSDDTAVYYCARHSYTT | | DFATYYCQQANSFPLT |
| | | SWYGGFDYWGQGTLVT | | FGGGTKVEIK |
| | | VSS | | |
| 5 | SEQ ID NO: 5 | EVQLVESGGGLVQPGGS | SEQ ID NO: | DIQMTQSPSSVSASVG |
| | | LRLSCAASGFTFSSYALT | 23 | DRVTITCRASQGVVSW |
| | | WVRQAPGKGLEWVSFIS | | LAWYQQKPGKAPKLLI |
| | | GSGGRPFYADSVKGRFTI | | YAASSLQSGVPSRFSG |
| | | SRDNSKNMLYLQMNSLR | | SGSGTDFTLTISSLQPE |
| | | AEDTAIYYCAKSLYTTS | | DFATYYCQQSNSFPFT |
| | | WYGGFDSWGQGTLVTV | | LGPGTKVDIK |
| | | SS | | |
| 6 | SEQ ID NO: 6 | EVQLVESGGGLVQPGGS | SEQ ID NO: | DIQMTQSPSSVSASVG |
| | | LRLSCAASGFTFSNYAMT | 24 | DRVTITCRASQGISSWL |
| | | WVRQAPGKGLEWVSFIS | | AWYQQKPGKAPQLLI |
| | | GSGDNTYYADSVQGRFTI | | YAASRLQSGVPSRFWG |
| | | SRGHSKNTLYLQMNSLR | | SGSGTDFTLTISSLQPE |
| | | AEDTAVYYCAKPTYSRS | | DFATYYCQQANNFPFT |
| | | WYGAFDFWGQGTMVTV | | FGPGTKVDIK |
| | | SS | | |
| 7 | SEQ ID NO: 7 | EVQLVESGGNLEQPGGSL | SEQ ID NO: | DIQMTQSPSSVSASVG |
| | | RLSCTASGFTFSRSAMN | 25 | DRVTITCRASQGIFSWL |
| | | WVRRAPGKGLEWVSGIS | | AWYQQKPGKAPKLLI |
| | | GSGGRTYYADSVKGRFTI | | YAASSLQSGVPSRFSG |
| | | SRDNSKNTLYLQMNSLS | | SGSGTDFTLTISSLQPE |
| | | AEDTAAYYCAKDSYTTS | | DFAIYYCQQANSVPITF |
| | | WYGGMDVWGHGTTVTV | | GQGTRLEIK |
| | | SS | | |
| 8 | SEQ ID NO: 8 | EVQLLESGGGLVQPGGSL | SEQ ID NO: | QSVLTQPPSASGTPGQ |
| | | RLSCAASGFTFSDYYMN | 26 | RVTISCTGSSSNIGAVY |
| | | WVRQAPGKGLEWVSSIS | | DVHWYQQLPGTAPKL |
| | | RYSSYIYYADSVKGRFTI | | LIYRNNQRPSGVPDRF |
| | | SRDNSKNTLYLQMNSLR | | SGSKSGTSASLAISGLR |
| | | AEDTAVYYCARDIGGMD | | SEDEADYYCQTYDSSR |
| | | VWGQGTLVTVSS | | WVFGGGTKLTVL |
| 9 | SEQ ID NO: 9 | EVQLLESGGGLVQPGGSL | SEQ ID NO: | QSVLTQPPSASGTPGQ |
| | | RLSCAASGFTFSNYYMH | 27 | RVTISCSGSSSNIGNNA |
| | | WVRQAPGKGLEWVSSIS | | VSWYQQLPGTAPKLLI |
| | | ARSRYHYYADSVKGRFTI | | YASNMRVIGVPDRFSG |
| | | SRDNSKNTLYLQMNSLR | | SKSGTSASLAISGLRSE |
| | | AEDTAVYYCARLATRHN | | DEADYYCGAWDDSQK |
| | | AFDIWGQGTLVTVSS | | ALVFGGGTKLTVL |
| 10 | SEQ ID NO: | EVQLLESGGGLVQPGGSL | SEQ ID NO: | QSVLTQPPSASGTPGQ |
| | 10 | RLSCAASGFTFSNYYMH | 28 | RVTISCSGSSSNIGRNA |
| | | WVRQAPGKGLEWVSSIS | | VNWYQQLPGTAPKLLI |
| | | ARSSYIYYADSVKGRFTI | | YASNMRVSGVPDRFS |
| | | SRDNSKNTLYLQMNSLR | | GSKSGTSASLAISGLRS |
| | | AEDTAVYYCARLATRNN | | EDEADYYCWAWDDS |
| | | AFDIWGQGTLVTVSS | | QKVGVFGGGTKLTVL |
| 11 | SEQ ID NO: | EVQLLESGGGLVQPGGSL | SEQ ID NO: | QSVLTQPPSASGTPGQ |
| | 11 | RLSCAASGFTFSRYYMH | 29 | RVTISCSGSSSNIGRNA |
| | | WVRQAPGKGLEWVSSIS | | VNWYQQLPGTAPKLLI |
| | | AQSSHIYYADSVEGRFTIS | | YASNMRRSGVPDRFSG |
| | | RDNSKNTLYLQMNSLRA | | SKSGTSASLAISGLRSE |
| | | EDTAVYYCARLATRQNA | | DEADYYCSAWDDSQK |
| | | FDIWGQGTLVTVSS | | WVFGGGTKLTVL |
| 12 | SEQ ID NO: | EVQLLESGGGLVQPGGSL | SEQ ID NO: | QSVLTQPPSASGTPGQ |
| | 12 | RLSCAASGFTFSNYYMH | 30 | RVTISCSGSSSNIGNNA |
| | | WVRQAPGKGLEWVSSIS | | VNWYQQLPGTAPKLLI |
| | | ARSSYLYYADSVKGRFTI | | YASNMRRPGVPDRFSG |
| | | SRDNSKNTLYLQMNSLR | | SKSGTSASLAISGLRSE |
| | | AEDTAVYYCARLATRHV | | DEADYYCEAWDDSQK |
| | | AFDIWGQGTLVTVSS | | AVVFGGGTKLTVL |
| 13 | SEQ ID NO: | MRAWIFFLLCLAGRALA | SEQ ID NO: | MRAWIFFLLCLAGRAL |
| | 13 | QVQLMQSGAEVKKPGAS | 31 | ADIQLTQSPSFLSASVG |
| | | VKVSCKASGYTFTSYWM | | DRVTITCKASQDVGTA |
| | | HWVRQAPGQGLEWMGT | | VAWYQQKPGKAPKLL |
| | | IYPRNSNTDYNQKFKAR | | IYWASTRHTGVPSRFS |
| | | VTMTRDTSTSTVYMELSS | | GSGSGTEFTLTISSLQP |
| | | LRSEDTAVYYCARPLYY | | EDFATYYCQQAKTYPF |
| | | YLTSPPTLFWGQGTLVTV | | TFGSGTKLEIKR |
| | | SS | | |
| 14 | SEQ ID NO: | EVQLVETGGGLIQPGGSL | SEQ ID NO: | EIVLTQSPGTLSLSPGE |
| | 14 | RLSCAASGFTFSSYAMS | 32 | RATLSCRASQSVGINLS |
| | | WVRQAPGKGLEWVSAIS | | WYQQKPGQAPRLLIY |
| | | GSGGSTYYADSVKGRFTI | | GASHRATGIPDRFSGS |
| | | SRDNSKNTLYLQMNSLR | | GSGTDFTLTISRLEPED |
| | | AEDTAVYYCARTLHGIR | | FAVYYCHQYSQSPPFT |
| | | AAYDAFIIWGQGTLVTVS | | FGGGTKVEIK |
| | | s | | |
| 15 | SEQ ID NO: | EVQLVETGGGLIQPGGSL | SEQ ID NO: | EIVLTQSPGTLSLSPGE |
| | 15 | RLSCAASGFTFSFYAMS | 33 | RATLSCRASQSVGINLS |
| | | WVRQAPGKGLEWVSAIS | | WYQQKPGQAPRLLIY |
| | | GSGGSTYYADSVKGRFTI | | GASHRLTGIPDRFSGSG |
| | | SRDNSKNTLYLQMNSLR | | SGTDFTLTISRLEPEDF |
| | | AEDTAVYYCARTLHGIR | | AVYYCHQYSQPPPFTF |
| | | AAYDAFIIWGQGTLVTVS | | GGGTKVEIK |
| | | S | | |
| 16 | SEQ ID NO: | EVQLVETGGGLIQPGGSL | SEQ ID NO: | EIVLTQSPGTLSLSPGE |
| | 16 | RLSCAASGFTFSFYAMS | 34 | RATLSCRASQSVGINLS |
| | | WVRQAPGKGLEWVSAIS | | WYQQKPGQAPRLLIY |
| | | GSGGSTYYADSVKGRFTI | | GASHRLTGIPDRFSGSG |
| | | SRDNSKNTLYLQMNSLR | | SGTDFTLTISRLEPEDF |
| | | AEDTAVYYCARTIHGIRA | | AVYYCHQYSQPPPFTF |
| | | AYDAFIIWGQGTLVTVSS | | GGGTKVEIK |
| 17 | SEQ ID NO: | EVQLVESGGGLVQPGGS | SEQ ID NO: | DIQMTQSPSSLSASVG |
| | 17 | LRLSCAASGFTFSSYWM | 35 | DRVTITCKASQNINKH |
| | | YWVRQAPGKGLEWVAA | | LDWYQQKPGKAPKLLI |
| | | ITPNAGEDYYPESVKGRF | | YFTNNLQTGVPSRFSG |
| | | TISRDNAKNSLYLQMNSL | | SGSGTDFTLTISSLQPE |
| | | RAEDTAVYYCARGHYY | | DFATYYCFQYNQGWT |
| | | YTSYSLGYWGQGTLVTV | | FGGGTKVEIK |
| | | SS | | |
| 18 | SEQ ID NO: | EVQLVESGGGLVQPGGS | SEQ ID NO: | EIVLTQSPATLSLSPGE |
| | 18 | LRLSCAASGFTFSSFSMS | 36 | RATLSCRASESVAKYG |
| | | WVRQAPGKGLEWVATIS | | LSLLNWFQQKPGQPPR |
| | | GGKTFTDYVDSVKGRFTI | | LLIFAASNRGSGIPARF |
| | | SRDDSKNTLYLQMNSLR | | SGSGSGTDFTLTISSLE |
| | | AEDTAVYYCTRANYGN | | PEDFAVYYCQQSKEVP |
| | | WFFEVWGQGTLVTVSS | | FTFGQGTKVEIK |

Suitably, the IL-33 antagonist is an antibody or antigen-binding fragment comprising the complementarity determining regions (CDRs) of the heavy chain variable region (HCVR) comprising the sequence of SEQ ID NO:1 and the complementarity determining regions (CDRs) of light chain variable region (LCVR) comprising the sequence of SEQ ID NO:19. These CDRs correspond to those derived from 33_640087-7B (as described in WO2016/156440), which binds reduced IL-33 and inhibits its conversion to oxidised IL-33. 33_640087-7B is described in full in WO2016/156440 which is incorporated by reference herein.

Suitably, the IL-33 antagonist is an antibody or antigen-binding fragment comprising the complementarity determining regions (CDRs) of the heavy chain variable region (HCVR) comprising the sequence of SEQ ID NO:7 and the complementarity determining regions (CDRs) of light chain variable region (LCVR) comprising the sequence of SEQ ID NO:25. These CDRs correspond to those derived from the antibody 9675P. 9675P is described in full in US2014/0271658 which is incorporated by reference herein.

Suitably, the IL-33 antagonist is an antibody or antigen-binding fragment comprising the complementarity determining regions (CDRs) of the heavy chain variable region (HCVR) comprising the sequence of SEQ ID NO:11 and the complementarity determining regions (CDRs) of light chain variable region (LCVR) comprising the sequence of SEQ ID NO:29. These CDRs correspond to those derived from the antibody A25-3H04. A25-3H04 is described in full in US2017/0283494 which is incorporated by reference herein.

Suitably, the IL-33 antagonist is an antibody or antigen-binding fragment comprising the complementarity determining regions (CDRs) of the heavy chain variable region (HCVR) comprising the sequence of SEQ ID NO:13 and the complementarity determining regions (CDRs) of light chain variable region (LCVR) comprising the sequence of SEQ ID NO:31. These CDRs correspond to those derived from the antibody ANB020. ANB020 is described in full in WO2015/106080 which is incorporated by reference herein.

Suitably, the IL-33 antagonist is an antibody or antigen-binding fragment comprising the complementarity determining regions (CDRs) of the heavy chain variable region (HCVR) comprising the sequence of SEQ ID NO:16 and the complementarity determining regions (CDRs) of light chain variable region (LCVR) comprising the sequence of SEQ ID NO:34. These CDRs correspond to those derived from the antibody Ab43. Ab43 is described in full in WO2018/081075 which is incorporated by reference herein.

Suitably, the IL-33 antagonist is an antibody or antigen-binding fragment comprising the complementarity determining regions (CDRs) of the heavy chain variable region (HCVR) comprising the sequence of SEQ ID NO:17 and the complementarity determining regions (CDRs) of light chain variable region (LCVR) comprising the sequence of SEQ ID NO:35. These CDRs correspond to those derived from the antibody IL33-158. IL33-158 is described in full in US2018/0037644 which is incorporated by reference herein.

Suitably, the IL-33 binding molecule is an antibody or antigen-binding fragment comprising the complementarity determining regions (CDRs) of the heavy chain variable region (HCVR) comprising the sequence of SEQ ID NO:18 and the complementarity determining regions (CDRs) of light chain variable region (LCVR) comprising the sequence of SEQ ID NO:36. These CDRs correspond to those derived from the antibody 10C12.38.H6.87Y.581 lgG4. 10C12.38.H6.87Y.581 lgG4 is described in full in WO2016/077381 which is incorporated by reference herein.

Suitably the skilled person knows of available methods in the art to identify CDRs within the heavy and light variable regions of an antibody or antigen-binding fragment thereof. Suitably the skilled person may conduct sequence-based annotation, for example. The regions between CDRs are generally highly conserved, and therefore, logic rules can be used to determine CDR location. The skilled person may use a set of sequence-based rules for conventional antibodies (Pantazes and Maranas, Protein Engineering, Design and Selection, 2010), alternatively or additionally he may refine the rules based on a multiple sequence alignment. Alternatively, the skilled person may compare the antibody sequences to a publicly available database operating on Kabat, Chothia or IMGT methods using the BLASTP command of BLAST+ to identify the most similar annotated sequence. Each of these methods has devised a unique residue numbering scheme according to which it numbers the hypervariable region residues and the beginning and ending of each of the six CDRs is then determined according to certain key positions. Upon alignment with the most similar annotated sequence, for example, the CDRs can be extrapolated from the annotated sequence to the non-annotated sequence, thereby identifying the CDRs. Suitable tools/databases are: the Kabat database, Kabatman, Scalinger, IMGT, Abnum for example.

Suitably, the IL-33 antagonist is an antibody or antigen-binding fragment comprising a variable heavy domain (VH) and variable light domain (VL) pair selected from Table 1.

Suitably, the IL33 antibody or antigen binding fragment thereof comprises a VH domain of the sequence of SEQ ID NO:1 and a VL domain of the sequence of SEQ ID NO:19.

Suitably, the IL33 antibody or antigen binding fragment thereof comprises a VH domain of the sequence of SEQ ID NO:7 and a VL domain of the sequence of SEQ ID NO:25.

Suitably, the IL33 antibody or antigen binding fragment thereof comprises a VH domain of the sequence of SEQ ID NO:11 and a VL domain of the sequence of SEQ ID NO:29.

Suitably, the IL33 antibody or antigen binding fragment thereof comprises a VH domain of the sequence of SEQ ID NO:13 and a VL domain of the sequence of SEQ ID NO:31.

Suitably, the IL33 antibody or antigen binding fragment thereof comprises a VH domain of the sequence of SEQ ID NO:16 and a VL domain of the sequence of SEQ ID NO: 34.

Suitably, the IL33 antibody or antigen binding fragment thereof comprises a VH domain of the sequence of SEQ ID NO:17 and a VL domain of the sequence of SEQ ID NO:35.

Suitably, therefore, the IL-33 antagonist is a binding molecule which may comprise 3 CDRs, for example in a heavy chain variable region independently selected from SEQ ID NO: 1, 7, 11, 13, 16, 17 and 18.

Suitably the IL-33 antagonist is a binding molecule which comprises 3 CDRs in a heavy chain variable region according to SEQ ID NO:1.

Suitably, the IL-33 antagonist is a binding molecule which may comprise 3 CDRs in a light chain variable region independently selected from SEQ ID NO: 19, 25, 29, 31, 34, 35 and 36.

Suitably, the IL-33 antagonist is a binding molecule which comprises 3 CDRs in a light chain variable region according to SEQ ID NO:19.

Suitably, therefore, the IL-33 antagonist is a binding molecule which may comprise 3 CDRs, for example in a heavy chain variable region independently selected from SEQ ID NO: 1, 7, 11, 13, 16, 17 and 18 and 3 CDRs, for example in a light chain variable region independently selected from SEQ ID NO: 19, 25, 29, 31, 34, 35 and 36.

Suitably, therefore the IL-33 antagonist is a binding molecule which comprises 3 CDRs in a heavy chain variable region according to SEQ ID NO:1, and 3 CDRs in a light chain variable region according to SEQ ID NO:19.

Suitably, therefore, the IL-33 antagonist is a binding molecule which may comprise a variable heavy domain (VH) and a variable light domain (VL) having VH CDRs 1-3 having the sequences SEQ ID NO: 37, 38 and 39, respectively, wherein one or more VHCDRs have 3 or fewer single amino acid substitutions, insertions and/or deletions.

Suitably, therefore, the IL-33 antagonist is a binding molecule comprising a VH domain which comprises VHCDRs 1-3 of SEQ ID NO: 37, SEQ ID NO: 38 and SEQ ID NO: 39, respectively.

Suitably, therefore, the IL-33 antagonist is a binding molecule comprising a VH domain which comprises VHCDRs 1-3 consisting of SEQ ID NO: 37, SEQ ID NO: 38 and SEQ ID NO: 39, respectively.

Suitably, therefore, the IL-33 antagonist is a binding molecule which may comprise a variable heavy domain (VH) and a variable light domain (VL) having VL CDRs 1-3 having the sequences of SEQ ID NO: 40, 41 and 42, respectively, wherein one or more VLCDRs have 3 or fewer single amino acid substitutions, insertions and/or deletions.

Suitably, therefore, the IL-33 antagonist is a binding molecule comprising a VL domain which comprises VLCDRs 1-3 of SEQ ID NO: 40, SEQ ID NO: 41 and SEQ ID NO: 42, respectively.

Suitably, therefore, the IL-33 antagonist is a binding molecule comprising a VL domain which comprises VLCDRs 1-3 consisting of SEQ ID NO: 40, SEQ ID NO: 41 and SEQ ID NO: 42, respectively.

Suitably, therefore, the IL-33 antagonist is a binding molecule which may comprise a VHCDR1 having the sequence of SEQ ID NO: 37, a VHCDR2 having the sequence of SEQ ID NO: 38, a VHCDR3 having the sequence of SEQ ID NO: 39, a VLCDR1 having the sequence of SEQ ID NO: 40, a VLCDR2 having the sequence of SEQ ID NO: 41, and a VLCDR3 having the sequence of SEQ ID NO: 42.

Suitably, therefore the IL-33 antagonist is an antibody or binding fragment thereof comprising a VH and VL, wherein the VH has an amino acid sequence at least 90%, for example 91, 92, 93, 94, 95, 96, 97, 98, 99 or 100% identical to a VH according to SEQ ID NO: 1, 7, 11, 13, 16, 17 and 18.

Suitably, therefore the IL-33 antagonist is an antibody or binding fragment thereof comprising a VH and VL, wherein the VH has an amino acid sequence at least 90%, for example 91, 92, 93, 94, 95, 96, 97, 98, 99 or 100% identical to a VH according to SEQ ID NO: 1.

Suitably, therefore the IL-33 antagonist is an antibody or binding fragment thereof comprising a VH and VL, wherein a VH disclosed above, has a sequence with 1, 2, 3 or 4 amino acids in the framework deleted, inserted and/or independently replaced with a different amino acid.

Suitably, therefore the IL-33 antagonist is an antibody or binding fragment thereof comprising a VH and VL, wherein the VL has an amino acid sequence at least 90%, for example 91, 92, 93, 94, 95, 96, 97, 98, 99 or 100% identical to a VL according to SEQ ID NO: 19, 25, 29, 31, 34, 35 and 36.

Suitably, therefore the IL-33 antagonist is an antibody or binding fragment thereof comprising a VH and VL, wherein the VL has an amino acid sequence at least 90%, for example 91, 92, 93, 94, 95, 96, 97, 98, 99 or 100% identical to a VL according to SEQ ID NO: 19.

Suitably, therefore the IL-33 antagonist is an antibody or binding fragment thereof comprising a VH and VL, wherein a VL disclosed above has a sequence with 1, 2, 3 or 4 amino acids in the framework independently deleted, inserted and/or replaced with a different amino acid.

Suitably, therefore the IL-33 antagonist is an antibody or binding fragment thereof comprising a VH and VL, wherein the VH has an amino acid sequence at least 90%, for example 91, 92, 93, 94, 95, 96, 97, 98, 99 or 100% identical to a VH according to SEQ ID NO: 1, 7, 11, 13, 16, 17 and 18, and VL has an amino acid sequence at least 90%, for example 91, 92, 93, 94, 95, 96, 97, 98, 99 or 100% identical to a VL according to SEQ ID NO: 19, 25, 29, 31, 34, 35 and 36.

Suitably, therefore the IL-33 antagonist is an antibody or binding fragment thereof comprising a VH and VL, wherein the VH has an amino acid sequence consisting of SEQ ID NO: 1, 7, 11, 13, 16, 17 and 18, and the VL has an amino acid sequence consisting of SEQ ID NO: 19, 25, 29, 31, 34, 35 and 36.

Suitably, therefore the IL-33 antagonist is an antibody or binding fragment thereof comprising a VH and VL, wherein the VH has an amino acid sequence consisting of SEQ ID NO: 1, and the VL has an amino acid sequence consisting of SEQ ID NO: 19.

### Compositions and Administration

The IL-33 antagonists in the medical uses and methods described herein may be administered to a patient in the form of a pharmaceutical composition.

Suitably, any references herein to ‘a/the IL-33 antagonist’ may also refer to a pharmaceutical composition comprising an/the IL-33 antagonist. Suitably the pharmaceutical composition may comprise one or more IL-33 antagonists.

Suitably the IL-33 antagonist may be administered in a pharmaceutically effective amount for the in vivo treatment of abnormal epithelium physiology, or EGFR-mediated diseases, or respiratory diseases as defined in the medical use and method of treatment aspects herein.

Suitably a ‘pharmaceutically effective amount’ or ‘therapeutically effective amount’ of an IL-33 antagonist shall be held to mean an amount sufficient to achieve effective binding to IL-33 and to achieve a benefit, e.g. to ameliorate symptoms of a disease or condition as recited in the medical uses/methods herein.

Suitably, the IL-33 antagonist or a pharmaceutical composition thereof may be administered to a human or other animal in accordance with the aforementioned methods of treatment/medical uses in an amount sufficient to produce a therapeutic effect.

Suitably, the IL-33 antagonist or a pharmaceutical composition thereof can be administered to such human or other animal in a conventional dosage form prepared by combining the IL-33 antagonist with a conventional pharmaceutically acceptable carrier or diluent according to known techniques.

It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of IL-33 antagonists may prove to be particularly effective.

The amount of IL-33 antagonist that may be combined with the carrier materials to produce a single dosage form will vary depending upon the subject treated and the particular mode of administration. Suitably, the pharmaceutical composition may be administered as a single dose, multiple doses or over an established period of time in an infusion. Suitably, dosage regimens also may be adjusted to provide the optimum desired response (e.g., a therapeutic or prophylactic response).

Suitably, the IL-33 antagonist will be formulated so as to facilitate administration and promote stability of the IL-33 antagonist.

Suitably, pharmaceutical compositions are formulated to comprise a pharmaceutically acceptable, non-toxic, sterile carrier such as physiological saline, non-toxic buffers, preservatives and the like.

Suitably the pharmaceutical composition may comprise pharmaceutically acceptable carriers, including, e.g., water, ion exchangers, alumina, aluminium stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

Suitably the pharmaceutical composition may include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include, e.g. water, alcoholic/aqueous solutions, emulsions or suspensions, including saline and buffered media.

Suitably pharmaceutically acceptable carriers include, but are not limited to, 0.01-0.1 M and preferably 0.05 M phosphate buffer or 0.8% saline. Other common parenteral carriers include sodium phosphate solutions, Ringer's dextrose, dextrose and sodium chloride, lactated Ringer's, or fixed oils. Intravenous carriers include fluid and nutrient replenishers, electrolyte replenishers, such as those based on Ringer's dextrose, and the like. Preservatives and other additives may also be present such as, for example, antimicrobials, antioxidants, chelating agents, and inert gases and the like.

Suitably, pharmaceutical compositions for injectable use may include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will be preserved against the contaminating action of microorganisms, such as bacteria and fungi. Suitably, the carrier can be a solvent or dispersion medium containing, for example, water, ethanol, polyol (e.g. glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Suitable formulations for use in the therapeutic methods disclosed herein are described in Remington's Pharmaceutical Sciences (Mack Publishing Co.) 16th ed. (1980).

Suitably, prevention of the action of microorganisms can be achieved by various antibacterial and antifungal agents, for example, parabens, chlorobutanol, phenol, ascorbic acid, thimerosal and the like. In many cases, it will be suitable to include isotonic agents, for example, sugars, polyalcohols, such as mannitol, sorbitol, or sodium chloride in the pharmaceutical composition. Prolonged absorption of the injectable compositions can be brought about by including in the composition an agent which delays absorption, for example, aluminum monostearate and gelatin.

Suitably, sterile injectable solutions can be prepared by incorporating an active compound (e.g., an IL-33 antagonist by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the methods of preparation may be vacuum drying and freeze-drying, which yields a powder of an active ingredient plus any additional desired ingredient from a previously sterile-filtered solution thereof.

Methods of administering the IL-33 antagonist or a pharmaceutical composition thereof to a subject in need thereof are well known to or are readily determined by those skilled in the art.

Suitably, the route of administration of the IL-33 antagonist or pharmaceutical composition thereof may be, for example, oral, parenteral, by inhalation or topical. Suitably, the term parenteral as used herein includes, e.g., intravenous, intraarterial, intraperitoneal, intramuscular, subcutaneous, rectal, or vaginal administration.

Suitably, the IL-33 antagonist or pharmaceutical composition thereof may be orally administered in an acceptable dosage form including, e.g., capsules, tablets, aqueous suspensions or solutions.

Suitably, the IL-33 antagonist or pharmaceutical composition thereof may be administered by nasal aerosol or inhalation. Such compositions may be prepared as solutions in saline, employing benzyl alcohol or other suitable preservatives, absorption promoters to enhance bioavailability, and/or other conventional solubilizing or dispersing agents.

Suitably, parenteral formulations may be a single bolus dose, an infusion or a loading bolus dose followed with a maintenance dose. These compositions may be administered at specific fixed or variable intervals, e.g., once a day, or on an “as needed” basis.

Suitably, the IL-33 antagonists or pharmaceutical compositions thereof are delivered directly to the site of the disease or condition, for example the abnormal epithelium physiology, thereby increasing the exposure of the diseased tissue to the therapeutic agent. Suitably, the IL-33 antagonists or pharmaceutical compositions thereof are administered directly to the site of the disease or condition. Suitably, therefore, the IL-33 antagonists or pharmaceutical compositions thereof are administered to the site of abnormal epithelium physiology, EGFR mediated disease, or respiratory disease.

In one embodiment, the administration of the IL-33 antagonist or pharmaceutical composition thereof is to the respiratory tract. Suitably by intranasal administration. Suitably by intranasal inhalation. Suitably, the IL-33 antagonist or pharmaceutical composition thereof may be provided in an inhaler device. Suitable inhaler devices are well known in the art.

In one embodiment there is provided an inhaler comprising an IL-33 antagonist or pharmaceutical composition thereof for use in the prevention or treatment of a condition or disease as defined herein.

Suitably, therefore, the IL-33 antagonist or pharmaceutical composition thereof is formulated as a liquid composition. Suitably as a liquid composition which can be aerosolized.

In one embodiment, the IL-33 antagonist or pharmaceutical composition thereof is provided as an aerosol.

Suitably, the components as recited hereinabove for preparing a pharmaceutical composition described herein may be packaged and sold in the form of a kit. Such a kit will suitably have labels or package inserts indicating that the associated pharmaceutical compositions are useful for treating a subject suffering from, or predisposed to a disease or disorder.

Suitably, the components for liquid formulations are processed, filled into containers such as ampoules, bags, bottles, syringes or vials, and sealed under aseptic conditions according to methods known in the art. Suitably the containers may be pressurised, suitably they may be aerosol containers. These containers may be included in a kit as described above. Suitably the kit may further comprise an inhaler device. Suitably the inhaler device comprises an IL-33 antagonist or pharmaceutical composition described herein, or is operable to comprise a container as described above which may comprise an IL-33 antagonist or pharmaceutical composition described herein.

### Abnormal Epithelium Physiology

The present disclosure relates to medical uses of IL-33 antagonist for the prevention or treatment of abnormal epithelium physiology.

“Abnormal epithelium physiology” as employed herein means any abnormality in the functioning of an epithelium in the human body. Functions of epithelium in the human body include: acting as a barrier to protect tissues beneath; regulation and exchange of chemical entities between tissues and a cavity; secretion of chemicals into a cavity; and sensation. Abnormalities in any of these functions can have devastating physiological effects. Epithelium is present in a wide range of tissues in the body including the skin, respiratory tract, gastrointestinal tract, reproductive tract, urinary tract, exocrine and endocrine glands, as such, abnormalities within the epithelium can be involved in a wide range of diseases or conditions. Suitably, the epithelium is the airway epithelium and abnormal epithelium physiology is abnormal airway epithelium physiology.

“Abnormal” as employed herein means a difference in a function compared with said function in a healthy subject, typically an increase or a decrease in a function compared with said function in a healthy subject.

Suitably the epithelium is selected from: squamous, cuboidal, columnar and pseudostratified. Suitably the epithelium is columnar.

Suitably the epithelium is ciliated. Suitably the epithelium is ciliated columnar. Suitably the abnormal epithelium physiology is abnormal ciliated columnar epithelium physiology.

Suitably, abnormal epithelium physiology includes abnormal epithelial cell migration. Suitably, abnormal epithelium physiology may include decreased epithelial cell migration. Suitably, abnormal epithelium physiology may include abnormal epithelial cell proliferation. Suitably, abnormal epithelium physiology may include decreased epithelial cell proliferation.

Suitably a decrease in epithelial cell migration leads to an impaired ability of the epithelium to repair wounds. Suitably, abnormal epithelium physiology includes impaired wound repair. Impaired wound repair may include impaired wound closure and reduced wound cell density.

Suitably treatment of abnormal epithelium physiology may comprise increasing or improving epithelial cell migration. Suitably treatment of abnormal epithelium physiology may comprise increasing or improving epithelium wound repair. Suitably treatment of abnormal epithelium physiology may comprise increasing or improving wound closure. Suitably treatment of abnormal epithelium physiology may comprise increasing or improving wound cell density.

Suitably the abnormal epithelium physiology is abnormal mucociliary physiology.

“Abnormal mucociliary physiology” as employed herein means any abnormality in the functioning of specifically the mucociliary roles of an epithelium. Abnormality in the functioning of the mucociliary roles of the epithelium may be due to abnormality in the functioning of the ciliated columnar cells and/or the goblet cells which are key to mucociliary functions. Suitably, the abnormal mucociliary physiology is due to abnormal functioning of the goblet cells.

“Mucociliary” as employed herein refers to the function of ciliated columnar cells and goblet columnar cells within an epithelium to secrete and move mucus. Mucociliary roles of an epithelium can include: proliferation of goblet cells; differentiation of goblet cells; secretion of mucus; regulation of mucus composition; and/or movement or clearance of mucus.

In one embodiment, there is provided an IL-33 antagonist for use in the prevention or treatment of abnormal mucociliary physiology, such as abnormal mucociliary physiology of an epithelium.

In one embodiment, there is provided a method of prevention or treatment of abnormal mucociliary physiology, such as abnormal mucociliary physiology of an epithelium, in a patient comprising: administering an effective amount of an IL-33 antagonist to a patient in need thereof.

Abnormal mucociliary physiology may include any abnormal function of the ciliated columnar cells or goblet cells of an epithelium. Suitably abnormal mucociliary physiology includes: abnormal mucus production; abnormal goblet cell differentiation; abnormal goblet cell proliferation; abnormal thickness of the epithelium; abnormal mucus clearance; and/or abnormal mucus composition.

Suitably abnormal mucus production comprises abnormal MUC5AC production. Suitably abnormal goblet cell differentiation comprises abnormal MUC5AC+goblet cell differentiation. Suitably abnormal goblet cell proliferation comprises abnormal MUC5AC+goblet cell proliferation. Suitably abnormal thickness of the epithelium comprises an abnormal amount of MUC5AC⁺ goblet cells in the total tissue area of the epithelium.

Suitably abnormal mucociliary physiology includes: increased goblet cell numbers; increased mucus production; increased goblet cell differentiation; increased thickness of the epithelium; and/or decreased mucus clearance.

Suitably increased mucus production comprises increased MUC5AC production. Suitably increased goblet cell differentiation comprises increased MUC5AC+goblet cell differentiation. Suitably increased goblet cell proliferation comprises increased MUC5AC+goblet cell proliferation. Suitably increased thickness of the epithelium comprises an increased amount of MUC5AC⁺ goblet cells in the total tissue area of the epithelium.

Suitably increased MUC5AC production is caused by an increase in MUC5AC gene expression. Suitably abnormal mucociliary physiology comprises increased MUC5AC gene expression in cells of the epithelium. Suitably, abnormal mucociliary physiology comprises increased expression of MUC5AC in goblet cells of the epithelium.

Suitably abnormal mucociliary physiology includes a change in mucus composition. Such a change may include an increased or a decrease in the ratio of the different mucus compounds contained in mucus, an increase or decrease in one or more specific mucus compounds, or an increase or decrease in the concentration or thickness of mucus.

Changes in mucus composition may comprise an increase or decrease in the ratio of different mucins, such as an increase or decrease in the ratio of mucins MUC5AC and MUC5B.

Changes in mucus composition may comprise an increase or decrease in the concentration of mucins. Suitably, changes in mucus composition comprise a decrease in the concentration of Mucin 5AC. Suitably, changes in mucus composition comprise a decrease in the number of goblet cells with upregulated MUC5AC expression.

Such changes in mucins contained in mucus may be measured and calculated as described in WO2018/204598 incorporated by reference herein.

Suitably abnormal mucus composition comprises an increase in the ratio of MUC5AC:MUC5B. Suitably abnormal mucus composition comprises an increase in MUC5AC contained in mucus. Suitably abnormal mucus composition comprises an increase in thickness of mucus.

Abnormal mucociliary physiology may comprise any one or more of the above symptoms in combination.

Suitably abnormal epithelium physiology includes abnormal tissue remodelling, such as abnormal epithelium remodelling. Suitably abnormal epithelium physiology includes increased tissue remodelling. Suitably abnormal epithelium physiology includes increased epithelium remodelling.

Abnormal epithelium physiology may comprise any one or more of the above symptoms in combination.

Treatment or prevention of abnormal epithelium physiology, or treatment or prevention of abnormal mucociliary physiology may comprise:
improving or increasing mucociliary clearance;
reducing or inhibiting mucus production;
inhibiting abnormal mucus composition;
reducing or inhibiting epithelium remodelling; and/or
reducing or inhibiting goblet cell differentiation and/or proliferation.

Suitably, reducing or inhibiting mucus production comprises reducing or inhibiting MUC5AC production. Suitably therefore, the treatment or prevention reduces or inhibits MUC5AC production.

Suitably inhibiting abnormal mucus composition may comprise restoring a normal mucus composition. Suitably this may comprise reducing the ratio of MUC5AC:MUC5B. Suitably therefore, the treatment or prevention reduces the ratio of MUC5AC:MUC5B. Suitably, the prevention or treatment inhibits or reduces MUC5AC in mucus. Suitably the prevention or treatment reduces the thickness of mucus.

Suitably, reducing or inhibiting goblet cell differentiation and/or proliferation comprises reducing or inhibiting MUC5AC⁺ goblet cell differentiation or proliferation. Suitably therefore, the treatment or prevention reduces or inhibits MUC5AC⁺ goblet cell differentiation or proliferation.

Suitably reducing or inhibiting epithelium remodeling comprises reducing the thickness of the respiratory epithelium. Suitably therefore, the treatment or prevention reduces the thickness of the respiratory epithelium.

Suitably reducing or inhibiting epithelium remodeling comprises reducing the amount of MUC5AC⁺ goblet cells in the total tissue area of the epithelium. Suitably therefore, the treatment or prevention reduces or inhibits the amount of MUC5AC⁺ goblet cells in the total tissue area of the epithelium.

Improving or increasing mucociliary clearance comprises improving or increasing mucociliary movement. Suitably therefore, the treatment or prevention improves or increases mucociliary movement.

Suitably the epithelium is respiratory epithelium. Suitably the abnormal epithelium physiology is abnormal epithelium physiology in respiratory epithelium.

In one embodiment, there is provided an IL-33 antagonist for use in the treatment of abnormal epithelium physiology in a respiratory disease.

In one embodiment, there is provided a method of prevention or treatment of abnormal epithelium physiology in a patient having a respiratory disease comprising: administering an effective amount of an IL-33 antagonist to a patient in need thereof.

Suitable respiratory diseases are defined elsewhere herein.

Suitably the abnormal epithelium physiology is abnormal mucociliary physiology in respiratory epithelium.

In one embodiment, there is provided an IL-33 antagonist for use in the prevention or treatment of abnormal mucociliary physiology in respiratory epithelium.

In one embodiment, there is provided a method of prevention or treatment of abnormal mucociliary physiology of the respiratory epithelium of a patient comprising: administering an effective amount of an IL-33 antagonist to a patient in need thereof.

Suitably the abnormal epithelium physiology is abnormal mucociliary physiology in a respiratory disease.

In one embodiment, there is provided an IL-33 antagonist for use in the prevention or treatment of abnormal mucociliary physiology in a respiratory disease.

In one embodiment, there is provided a method of prevention or treatment of abnormal mucociliary physiology in a patient having a respiratory disease comprising: administering an effective amount of an IL-33 antagonist to a patient in need thereof.

Suitably the abnormal epithelium physiology is present in the respiratory tract. Suitably the abnormal epithelium physiology is abnormal epithelium physiology of the respiratory tract. Suitably the abnormal epithelium physiology is abnormal mucociliary physiology of the respiratory tract.

The respiratory tract includes the upper and lower respiratory tract. Typically the upper respiratory tract includes the nasal passages, paranasal sinuses, pharynx and larynx. Typically the lower respiratory tract includes the trachea, bronchi, bronchioles, alveolar ducts, and the alveoli.

Suitably, the abnormal epithelium physiology is abnormal epithelium physiology of the lower respiratory tract, such as the bronchi.

Suitably the abnormal epithelium physiology is abnormal epithelium physiology of the lower respiratory tract. Suitably the abnormal epithelium physiology is abnormal epithelium physiology of the bronchi. Suitably, the abnormal lower respiratory tract epithelium physiology is abnormal mucociliary physiology of the lower respiratory tract. Suitably, abnormal mucociliary physiology of the lower respiratory tract is abnormal mucociliary physiology of the bronchi.

In one embodiment, there is provided an IL-33 antagonist for use in the prevention or treatment of abnormal mucociliary physiology of the lower respiratory tract.

In one embodiment, there is provided a method of prevention or treatment of abnormal mucociliary physiology of the lower respiratory tract of a patient, comprising: administering an effective amount of an IL-33 antagonist to a patient in need thereof.

In one embodiment, there is provided an IL-33 antagonist for use in the prevention or treatment of abnormal mucociliary physiology of the bronchi.

In one embodiment, there is provided a method of prevention or treatment of abnormal mucociliary physiology of the bronchi of a patient, comprising: administering an effective amount of an IL-33 antagonist to a patient in need thereof.

### EGFR Signalling

The present disclosure is based on the discovery that oxidised IL-33 binds to RAGE, which in turn complexes with EGFR and acts to disrupt epithelium homeostasis. The use of IL-33 antagonists can inhibit the signalling of oxidised IL-33 and thereby inhibit the activation of RAGE and inhibit RAGE-EGFR complexing. The data disclosed herein demonstrate that preventing the formation of RAGE-EGFR complexes prevents IL-33-mediated EGFR signalling, and restores normal epithelium physiology.

Suitably the IL-33 antagonist inhibits oxidised IL-33 signalling.

Suitably the IL-33 antagonist inhibits binding of oxidised IL-33 to RAGE.

Suitably the IL-33 antagonist inhibits the formation of RAGE-EGFR complexes. Suitably the IL-33 antagonist inhibits the formation of oxidised-IL33-RAGE-EGFR complexes.

Suitably the IL-33 antagonist inhibits clustering of EGFR. Suitably the IL-33 antagonist inhibits clustering of EGFR in the cell membrane. Suitably the IL-33 antagonist inhibits internalisation of EGFR. Suitably the IL-33 antagonist inhibits colocalisation of RAGE and EGFR within the cell membrane. Suitably the IL-33 antagonist inhibits internalisation of RAGE-EGFR complexes.

Suitably the IL-33 antagonist inhibits activation of EGFR. Suitably the IL-33 antagonist inhibits phosphorylation of EGFR.

Suitably the IL-33 antagonist inhibits RAGE-EGFR mediated effects. Suitably the IL-33 antagonist inhibits effects mediated by the RAGE-EGFR complex. Suitably the IL-33 antagonist inhibits effects mediated by the oxidised IL33-RAGE-EGFR complex.

Suitably the IL-33 antagonist inhibits EGFR signalling. Suitably the IL-33 antagonist inhibits RAGE-EGFR signalling. Suitably the IL-33 antagonist inhibits oxidised-IL33-RAGE-EGFR signalling.

Suitably the IL-33 antagonist inhibits binding of oxidised IL-33 to RAGE, thereby inhibiting RAGE-EGFR complexing, thereby inhibiting RAGE-EGFR mediated effects such as downstream signalling.

Suitably the IL-33 antagonist inhibits an IL-33-mediated EGFR effect. Suitably the IL-33 antagonist inhibits IL-33-mediated EGFR signalling. Suitably the IL-33 antagonist inhibits an oxidised IL-33-mediated EGFR effect. Suitably the IL-33 antagonist inhibits oxidised IL-33-mediated EGFR signalling. Suitably the IL-33 antagonist inhibits an oxidised IL-33-mediated RAGE-EGFR effect. Suitably the IL-33 antagonist inhibits oxidised IL-33-mediated RAGE-EGFR signalling.

Suitably a RAGE-EGFR mediated effect is caused by the RAGE-EGFR complex, suitably by the oxidised IL-33-RAGE-EGFR complex.

Suitably such effects may typically include downstream signalling which may be referred to herein as EGFR signalling or RAGE-EGFR signalling. Suitably such EGFR signalling may include phosphorylation and/or chemokine release.

Suitably such EGFR signalling includes phosphorylation of EGFR and subsequent phosphorylation of components in the EGFR pathway such as EGFR, PLC, JNK, MAPK/ERK 1/2, p38, and STAT5.

Suitably EGFR signalling includes phosphorylation of tyrosine kinases such as JNK, MAPK/ERK, p38.

Suitably EGFR signalling includes increased release of chemokines such as IL-8.

Therefore, suitably, the IL-33 antagonist inhibits EGFR mediated phosphorylation and/or chemokine release.

Therefore, suitably, the IL-33 antagonist inhibits phosphorylation of components in the EGFR pathway. Suitably, the IL-33 antagonist inhibits phosphorylation of any one of: EGFR, PLC, JNK, MAPK/ERK 1/2, p38, and STAT5. Suitably, the IL-33 antagonist inhibits EGFR-mediated phosphorylation of any one of: EGFR, PLC, JNK, MAPK/ERK 1/2, p38, and STAT5. Suitably, the IL-33 antagonist inhibits phosphorylation of tyrosine kinases. Suitably, the IL-33 antagonist inhibits phosphorylation of tyrosine kinases selected from: JNK, MAPK/ERK, p38. Suitably, the IL-33 antagonist inhibits EGFR-mediated phosphorylation of tyrosine kinases selected from: JNK, MAPK/ERK, and p38.

Therefore, suitably, the IL-33 antagonist inhibits release of chemokines. Suitably, the IL-33 antagonist inhibits release of IL-8. Suitably, the IL-33 antagonist inhibits EGFR-mediated release of chemokines. Suitably, the IL-33 antagonist inhibits EGFR-mediated release of IL-8.

In one embodiment, there is provided an IL-33 antagonist for use in the prevention or treatment of an EGFR-mediated disease.

In another embodiment, the IL-33 antagonist may be for use in the prevention or treatment of a respiratory disease by inhibiting an EGFR-mediated effect.

Furthermore, the IL-33 antagonist may be for use in the prevention or treatment of abnormal epithelium physiology, in an EGFR-mediated disease.

In one embodiment, there is provided an IL-33 antagonist for use in the prevention or treatment of an EGFR-mediated disease by improving abnormal epithelium physiology.

Suitably the EGFR-mediated disease is a RAGE-EGFR mediated disease.

Suitably the EGFR-mediated effect is a RAGE-EGFR mediated effect.

Suitably the EGFR-mediated effect is a RAGE-EGFR mediated signalling.

Suitably, the IL-33 antagonist inhibits an EGFR mediated effect. Suitably the IL-33 antagonist treats or prevents a disease or condition by inhibiting an EGFR-mediated effect.

Suitably, the IL-33 antagonist inhibits a RAGE-EGFR mediated effect. Suitably the IL-33 antagonist treats or prevents a disease or condition by inhibiting a RAGE-EGFR-mediated effect.

“RAGE-EGFR mediated effect” as recited herein refers to any physiological effect caused by the complexing of RAGE with EGFR in cell membranes and resulting aberrant EGFR activity. RAGE-EGFR mediated effects may also include and/or be referred to herein as ‘RAGE-EGFR signalling’ optionally ‘RAGE-EGFR mediated signalling’. Such RAGE-EGFR mediated effects are typically seen in the epithelium, and present as abnormal epithelium physiology. Abnormal epithelium physiology is defined hereinabove, but may include negative effects on: barrier integrity; regulation and exchange of chemical entities between tissues and a cavity; secretion of chemicals into a cavity; and sensation.

Suitably a RAGE-EGFR mediated disease and/or effects are characterised by aberrant EGFR activity. Suitably a RAGE-EGFR mediated disease and/or effects are characterised by aberrant RAGE-EGFR signalling. Suitably RAGE-EGFR mediated effects and/or RAGE-EGFR signalling are characteristics of a RAGE-EGFR mediated disease.

Suitably a RAGE-EGFR mediated disease may be a disease characterised by abnormal epithelium physiology.

Suitably a RAGE-EGFR mediated disease may be a disease characterised by abnormal epithelium physiology in respiratory epithelium.

Suitably a RAGE-EGFR mediated disease may be a disease characterised by abnormal mucociliary physiology.

Suitably a RAGE-EGFR mediated disease may be a disease characterised by abnormal mucociliary physiology in respiratory epithelium.

Suitable RAGE-EGFR mediated diseases may be selected from any of the respiratory diseases defined hereinbelow.

### Respiratory Diseases

The present disclosure relates to medical uses of IL-33 antagonist for the prevention or treatment of a respiratory disease by improving epithelium physiology or by modulating an EGFR-mediated effect, suitably, by inhibiting an EGFR-mediated effect, suitably, by inhibiting a RAGE/EGFR-mediated effect.

Suitably, the abnormal epithelium physiology may be a symptom of a respiratory disease. Suitably therefore, the IL-33 antagonist may be for use in the treatment or prevention of a respiratory disease characterised by abnormal epithelium physiology.

As defined in a further embodiment, there is provided an IL-33 antagonist for use in the prevention or treatment of a respiratory disease by improving epithelium physiology.

Abnormal epithelium physiology is defined elsewhere herein.

Suitably improving epithelium physiology may comprise improving abnormal epithelium physiology.

Suitable means of improving abnormal epithelium physiology are described hereinabove.

Suitably treatment of a respiratory disease by improving abnormal epithelium physiology may comprise:
improving or increasing mucociliary clearance;
reducing or inhibiting mucus production;
inhibiting abnormal mucus composition;
reducing or inhibiting abnormal epithelium remodelling; and/or
reducing or inhibiting goblet cell differentiation or proliferation.

Further details on each of these effects is provided hereinabove in relation to treatment or prevention of abnormal epithelium physiology, and may be combined here with the treatment of a respiratory disease.

Suitably, aberrant EGFR activity may be a symptom of a respiratory disease. Suitably therefore, the IL-33 antagonist may be for use in the treatment or prevention of a respiratory disease characterised by aberrant EGFR activity.

As defined in a further embodiment, there is provided an IL-33 antagonist for use in the prevention or treatment of a respiratory disease by inhibiting an EGFR-mediated effect.

EGFR-mediated effects are defined elsewhere herein.

Suitably the respiratory disease is a lower respiratory disease, suitably the respiratory disease is a disease which affects the trachea, bronchi, bronchioles, alveolar ducts and/or alveoli. Suitably the respiratory disease is a bronchial disease.

Suitably the respiratory disease may be selected from: COPD, bronchitis, emphysema, bronchiectasis, such as CF-bronchiectasis or non-CF-bronchiectasis, asthma, asthma and COPD overlap (ACO).

Suitably the respiratory disease is COPD. Suitably the respiratory disease is bronchitic COPD. Bronchitic COPD is a specific form of COPD in which chronic bronchitis is present in a patient with COPD. Bronchitic COPD causes greater mortality in patients than those with COPD due to faster lung function decline, increased symptoms, and increased risk of secondary infections. In particular, bronchitic COPD patients have higher total mucin concentrations and mucous hypersecretion. Therefore, bronchitic COPD patients may particularly benefit from treatment with an IL-33 antagonist as described herein by the discovery that IL-33 antagonists inhibit EGFR activity and improve mucociliary physiology.

Suitably, for the same reasons, the respiratory disease may be bronchitic asthma.

In one embodiment, there is provided an IL-33 antagonist for the prevention or treatment of bronchitic COPD.

In one embodiment, there is provided a method of prevention or treatment of bronchitic COPD in a patient, comprising: administering an effective amount of an IL-33 antagonist to a patient in need thereof.

### ST2 Signalling

Whilst the present disclosure relates to the medical use of an IL-33 antagonist to inhibit RAGE-EGFR mediated signalling and effects, it is already known that IL-33 antagonists can inhibit ST2-mediated signalling and effects. Therefore, the medical uses described herein envisage the modulation of both EGFR-mediated effects and ST2-mediated effects.

Suitably the IL-33 antagonist is for use in the prevention and treatment of abnormal epithelium physiology by modulating EGFR-mediated effects and ST2-mediated effects. Suitably the IL-33 antagonist is for use in the prevention and treatment of abnormal epithelium physiology by inhibiting EGFR-mediated effects and ST2-mediated effects. Suitably the IL-33 antagonist is for use in the prevention and treatment of abnormal epithelium physiology by inhibiting RAGE/EGFR-mediated effects and ST2-mediated effects. Abnormal epithelium physiology is as defined elsewhere herein.

Suitably the IL-33 antagonist is for use in the prevention and treatment of an EGFR-mediated disease by modulating EGFR-mediated effects and ST2-mediated effects. Suitably the IL-33 antagonist is for use in the prevention and treatment of an EGFR-mediated disease by inhibiting EGFR-mediated effects and ST2-mediated effects. Suitably the IL-33 antagonist is for use in the prevention and treatment of an EGFR-mediated disease by inhibiting RAGE/EGFR-mediated effects and ST2-mediated effects. EGFR-mediated diseases are as defined elsewhere herein.

Suitably, ST2-mediated effects include inflammation. Suitably, therefore the IL-33 antagonist is for use in the prevention and treatment of a ST2-mediated disease by modulating EGFR-mediated effects and ST2-mediated effects. Suitably the IL-33 antagonist is for use in the prevention and treatment of a ST2-mediated disease by inhibiting EGFR-mediated effects and ST2-mediated effects. Suitably the IL-33 antagonist is for use in the prevention and treatment of a ST2-mediated disease by inhibiting RAGE/EGFR-mediated effects and ST2-mediated effects. Suitable ST2 mediated diseases may include diseases characterised by inflammation. Suitable ST2 mediated diseases may include inflammatory diseases.

ST2-mediated diseases or inflammatory diseases may include: COPD; allergic disorders such as asthma, chronic rhinosinusitis, food allergies, eczema and dermatitis; fibroproliferative diseases such as pulmonary fibrosis; pulmonary eosinophilia; pleural malignancy; rheumatoid arthritis; collagen vascular disease; atherosclerotic vascular disease; uticaria; inflammatory bowel disease; Crohn's diseases; coeliac disease; systemic lupus; progressive systemic sclerosis; Wegner's granulomatosis; septic shock; and Bechet's disease.

Suitably the ST2-mediated disease or inflammatory disease is respiratory. Suitably the ST2-mediated disease or inflammatory disease is present in the respiratory tract as defined above.

Suitably the IL-33 antagonist is additionally for use in the prevention and treatment of inflammation or an inflammatory disease. Suitably, the IL-33 antagonist may be additionally for use in the prevention and treatment of ST2-mediated inflammation or an ST2-mediated inflammatory disease.

Suitably, the ST2-mediated disease and the EGFR-mediated disease overlap. In other words, ST2-mediated effects and EGFR-mediated effects, suitably RAGE-EGFR-mediated effects, contribute to disease pathology. Advantageously, it is believed that the medical use of a single IL-33 antagonist can inhibit both RAGE and ST2 activation by IL-33. Therefore, a single IL-33 antagonist can treat both a RAGE-EGFR mediated disease and an ST2 mediated disease at the same time.

In one embodiment there is provided an IL-33 antagonist for use in the prevention or treatment of abnormal epithelium physiology and inflammation. In one embodiment there is provided a method of prevention or treatment of abnormal epithelium physiology and inflammation in a patient comprising: administering an effective amount of an IL-33 antagonist.

Suitably the abnormal epithelium physiology and the inflammation may be symptoms of a respiratory disease. Accordingly statements about treatment and prevention of these symptoms may be in the context of a respiratory disease, and may suitably comprise the treatment or prevention of abnormal epithelium physiology and inflammation in a respiratory disease.

In one embodiment there is provided an IL-33 antagonist for use in the prevention or treatment of an EGFR-mediated disease and an ST2-mediated disease.

In one embodiment there is provided a method of prevention or treatment of an EGFR-mediated disease and a ST2-mediated disease in a patient comprising: administering an effective amount of an IL-33 antagonist.

Suitably the IL-33 antagonist is a reduced IL-33 antagonist. Suitably the reduced IL-33 antagonist is as defined hereinabove.

Suitably the IL-33 antagonist is as defined hereinabove. Suitably the IL-33 antagonist is 33_640087-7B.

Alternatively, different IL-33 antagonists could be used as a combination therapy to inhibit both RAGE and ST2 activation by IL-33. Therefore, a combination of IL-33 antagonists is envisaged to treat both a RAGE-EGFR mediated disease and an ST2 mediated disease at the same time.

Suitably the respiratory disease is as defined hereinabove. Suitably the respiratory disease is characterised by aberrant EGFR activity and aberrant ST2 activity.

Suitably, therefore, in one embodiment there is provided a first IL-33 antagonist for use in the prevention or treatment of abnormal epithelium physiology in combination with a second IL-33 antagonist for use in the prevention or treatment of inflammation.

Suitably, therefore, in one embodiment there is provided a method of prevention or treatment of abnormal epithelium physiology and inflammation in a patient, comprising: administering an effective amount of a first IL-33 antagonist in combination with an effective amount of a second IL-33 antagonist.

Suitably, therefore, in one embodiment there is provided a first IL-33 antagonist for use in the prevention or treatment of an EGFR mediated disease in combination with a second IL-33 antagonist for use in the prevention or treatment of an ST2-mediated disease.

Suitably, therefore, in one embodiment there is provided a method of prevention or treatment of an EGFR-mediated disease and an ST2-mediated disease in a patient, comprising: administering an effective amount of a first IL-33 antagonist in combination with an effective amount of a second IL-33 antagonist.

Suitably, the first IL-33 antagonist is for the prevention or treatment of abnormal epithelium physiology and/or an EGFR-mediated disease.

Suitably, the second IL-33 antagonist is for the prevention or treatment of inflammation and/or an ST2-mediated disease.

Suitably, the first and second IL-33 antagonists are different.

Suitably, the first IL-33 antagonist is as defined hereinabove. Suitably the second IL-33 antagonist may be any other IL-33 antagonist that is known to inhibit ST2 mediated effects. Suitably the second IL-33 antagonist is also defined herein above.

Suitably the first antagonist may be a reduced or oxidised IL-33 antagonist. Suitably the second IL-33 antagonist is a reduced IL-33 antagonist.

Suitably at least one of the IL-33 antagonists is 33_640087-7B. Suitably the first antagonist is 33_640087-7B.

Suitably the first and second IL-33 antagonists may be administered in combination. Suitably the first and second IL-33 antagonists may be administered in combination at the same time, or at different times. Suitable dosage regimes may be determined by the medical professional.

These statements apply equally to medical use/method of treatments mentioned above in which ST-2 mediated diseases may also be prevented or treated.

Alternatively, in further embodiments the IL-33 antagonist may be administered in combination with an ST2 inhibitor. Suitably the ST2 inhibitor may not be an IL-33 antagonist, but may inhibit the ST2 receptor by other means. Suitably the ST2 inhibitor may act to treat or prevent ST2-mediated diseases as identified above.

Therefore, in one embodiment there is provided an IL-33 antagonist for use in treatment or prevention of abnormal epithelium physiology in combination with an ST2 inhibitor for use in the treatment or prevention of inflammation.

In one embodiment, there is provided a method of prevention or treatment of abnormal epithelium physiology and inflammation in a patient, comprising: administering an effective amount of an IL-33 antagonist in combination with an effective amount of a ST2 inhibitor.

Suitably the abnormal epithelium physiology and the inflammation may be symptoms of a respiratory disease. Accordingly, statements about treatment and prevention of these symptoms may be in the context of a respiratory disease, and may suitably comprise the treatment or prevention of abnormal epithelium physiology and inflammation in a respiratory disease.

Therefore, in one embodiment there is provided an IL-33 antagonist for use in treatment or prevention an EGFR-mediated disease in combination with an ST2 inhibitor for use in the treatment or prevention of a ST2-mediated disease.

In one embodiment, there is provided a method of prevention or treatment of an EGFR-mediated disease in combination with an ST2-mediated disease in a patient, comprising: administering an effective amount of an IL-33 antagonist in combination with an effective amount of a ST2 inhibitor.

Suitably the IL-33 antagonist is as defined herein elsewhere. Suitable EGFR-mediated diseases and ST2-mediated diseases are defined elsewhere herein.

Suitably the ST2 inhibitor may be any such inhibitor known in the art, for example GSK3772847 (described in WO2013/165894) and RG6149 (WO2013/173761), both incorporated herein by reference.

Suitably the IL-33 antagonist and the ST2 inhibitor may be administered in combination. Suitably the IL-33 antagonist and the ST2 inhibitor may be administered in combination at the same time, or at different times. Suitable dosage regimes may be determined by the medical professional.

### Patient

The methods and medical uses are practiced in respect of a patient or subject. The patient may be one requiring identification, diagnosis or treatment for a physiological condition or disease such as abnormal epithelium physiology, an EGFR mediated disease, or a respiratory disease.

Suitably the patient may be a human. The patient may be undergoing medical care, or an individual requesting medical care. Suitably the patient is male or female. Suitably the patient is an adult or a child.

Suitably in the methods described herein, a suitable patient may be one believed to have abnormal epithelium physiology, or an EGFR mediated disease, or a respiratory disease. For example, a suitable patient may have symptoms consistent with such conditions.

Alternatively, a suitable patient in the context of the methods described herein may be one believed to be at risk of developing abnormal epithelium physiology, or an EGFR mediated disease, or a respiratory disease. For example, such a patient may have been in contact with an individual suffering from such a condition, may suffer from a related condition, or may satisfy risk factors associated with said conditions like smoking, old age, allergy etc.

### EMBODIMENTS

Parts of the disclosure may be characterized by the following embodiments, in which:

Embodiment 1 describes an IL-33 antagonist for use in the prevention or treatment of a disease by inhibiting an EGFR-mediated effect.

Embodiment 2 describes an IL-33 antagonist for use according to embodiment 1, wherein the EGFR-mediated effect is a RAGE-EGFR-mediated effect.

Embodiment 3 describes an IL-33 antagonist for use according to embodiment 1 or 2, wherein the EGFR-mediated effect is RAGE-EGFR-mediated signalling.

Embodiment 4 describes an IL-33 antagonist for use according to any of embodiments 1-3, wherein the disease is a respiratory disease.

Embodiment 5 describes an IL-33 antagonist for use according to any of embodiments 1-4, wherein the disease is characterised by abnormal epithelium physiology and/or aberrant EGFR activity.

Embodiment 6 describes an IL-33 antagonist for use according to any of embodiments 1-5, wherein the disease is selected from: COPD, bronchitis, emphysema, bronchiectasis, such as CF-bronchiectasis or -CF-bronchiectasis, asthma or asthma and COPD overlap (ACO).

Embodiment 7 describes an IL-33 antagonist for use according to any of embodiments 4-6, wherein the respiratory disease is bronchitic COPD.

Embodiment 8 describes an An IL-33 antagonist for use according to any of embodiments 1-7, wherein the treatment: improves mucus clearance; inhibits abnormal mucus production; inhibits abnormal epithelium remodelling; and/or inhibits abnormal goblet cell differentiation.

Embodiment 9 describes an IL-33 antagonist for use according to any preceding embodiment, wherein the IL-33 antagonist inhibits the activity of oxidised IL-33.

Embodiment 10 describes an IL-33 antagonist for use according to any preceding embodiment, wherein the IL-33 antagonist prevents binding of oxidised IL-33 to RAGE, thereby inhibiting RAGE-EGFR signalling.

Embodiment 11 describes an IL-33 antagonist for use according to any preceding embodiment, wherein the IL-33 antagonist is an anti-IL-33 antibody or antigen-binding fragment thereof, preferably an anti-reduced-IL-33 antibody or antigen-binding fragment thereof.

Embodiment 12 describes an IL-33 antagonist for use according to embodiment 11, wherein the anti-IL-33 antibody or antigen-binding fragment thereof comprises the complementarity determining regions (CDRs) of a variable heavy domain (VH) and a variable light domain (VL) pair selected from Table 1.

Embodiment 13 describes an IL-33 antagonist for use according to embodiment 12, wherein the anti-IL-33 antibody or antigen-binding fragment thereof comprises a variable heavy domain (VH) and variable light domain (VL) pair selected from Table 1.

Embodiment 14 describes an IL-33 antagonist for use according to any of embodiments 11-13, wherein the anti-IL-33 antibody or antigen-binding fragment thereof comprises a VHCDR1 having the sequence of SEQ ID NO: 37, a VHCDR2 having the sequence of SEQ ID NO: 38, a VHCDR3 having the sequence of SEQ ID NO: 39, a VLCDR1 having the sequence of SEQ ID NO: 40, a VLCDR2 having the sequence of SEQ ID NO: 41, and a VLCDR3 having the sequence of SEQ ID NO: 42.

Embodiment 15 describes an IL-33 antagonist for use according to any of embodiments 11-14, wherein the IL-33 antagonist is an anti-IL33 antibody or antigen binding fragment thereof comprising a VH domain of the sequence of SEQ ID NO:1 and a VL domain of the sequence of SEQ ID NO:19.

### BRIEF DESCRIPTION OF THE DRAWINGS

Embodiments will be described, by way of example, with reference to the following drawings, in which:

FIG. 1: shows a grayscale heat map of the fold increase in kinases phosphorylation, compared to untreated control, for each of the detection assays on the MAP kinase phosphorylation antibody array. Reduced IL-33 (IL-33-01 and IL-33-16, respectively) did not cause any signals above baseline. oxIL-33 (oxidised IL-33-01) caused increased phosphorylation in multiple kinases;

FIG. 2: shows the signal pattern for each stimulation condition on a receptor tyrosine kinase (RTK) activity array. oxIL-33 but not reduced IL33-01 and IL33-16, respectively) triggered a positive signal on the RTK array corresponding to epidermal growth factor receptor (EGFR). Dot intensity correlates with receptor tyrosine kinase phosphorylation;

FIG. 3A: shows pEGFR (Tyr1068) activity in normal human bronchial epithelial (NHBE) cells stimulated with increasing concentrations of IL-33 or EGFR ligands. oxIL-33, but not reduced IL-33 (IL33-01), promoted phosphorylation of the EGFR similarly to EGF, HB-EGF and TGFα;

FIG. 3B: shows pEGFR (Tyr1068) activity in A549 cells stimulated with increasing concentrations of IL-33 or EGFR ligands. oxIL-33 (oxidised IL-33-01), but not reduced IL-33 (IL-33-01) promoted phosphorylation of the EGFR similarly to EGF, HB-EGF and TGFα in a similar pattern to that seen in NHBE cells.

FIG. 3C: shows pEGFR (Tyr1068) activity in A549 cells stimulated with increasing concentrations of IL-33, EGFR ligands or RAGE ligands. oxIL-33, but not wild type (WT) IL-33 (IL-33-01), C->S mutated (mut) IL-33 (IL-33-16) or RAGE ligands, promoted phosphorylation of the EGFR similarly to EGF;

FIG. 4: shows that oxidised IL-33 induces the phosphorylation of multiple molecules involved in EGFR pathway (EGFR, PLC, AKT, JNK, ERK 1/2, p38) as analyzed by Western blot;

FIG. 5: shows STAT5 phosphorylation induced by oxIL-33-01 is reduced by increasing doses of anti-EGFR antibody as compared with isotype control;

FIG. 6: shows immunoprecipitation with anti-EGFR followed by detection of EGFR, RAGE or IL-33 by Western blot. IL-33 and RAGE co-precipitate with EGFR following NHBE stimulation with oxIL-33 suggesting that they form a complex. RAGE appears to be unique to the oxIL-33 signalling complex in comparison with EGF;

FIG. 7A: shows that oxIL-33 directly binds to RAGE. HMGB1 is a known RAGE ligand and acts as a positive control in this study;

FIG. 7B: show that oxIL-33 does not directly bind to EGFR (but the known EGFR ligand EGF does). However, when RAGE is added in to this assay in combination with oxIL-33 then EGFR binding is seen;

FIG. 8: shows immunoprecipitation with anti-EGFR or anti-RAGE, followed by western blot for EGFR, RAGE and IL-33 in wild type and RAGE-deficient A549 cells after activation with oxIL-33 at indicated time points;

FIG. 9: shows STAT5 phosphorylation induced by oxIL-33-01 is reduced by anti-RAGE antibody but not anti-ST2 antibody;

FIG. 10: shows EGF and oxidised IL33 (oxIL33) induce EGFR clustering and internalisation in EGFR-GFP A549 cells. Representative Images are shown after 5 minutes of stimulation. Histograms show depletion of EGFR in non-clustered area (left shift of the histogram bell shape peaks) in cells treated with EGF and oxIL-33, and increased numbers of saturated pixels (intensity 255) in these cells, caused by clustering.

FIG. 11: shows fold increase in IL-8 secretion by NHBES and DHBEs after 24 h stimulation with media alone (unstimulated control), 30 ng/ml IL-33-01, 30 ng/ml IL-33-16, 30 ng/mL oxidised IL-33 or 30 ng/mL EGF. Bar diagrams shows means and SEM from 4 NHBEs and 3 DHBEs donors;

FIG. 12A: shows relative wound healing density for A549 cells after treatment with reduced IL-33, oxIL-33 or EGF. Bar diagram shows mean and SEM from 6 technical replicates per condition;

FIG. 12B: shows relative wound healing density for NHBE cells after treatment with reduced IL-33, oxIL-33 or EGF. Bar diagram shows mean and SEM from 6 technical replicates per condition;

FIG. 13: shows percentage scratch wound closure of NHBE cells treated with media alone (unstimulated control), reduced IL-33, oxidised IL-33, or oxidised IL-33 in the presence of anti-ST2, anti-RAGE or anti-EGFR. Bar diagram shows mean and SEM from 6 technical replicates per condition;

FIG. 14: Shows relative wound healing density in human bronchial epithelial cells from healthy subjects, smokers and COPD, with and without stimulation with oxidised IL-33;

FIG. 15: Shows wound closure at 24 hours (%) of NHBE cells (n=5 donors) compared with DHBE COPD cells (n=5 donors) and DHBE treated with IgG1 control, Anti-IL-33 (33_640087-7B), anti-RAGE (M4F4) and anti-ST2. Bar diagram shows mean and SEM from n=5 individual donors;

FIG. 16A: shows a representative immunohistochemistry staining of basal (p63+; blue), ciliated (alpha tubulin; purple) and goblet (Mucin5ac+MucinB; yellow) from ALI cultures derived from healthy donors.

FIG. 16B: shows Quantification of immunohistochemistry of the various epithelial cell types using HALO software after 7 days of treatment with anti-IL-33 (33_640087-7B) or isotype control antibody; Data shown are mean and SEM from n=2-3 individual donors.

FIG. 16C: shows Quantification of goblet cells using HALO software after 7 days of treatment with anti-IL-33 (33_640087-7B) or isotype control antibody; Data shown are mean and SEM from n=2-3 individual donors.

FIG. 17: shows example staining for individual mucins (mucin5AC and mucin5B) in ALI cultures derived from heathy (1 donor) or COPD (1 donor) and reduction of mucin staining in COPD cultures after 7 days of treatment with anti-IL-33 (33_640087-7B).

FIG. 18: shows tSNE plots illustrating the different proportions of cell subtypes that are found in COPD ALI cultures from an individual donor treated with anti-IL-33 as compared to no treatment.

FIG. 19A: shows a representative flow-cytometry contour plot detecting goblet cells in ALI cultures from a normal donor. Muc5B is on the x-axis and Muc5AC is on the y-axis. ALI cultures were treated with proteins for 7 days. Treatment with reduced IL-33 (IL-33 [C->S]) did not increase goblet cells above baseline. oxIL-33 (oxidised IL-33-01) caused increased goblet cell percentages as did IL-13. IL-13 is known to increase goblet cells in ALI cultures and is used as a positive control in this study. Numbers in quadrants show percentages of total population: Muc5AC single-positive goblet cells in top-left quadrant, Muc5B single-positive goblet cells in lower-right quadrant and Muc5AC and Muc5B double-positive goblet cells in the top-right quadrant.

FIG. 19B: shows combined flow-cytometry data from ALI cultures from normal donors (n=6) showing percentage of goblet cells (combined Muc5AC single-positive, Muc5B single-positive and Muc5AC and Muc5B double-positive goblet cells) over the total epithelial population. Reduced IL-33 (IL-33[C->S]) did not increase goblet cells above baseline. oxIL-33 (oxidised IL-33-01) caused increases in goblet cell percentages as did IL-13. Violin plots show all data points and median.

FIG. 19C: shows combined flow-cytometry data from ALI cultures from normal donors (n=6) showing Muc5AC single-positive goblet cells. Reduced IL-33 (IL-33 [C->S]) did not increase goblet cells above baseline. oxIL-33 (oxidised IL-33-01) caused an increase in goblet cell percentages as did IL-13. Violin plots show all data points and median.

FIG. 19D: shows combined RT-qPCR data from ALI cultures from normal donors (n=4) showing fold-change in MUC5AC mRNA. Reduced IL-33 (IL-33 [C->S]) did not increase MUC5AC mRNA. oxIL-33 (oxidised IL-33-01) caused an increase in MUC5AC mRNA as did IL-13. Violin plots show all data points and median.

FIG. 20A: shows a representative immunohistochemistry staining of basal (p63+; purple), ciliated (alpha tubulin; teal) and goblet (Muc5ac+Muc5B; yellow) from ALI cultures derived from healthy donors. Reduced IL-33 (IL-33 [C->S]) did not visually increase goblet cells. oxIL-33 (oxidised IL-33-01) caused a visible increase in goblet cells.

FIG. 20B: shows quantification of mucin5ac+mucin5b area (% total epithelial tissue area) from immunohistochemistry images (a minimum of n=3 donors per condition), using HALO software. Compared with untreated and reduced IL-33 treated controls, oxIL-33 and IL-13 increase the area of mucin staining.

FIG. 21A: shows a representative flow-cytometry contour plot detecting goblet cells in ALI cultures from a COPD donor. Muc5B is on the x-axis and Muc5AC is on the y-axis are depicted. ALI cultures were treated with antibodies for 7 days. Anti-IL-33 (33_640087-7B) treatment reduced total goblet cell numbers. Numbers in quadrants show percentages of total population: Muc5AC single-positive goblet cells in top-left quadrant, Muc5B single-positive goblet cells in lower-right quadrant and Muc5AC and Muc5B double-positive goblet cells in the top-right quadrant

FIG. 21B: shows combined flow-cytometry data from ALI cultures from COPD donors (n=6) showing total goblet cells (combined Muc5AC single-positive, Muc5B single-positive and Muc5AC and Muc5B double-positive goblet cells). ALI cultures were treated with antibodies for 7 days. Anti-IL-33 (33_640087-7B) treatment reduced total goblet cell numbers. Violin plots show all data points and median.

FIG. 21C: shows combined flow-cytometry data from ALI cultures from COPD donors (n=6) showing Muc5AC single-positive goblet cells. ALI cultures were treated with antibodies for 7 days. Anti-IL-33 (33_640087-7B) treatment reduced Muc5AC single-positive goblet cell numbers. Violin plots show all data points and median.

FIG. 21D: shows combined RT-qPCR data from ALI cultures from COPD donors (n=5) showing fold-change in MUC5AC mRNA. Anti-IL-33 (33_640087-7B) treatment reduced MUC5AC expression. Violin plots show all data points and median.

FIG. 21E: shows combined flow-cytometry data from ALI cultures from COPD donors (n=6) showing total viability across treatment conditions as judge by LD negative cell staining.

FIG. 22A: shows a representative immunohistochemistry staining of basal (p63+; purple), ciliated (alpha tubulin; teal) and goblet (Muc5ac+MucB; yellow) from ALI cultures derived from a COPD donor. Anti-IL-33 (33_640087-7B) treatment for 7 days caused a visible reduction in goblet cells.

FIG. 22B: shows quantification of Muc5ac+Muc5b area (% total epithelial tissue area) from immunohistochemistry images (n=4 donors), using HALO software. Compared with untreated and human IgG1 treated controls, Anti-IL-33 (33-640087_7B) reduces the area of mucin staining.

FIG. 23A: shows quantification of Muc5AC within apical washes obtained from COPD and Healthy ALI cultures. Muc5AC levels are higher in COPD cultures as judge by Muc5AC ELISA

FIG. 23B: shows quantification of Muc5AC within apical washes obtained from Healthy ALI cultures. ALI cultures were treated with reduced IL-33mut16 (IL-33 [C->S]), oxIL-33 and wildtype IL-33 as determined by Muc5AC ELISA.

FIG. 23C: shows quantification of Muc5AC within apical washes obtained from COPD ALI cultures. Cells were treated with human and mouse IgG1 controls (hIgG1 and mIgG1), 33-640087_7B or an anti-ST2 antibody. Treatment with Anti-IL-33 (33-640087_7B) reduced Muc5AC levels as determined by Muc5AC ELISA.

### EXAMPLES

### Example 1—Oxidised IL-33 Drives Formation of a Signaling Complex Between RAGE and EGFR

In Cohen, E. S. et al. Nat. Commun. 6:8327 (2015), the applicant described the discovery of an oxidized, disulphide bonded form of IL-33 (DSB IL-33) and showed that this form does not bind ST2 and cannot activate ST2-dependent signalling. Subsequently (see WO2016156440A1), the applicant showed that oxIL-33 binds the Receptor for Advanced Glycation End products (RAGE) and signals in a RAGE-dependent manner to activate STAT5 and affect epithelial migration.

To further explore the function of oxIL-33, epithelial cells were stimulated with IL-33 in reduced or oxidised forms and signaling pathways were investigated. Here the inventors show that oxIL-33 is a novel ligand for a complex of the receptor for advanced glycation end products (RAGE) and the epidermal growth factor receptor (EGFR), leading to profound effects on epithelial function.

1. Cloning and Expression of Human Mature and Cysteine-Mutated Variants of IL33

cDNA molecules encoding the mature component of human IL-33 (112-270); accession number (UniProt) 095760 (also referred to as IL33-01 or IL-33), and a variant with the 4 cysteine residues mutated to serine (also referred to as IL33-16 or IL-33[C->S]) were synthesized by primer extension PCR and cloned into pJexpress 411 (DNA 2.0). The wild type (WT) and mutant IL-33 coding sequences were modified to contain a 10×His, Avitag, and Factor-Xa protease cleavage site (MHHHHHHHHHHAAGLNDIFEAQKIEWHEAAIEGR SEQ ID NO:43) at the N-terminus of the proteins. N-terminal tagged His10/Avitag IL33-01 (WT, SEQ ID NO:44) and N-terminal tagged His10/Avitag IL33-16 (WT, SEQ ID NO:45) were generated by transforming E. coli BL21(DE3) cells. Transformed cells were cultured in autoinduction media (Overnight Express™ Autoinduction System 1, Merck Millipore, 71300-4) at 37° C. for 18 hours before cells were harvested by centrifugation and stored at −20° C. Cells were resuspended in 2×DPBS containing complete EDTA-free protease inhibitor cocktail tablets (Roche, 11697498001) and 50 U/ml Benzonase nuclease (Merck Millipore, 70746-3) and lysed by sonication. The cell lysate was clarified by centrifugation at 50,000×g for 30 min at 4° C. IL-33 proteins were purified from the supernatant by immobilized metal affinity chromatography, loading on a HisTrap excel column (GE Healthcare, 17371205) equilibrated in 2×DPBS, 1 mM DTT at 5 ml/min. The column was washed with 2×DPBS, 1 mM DTT, 20 mM Imidazole, pH 7.4 to remove impurities and then 2×DPBS, 0.1% Triton X-114 to deplete the immobilised protein of endotoxin. Following further washing with 2×DPBS, 1 mM DTT, 20 mM Imidazole, pH 7.4, the sample was eluted with 2×DPBS, 1 mM DTT, 400 mM Imidazole, pH 7.4. IL-33 was further purified by size exclusion chromatography using a HiLoad Superdex 75 26/600 pg column (GE Healthcare, 28989334) in 2×DPBS at 2.5 ml/min. Peak fractions were analysed by SDS PAGE. Fractions containing pure IL-33 were pooled and the concentration determined by absorbance at 280 nm. Final samples were analysed by SDS-PAGE.

To generate untagged IL-33, N-terminal tagged His10/Avitag IL33 was incubated with 10 units of Factor Xa (GE healthcare, 27084901) per mg of protein in 2×DPBS buffer at RT for 1 hour. Untagged IL-33 was purified using SEC chromatography in 2×DPBS on a HiLoad 16/600 Superdex 75 pg column (GE healthcare, 28989333) with a flow rate of 1 ml/min.

2. Generation and Purification of Oxidised IL-33 (oxIL-33)

Reduced IL33-01 was oxidised by dilution to a final concentration of 0.5 mg/ml in 60% IMDM medium (with no phenol red), 40% DPBS and incubation at 37° C. for 18 hours. Aggregates generated during the oxidation process were removed from the sample by loading it on a HiTrap Capto Q ImpRes anion exchange column (GE Healthcare, 17547055). Prior to loading, the sample was modified by the addition of 1 M Tris, pH 9.0 until the pH reached 8.3 and the addition of 5 M NaCl to a final concentration of 125 mM—under these loading conditions, aggregates bound to the column and monomeric oxIL-33 flowed through without binding and was collected. Tags were cleaved from the oxIL-33 by incubation with Factor Xa (NEB, P8010L) at a final concentration of 1 μg Factor Xa per 50 μg of oxIL-33 for 120 min at 22° C. To deplete the sample of any remaining reduced IL-33, soluble human ST2S extracellular domain fused to human IgG1 Fc-His6 was incubated with the sample for 30 min at 22° C. and bound the reduced IL-33. The sample was concentrated in a centrifugal concentrator with a 3,000 Da cut-off and loaded on a HiLoad Superdex 75 26/600 pg column (GE Healthcare, 28989334) at a flow rate of 2 ml/min, which separated the monomeric oxIL-33 from the other sample components. Fractions containing pure oxIL-33 were pooled and concentrated and the final concentration of the sample was determined via UV absorbance spectroscopy at 280 nm. Final product quality was assessed by SDS-PAGE, HP-SEC and RP-HPLC.

3. Cloning, Expression and Purification of Human ST2 ECD

A cDNA encoding the naturally occurring ST2S soluble isoform of ST2 (UniProt accession Q01638-2) without the endogenous signal peptide (amino acid residues 19-328) was amplified by PCR with primers encoding extensions compatible with Gibson assembly and a CD33 signal peptide fused to the N-terminus of the ST2S coding sequence. A coding sequence for human IgG1 Fc with a C-terminal His6-tag was similarly amplified. The ST2S cDNA and IgG1 Fc-His6 cDNA were assembled using Gibson assembly with pDEST12.2 OriP, a mammalian, CMV-promoter driven expression vector bearing the OriP origin of replication from EBV, allowing episomal maintenance in cell lines expressing the EBNA-1 protein. For protein expression, the plasmid was transiently transformed into a suspension culture of CHO cells overexpressing EBNA-1 using polyethyleneimine as the transfection reagent. Conditioned medium containing the secreted ST2S-Fc-His6 fusion protein was collected 7 days post-transfection and loaded on a HiTrap MabSelect SuRe (Protein A, GE Healthcare, 11-0034-95) affinity chromatography column at 2 ml/min. The column was washed with 2×DPBS and the protein eluted with 25 mM Sodium acetate, pH 3.6. Fractions containing ST2S-Fc-His6 were pooled and loaded on a HiLoad Superdex 200 26/600 pg column (GE Healthcare, 28989336) equilibrated in 2×DPBS at 2 ml/min. Fractions containing pure ST2S-Fc-His6 protein were pooled and the concentration determined by absorbance at 280 nm. Final samples were analysed by SDS-PAGE.

4. Cloning, Expression and Purification of Human Asialoglycoprotein Receptor (ASGPR) ECD

A cDNA encoding the extracellular domain (ECD) of the Asialoglycoprotein receptor (UniProt accession P07306) without the cytoplasmic and transmembrane domains (amino acid residues 62-291) was chemically synthesized at Geneart with a CD33 signal peptide followed by an His10_Avi Tag sequence fused to the N-terminus of the ECD domain. The construct was cloned directly into pDEST12.2 OriP, a mammalian, CMV-promoter driven expression vector bearing the OriP origin of replication from EBV, allowing episomal maintenance in cell lines expressing the EBNA-1 protein. For protein expression, the plasmid was transiently transformed into a suspension culture of HEK Freestyle 293F cells using 293 Fectin as the transfection reagent. Conditioned medium containing the secreted HisAVi_hASGPR ECD fusion protein was collected 7 days post-transfection by immobilized metal affinity chromatography, loading on a HisTrap excel column (GE Healthcare, 17371205) equilibrated in 2×DPBS, at 4 ml/min. The column was washed with 2×DPBS, 40 mM Imidazole, pH 7.4 to remove impurities and the sample was eluted with 2×DPBS, 400 mM Imidazole, pH 7.4. Human ASGPR ECD was further purified by size exclusion chromatography using a HiLoad Superdex 75 16/600 pg column (GE Healthcare, 28-9893-33) in 2×DPBS at 1 ml/min. Peak fractions were analysed by SDS PAGE. Fractions containing pure monomeric ASGPR were pooled and the concentration determined by absorbance at 280 nm. Final samples were analysed by SDS-PAGE.

5. The Oxidised Form of IL-33 Activates MAP Kinase Pathways

Normal Human Bronchial Epithelial (NHBE) cells (CC-2540) were obtained from Lonza and were maintained in complete BEGM media (Lonza) according to the manufacturer's protocol. NHBEs were harvested with accutase (PAA, #L11-007) and seeded at 1×10⁶/2 ml in a 6-well dish (Corning Costar, 3516) in culture media [BEGM (Lonza CC-3171) and supplement kit (Lonza CC-4175)]. Cells were incubated at 37° C., 5% CO₂ for 18-24 hours. After this time, media was aspirated, and the cells were washed twice with 1 ml PBS before the addition of starve media (BEGM (Lonza CC-3171) supplemented with 1% Penicillin/Streptomycin). The plates were then incubated at 37° C., 5% CO₂ for a further 18-24 hours before stimulation.

MAP kinase phosphorylation antibody array kits (ab211061) were purchased from Abcam and experiments were carried out as per the manufacturer's instructions. NHBEs in a 6 well dish that had been starved for 18-24 h were left untreated or treated with 30 ng/ml of either reduced IL-33, IL-33-16 or oxidised IL-33 before being returned to an incubator 37° C., 5% CO₂ for 10 mins (see Table 2 for activators used in this assay). The plates were removed from the incubator and the cells washed with ice-cold PBS before the addition of 100 μl/per well of 1× lysis buffer supplied with the kits. Protein extracts were transferred to 1.5 ml tubes before being clarified at 14,000 rpm at 4° C. Protein concentration was determined using the BCA technique (Thermo, 23225) and 250 μg of total protein was used per array membrane. All subsequent steps were carried out following the manufacturer's instructions. Membranes were visualised on a LiCor C-digit and quantified using Image Lite studio.

TABLE 2

| | | | Final conc |
|---|---|---|---|
| Agonist | Identifier | Reconstitute in | (μg/ml) |
| Untagged oxidised IL33-01 | RD15 | PBS | 100 |
| Untagged IL33-01 | Jul. 24, 2015 | PBS | 100 |
| Untagged IL33-16 | Nov. 12, 2015 | PBS | 100 |
| EGF | 236-EG-200 | PBS | 100 |

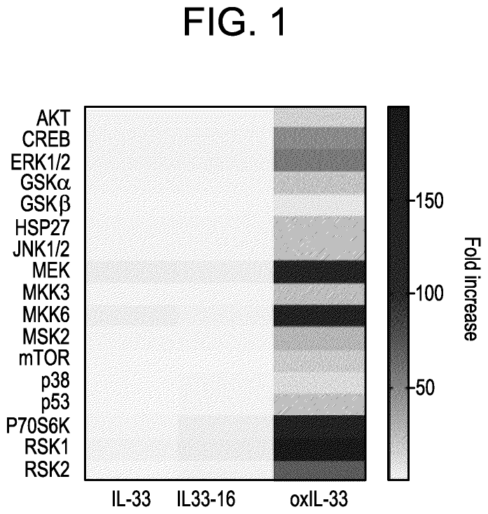

In contrast to the wild type (IL-33) and C->S (IL-33[C->S]) reduced forms of IL-33 (IL33-01 and IL33-16, respectively), oxidised IL33-01 (oxIL-33) activated multiple key signalling molecules (FIG. 1) coinciding with pathways engaged by receptor tyrosine kinases (RTK).

6. The Oxidised Form of IL-33 Activates Epidermal Growth Factor Receptor (EGFR)

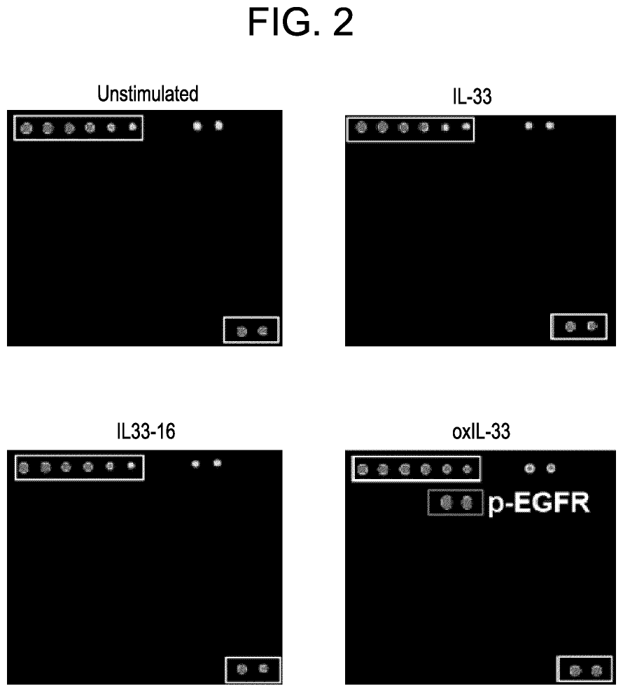

To try and identify receptor tyrosine kinases (RTK) that were activated by oxIL-33, screening was performed using a 71 RTK array. RTK phosphorylation antibody array kits (ab193662) were purchased from Abcam and experiments were carried out as per the manufacturer's instructions. NHBEs were cultured and seeded at 1×10⁶/2 ml in a 6-well plate (Corning Costar, 3516) in culture media [BEGM (Lonza CC-3171) and supplement kit (Lonza CC-4175)]. Cells were incubated at 37° C., 5% CO₂ for 18-24 hours. After this time, media was aspirated, and the cells were washed twice with 1 ml PBS before the addition of starve media (BEGM (Lonza CC-3171) without supplement kit). The plates were then incubated at 37° C., 5% CO₂ for a further 18-24 hours before stimulation. Following the same steps previously described for the MAP kinase array, cells were activated (Table 2 activators), lysed and 250 μg of total protein was used per array membrane. All subsequent steps were carried out following the manufacturer's instructions. Membranes were visualised on a LiCor C-digit and quantified using Image Lite studio. There was no response detected to either reduced wild type (IL-33) or C->S (IL-33[C->S]) IL-33 (IL33-01 and IL33-16, respectively). However, oxIL-33 (oxidised IL-33-01) triggered a positive signal on the RTK array corresponding to epidermal growth factor receptor (EGFR) (FIG. 2).

The ability of oxIL-33 (oxidised IL-33-01) to stimulate EGFR signalling was confirmed by additional methods. Upon activation, EGFR is phosphorylated at Tyr1068 and this phospho-EGFR can be detected using a homogeneous FRET (fluorescence resonance energy transfer) HTRF® (Homogeneous Time-Resolved Fluorescence, Cisbio International) assay (Cisbio kit #64EG1PEH). Briefly, NHBEs were plated at 5×10⁵/100 μl in a 96-well plate (Corning Costar, 3598) in culture media [BEGM (Lonza CC-3171) and supplement kit (Lonza CC-4175)]. The plates were incubated at 37° C., 5% CO₂ for 18-24 hours. After this time, media was aspirated, and the cells were washed twice with 0.2 ml PBS before the addition of starve media (BEGM (Lonza CC-3171) without supplement kit). The plates were then incubated at 37° C., 5% CO₂ for a further 18-24 hours before stimulating with increasing concentrations of IL-33-01, IL-33-16 and oxIL-33 (oxidised IL-33-01) and EGFR ligands (Tables 2 & 3) before being returned to an incubator 37° C., 5% CO₂ for 10 mins. The media was aspirated and replaced with 50 μl of lysis buffer per well (Cisbio, 64EG1PEH). The assay was then carried out as per the manufacturer's instructions (Cisbio, 64EG1PEH). Time resolved fluorescence was read at 620 nm and 665 nm emission wavelengths using an EnVision plate reader (Perkin Elmer). Data were analysed by calculating the 665/620 nm ratio and EC50 values determined using GraphPad Prism software by curve fitting using a four-parameter logistic equation.

TABLE 3

| | | | | Final conc |
|---|---|---|---|---|
| Agonist | Supplier | Identifier | Reconstituted in | (μg/ml) |
| TGFα | R&D | 239-A-100 | 10 mM | 100 |
| | systems | | acetic acid | |
| HB-EGF | R&D | 259-HE-050/CF | PBS | 100 |
| | systems | | | |
| Amphiregulin | R&D | 262-AR-100/CF | PBS | 100 |
| (AREG) | systems | | | |
| Betacellulin/ | R&D | 261-CE-010/CF | PBS | 100 |
| BTC | systems | | | |
| Epiregulin | R&D | 1195-EP-025/CF | PBS | 100 |
| | systems | | | |
| Epigen | R&D | 6629-EP-025/CF | PBS | 100 |
| | systems | | | |
| HMGB1 | R&D | 1690-HMB-050 | PBS | 200 |
| | systems | | | |
| S100A8/A9 | R&D | 8226-S8-050 | PBS | 500 |
| | systems | | | |
| S100A12 | R&D | 1052-ER-050 | PBS | 200 |
| | systems | | | |
| S100B | R&D | 1820-SB-050 | PBS | 200 |
| | systems | | | |

Similarly, EGFR phosphorylation was assessed in the epithelial cell line A549 utilizing HTRF assay as previously mentioned in this section. Briefly, A549s were obtained from ATCC and cultured in RPMI GlutaMax medium supplemented with 1% Penicillin/Streptomycin and 10% FBS. Cells were harvested with accutase (PAA, #L11-007) and seeded into 96 well plates at 5×10⁵/100 μl and incubated at 37° C., 5% CO₂ for 18-24 hours. The wells were then washed twice with 100 μl of PBS before addition of 100 μl of starve media (RPMI GlutaMax medium supplemented with 1% Penicillin/Streptomycin) and incubated at 37° C., 5% CO₂ for 18-24 hours. Cells were stimulated with increasing concentrations of IL-33-01, IL-33-16 and oxIL-33-01 (synonym of oxidised IL-33-01), EGFR ligands and RAGE ligands (Tables 2 & 3) before being returned to an incubator 37° C., 5% CO₂ for 10 mins. The media was aspirated and replaced with 50 μl of lysis buffer per well (Cisbio, 64EG1PEH). The assay was then carried out as per the manufacturer's instructions (Cisbio, 64EG1PEH). Time resolved fluorescence was read at 620 nm and 665 nm emission wavelengths using an EnVision plate reader (Perkin Elmer). Data were analysed by calculating the 665/620 nm ratio and EC50 values determined using GraphPad Prism software by curve fitting using a four-parameter logistic equation.

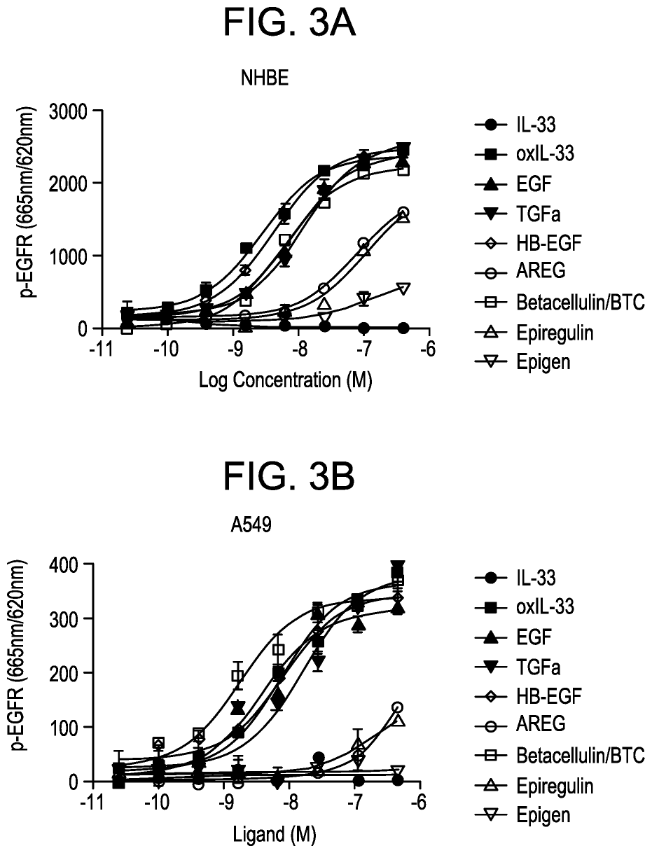

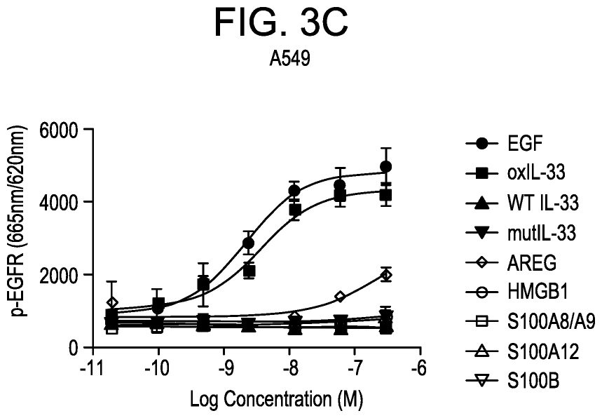

In both NHBE and A549 cells, oxIL-33 promoted phosphorylation of the EGFR similarly to a bona fide agonist, EGF (FIG. 3). This was not replicated by other RAGE ligands tested.

7. Western Blotting of Signaling Components

Western blot experiments were performed to further investigate which elements of the EGFR signalling complex are activated in response to oxIL-33 (oxidised IL33-01). NHBEs were cultured and plated in 6 well dishes as described above in section 5. Following serum starvation, cells were stimulated with oxIL-33 (30 ng/ml) for between 5 to 240 minutes. The media was then aspirated and the cells were washed with ice-cold PBS before the addition of 150 μl of lysis buffer [1×LDS sample buffer (Thermo, NP0008), 10 mM MgCl2 (VWR, 7786-30-3), 2.5% β-mercaptoethanol (Sigma, M6250) and 0.4 mg/ml benzonase (Millipore, 70746)]. Cells were left on ice for 10 mins before lysate was transferred to 1.5 ml tubes and heated to 90° C. for 5 mins. Solutions were transferred to new 1.5 ml tubes and 10 μl of sample along with 5 μl of protein ladder (BioRad, 1610374) was run on a 4-12% SDS-PAGE gel (Thermo, NW04127BOX) in IVIES running buffer (B0002). Gels were transferred onto PVDF membranes (BioRad, 1704156) using a Transblot Turbo (BioRad). PVDF membranes were blocked in PBS-tween solution containing 5% skimmed milk powder (Marvel) for 10 minutes. Membranes were then incubated with primary antibodies in PBS-tween containing 5% BSA over night at 4° C. The membranes were then washed five times with PBS-tween and then incubated with secondary HRP tagged antibodies in PBS-tween containing 5% skimmed milk powder for 1 hour at room temperature. The membranes were then washed five times with PBS-tween before the addition of ECL (BioRad, 1705062) and visualisation of a Licor C-digit.

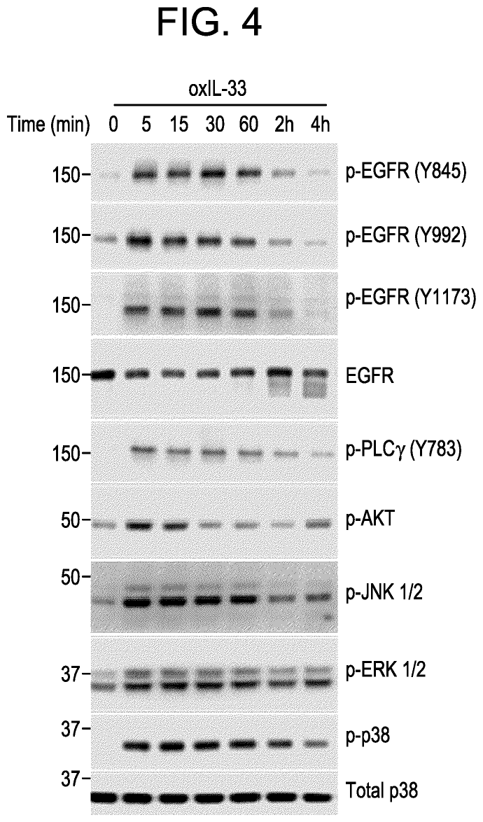

The results show that oxIL-33-01 activated several EGFR signaling components (FIG. 4)

8. Ox-IL-33 Induces STAT-5 Phosphorylation, which is Blocked by EGFR-Neutralizing Ab

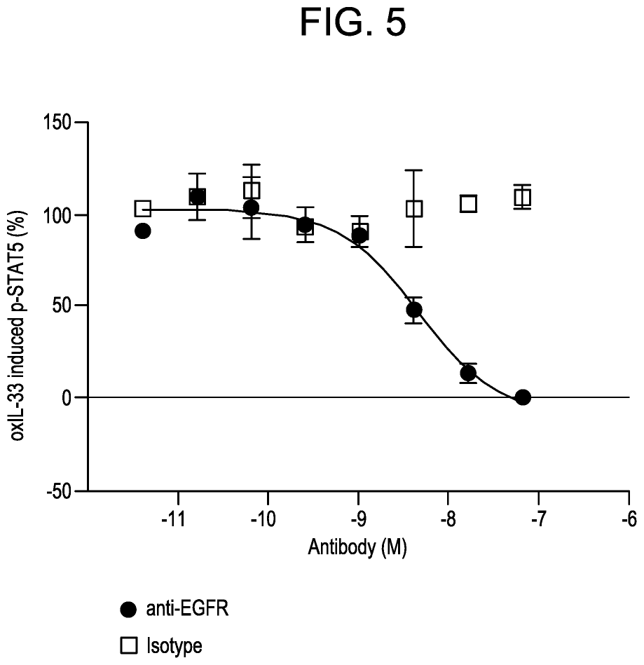

It was next sought to establish whether oxIL33-mediated STAT5 activation could be inhibited by preventing binding to EGFR. Briefly, A549 cells were cultured in RPMI GlutaMax medium supplemented with 1% Penicillin/Streptomycin and 10% FBS. Cells were harvested with accutase and seeded into 96 well plates at 5×10⁵/100 μl and incubated at 37° C., 5% CO₂ for 18-24 hours. The wells were then washed twice with 100 μl of PBS before addition of 100 μl of starve media (RPMI GlutaMax medium supplemented with 1% Penicillin/Streptomycin) and incubated at 37° C., 5% CO₂ for 18-24 hours. Anti-EGFR antibody (Clone LA1 (05-101, Millipore) or isotype control (MAB002, R&D Systems) was added in a dose dependent manner to the wells and the plate was returned to the incubator for 30 mins. The plates were then stimulated with oxidised IL-33 (30 ng/ml) for 30 mins before lysis using the phosho-STAT5 ELISA kit lysis buffer (85-86112-11, ThermoFischer Scientific) and developed following manufacturer's instructions before reading absorbance at 450 nM. As shown in FIG. 5, cells activated with oxIL-33-01 display phosphorylation of STAT5, which decreases in the presence of anti-EGFR antibody (FIG. 5).

### Example 2—Oxidised IL-33 Induces Complex Formation Between EGFR and RAGE

9. OxIL-33 induces complex formation between EGFR and RAGE In order to understand how RAGE and EGFR are involved in promoting signaling of oxIL-33, immunoprecipitation experiments were performed to explore the signaling complex. Firstly, anti-EGFR antibodies were covalently coupled to Dynabeads. Two 100 pg vials of anti-EGFR antibodies (R&D systems, AF231) were incubated with 40 mg of Dynabeads (Thermo, 14311D) and covalently coupled as per the manufacturer's instructions. Following successful coupling the beads were resuspended in PBS at 30 mg/ml and kept at 4° C.

NHBEs were obtained from Lonza (CC-2540) and frozen vials seeded directly into 15 cm dishes (Thermo, 157150) at 1×10⁶ cells per dish. NHBEs were maintained in complete BEGM media (Lonza) according to the manufacturer's protocol for one month with a media change every three days until the cells reached confluency. The plates were incubated at 37° C., 5% CO₂ for the duration of this time. The day before stimulation, the plates were washed twice with 20 ml PBS before the addition of 15 ml starve media (BEGM (Lonza CC-3171) without supplement kit). The plates were then incubated at 37° C., 5% CO₂ for a further 18-24 hours before stimulation with media alone (unstimulated control), 30 ng/ml reduced IL-33-01, 30 ng/mL oxidised IL-33-01 or 30 ng/mL EGF and returned to 37° C., 5% CO₂ for 10 mins. Media was aspirated, and the plates were washed twice with ice-cold PBS before the addition of 1 ml lysis buffer (Abcam, ab152163) containing phosphatase and protease inhibitors (Thermo, 78440) per 15 cm dish. The cells were scraped into the lysis buffer before being transferred into 2 ml Protein LoBind tubes (Eppendorf, Z666513) and clarified by spinning at 14,000 rpm at 4° C. Protein concentration was determined using a BCA kit (Thermo, 23225) and all protein extracts were normalised to 3 mg/ml with lysis buffer. 6 mg of total protein extract was incubated in a clean 2 ml LoBind tube with 100 μl of anti-EGFR Dynabeads (described above). The tubes were then placed on an end-over-end mixer at 4° C. for 5 h. Using a magnet (BioRad, 1614916) the Dynabeads were immobilised and the protein extract was aspirated and replaced with 2 ml wash buffer 1 (50 mM Tris-HCl pH 7.5 (Thermo, 15567027), 0.5% TritonX 100 (Sigma, X100), 0.3 M NaCl. This was repeated four more times. The beads were then washed a further ten times in the same manner with wash buffer 2 (50 mM Tris-HCl pH 7.5). After the final washing step, 50 μl of 1% Rapigest (w/v) (Waters, 186001861), in 50 mM Tris-HCl pH8.0, was added to the beads and heated at 60° C. for 10 min. The supernatant was then transferred to a new LoBind 2 ml tube. A further 100 μl of 50 mM Tris-HCl pH8.0 was added to the resin and mixed before it was combined with the first elution. TCEP (Sigma, 646547) was then added to a final concentration of 5 mM and the sample was heated at 60° C. for 10 min. The eluates were then alkylated by addition of iodoacetamide (Sigma, 16125) to 10 mM in the dark at room temperature for 20 min. The alkylation was quenched by the addition of DTT (Sigma, D5545) to 10 mM. Tris-HCl buffer 50 mM pH8.0 was then added to give a final sample volume of 500 μl. 0.5 μg of trypsin (Promega, V5111) per tube was added and samples were digested at 30° C. overnight at on a shaking platform at 400 rpm. The samples were then acidified with trifluoroacetic acid (Sigma, 302031) to a final concentration of 2.0% (v/v) and incubated at 37° C. for 1 h. Samples were then centrifuged at 14,000 rpm for 30 min and the supernatant was transferred to a new 2 ml LoBind tube. Samples were then processed through C18 columns (Thermo, 87784) as per the manufacturer's instructions. Samples were then dried using a speed-vac before being stored at −20° C. Samples were then analysed by peptide mass fingerprinting mass spectrometry (PMF-LC-MS). Scaffold software was used to analyse the results.

EGFR was detected similarly across all 4 conditions suggesting that the immunoprecipitation had worked well across all the samples. RAGE and IL-33 were detected in samples that had been treated with oxIL-33, in contrast to those treated with IL33-01 (IL-33) or EGF, suggesting that oxIL-33 and RAGE were associated with EGFR during signaling. Consistent with prior observations of EGFR activation in these cells with oxIL-33 and EGF, proteins previously reported to be involved in EGFR signaling and endocytosis were detected after stimulation with these ligands, but not reduced IL33-01 (Table 4).

Table 4 shows LCMS analysis of NHBE stimulated with reduced IL-33-01 (IL-33), oxIL-33 (oxidised IL-33-01) or EGF. IL-33 and RAGE are detected in complex with EGFR following stimulation with oxIL-33, but not after stimulation with reduced IL33-01 (IL-33) or EGF. Parentheses indicate the number of unique peptides identified for each protein.

TABLE 4

| Unstimulated | IL-33 | oxIL-33 | EGF |
|---|---|---|---|
| EGFR (63) | EGFR (62) | EGFR (60) | EGFR (57) |
| — | — | IL-33 (11) | — |
| — | — | RAGE (11) | — |
| — | — | AP-2α1 (20) | AP-2α1 (14) |
| — | — | AP-2α2 (16) | AP-2α2 (10) |
| — | — | AP-2β (15) | AP-2β (16) |
| — | — | AP-2μ (20) | AP-2μ (20) |
| — | — | AP-2σ (10) | AP-2σ (11) |
| — | — | CBL-B (5) | CBL-B (4) |

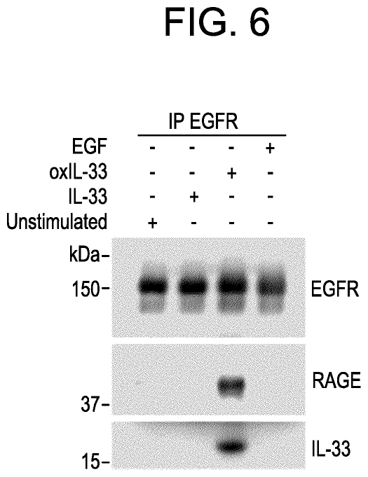

To confirm these observations, Immunoprecipitation and Western blotting was also performed on cell lysates prepared according to the above protocol. Following NHBE protein extract concentration determination, 3 mg of total protein was incubated in a 1.5 ml tube with 6 μg of anti-EGFR antibody (R&D systems, AF231) and placed on an end-over-end mixer at 4° C. for 2.5 h. 1.5 mg of protein A/G magnetic beads (Thermo, 88802) were then added to each tube and the tubes were then returned to 4° C. for another 1 h with mixing. The beads were then collected with a magnet (BioRad, 1614916) and washed three times with 500 μl of (50 mM Tris (pH 7.5), 1% TritonX and 0.25 M NaCl) and once with 500 μl of 10 mM Tris (pH 7.5). The proteins were then released from the magnetic beads using 35 μl of LDS sample buffer (Thermo, NP0008) with reducing agent (Thermo, NP0004) and heating at 95° C. for 5 minutes. Solutions were transferred to new 1.5 ml tubes and 10 μl of sample along with 5 μl of protein ladder (BioRad, 1610374) was run on a 4-12% SDS-PAGE gel (Thermo, NW04127BOX) in IVIES running buffer (B0002). Gels were transferred onto PVDF membranes (BioRad, 1704156) using a Transblot Turbo (BioRad). PVDF membranes were blocked in PBS-tween solution containing 5% skimmed milk powder (Marvel) for 10 minutes. Membranes were then incubated with primary antibodies (anti-EGFR (Cell Signaling Technology, 2232), anti-RAGE (Cell Signaling Technology, 6996) or anti-IL-33 (R&D systems, AF3625) in PBS-tween containing 5% BSA over night at 4° C. The membranes were then washed five times with PBS-tween and then incubated with anti-rabbit secondary HRP tagged antibodies (Cell Signalling Technology, 7074) or anti-goat secondary HRP tagged antibodies (R&D systems, HAF109) in PBS-tween containing 5% skimmed milk powder for 1 hour at room temperature. The membranes were then washed five times with PBS-tween before the addition of ECL (BioRad, 1705062) and visualisation of a Licor C-digit. Western blotting confirmed that RAGE coprecipitated with EGFR in the presence of oxIL-33 whereas no RAGE was detected with EGF stimulation (FIG. 6). These findings reveal that RAGE and EGFR are a functional part of the oxidized IL-33 signaling complex.

10. RAGE is Required for oxIL-33 to Form a Complex with EGFR

The experiments described above have shown that oxIL-33 is a ligand for a complex of the EGF Receptor (EGFR), which results in downstream signaling. The experiments in this section are designed to determine whether oxIL-33 is a direct binding ligand for either RAGE or EGFR. To understand more about the formation of the signaling complex and assess whether oxIL-33 directly interact with EGFR, an ELISA format was used to explore binding of oxIL-33 to RAGE, ST2-Fc and EGFR.

Proteins and Modifications: Proteins containing the Avitag sequence motif (GLNDIFEAQKIEWHE SEQ ID NO:46) were biotinylated using the biotin ligase (BirA) enzyme (Avidty, Bulk BirA) following the manufacturer's protocol. All modified proteins without Avitag used herein were biotinylated via free amines using EZ link Sulfo-NHS-LC-Biotin (Thermo/Pierce, 21335) following manufacturer protocols. Table 5 is the list of biotinylated proteins used.

TABLE 5

Reagent
Biotinylated EGF (Thermo)
Avitag-Human ASGPR
Avitag_IL-33-01 (reduced IL-33)
Avitag_IL-33-01 (oxidised IL-33)
Avitag_IL-33-16
HMGB1

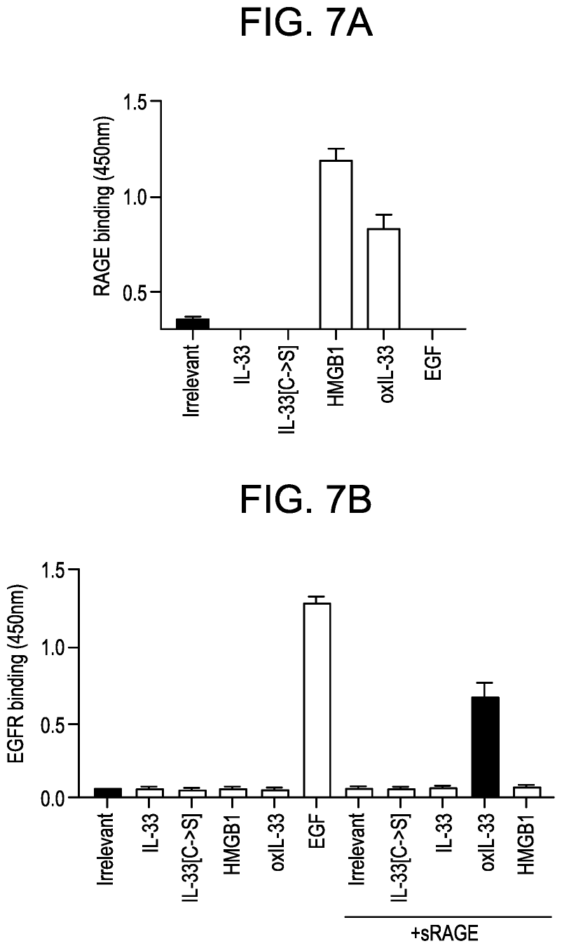

Streptavidin plates (Thermo Scientific, AB-1226) were coated with 100 μl/well of biotinylated antigen (10 μg/ml in PBS) at room temperature for 1 hour. Plates were washed 3× with 200 μl PBS-T (PBS+1% (v/v) Tween-20) and blocked with 300 μl/well blocking buffer (PBS with 1% BSA (Sigma, A9576)) for 1 hour. Plates were washed 3× with PBS-T. RAGE-Fc (R&D Systems #1145-RG) or ST2-Fc (R&D Systems #523-ST) were diluted to 10 μg/mL in PBS in blocking buffer, added to the relevant wells and incubated at room temperature for 1 hour. Alternatively, 100 μl of EGFR-Fc (R&D Systems #344-ER-050) at 10 μg/mL in PBS was added in the presence or absence of untagged RAGE (Sino Biological, 11629-HCCH) at 10 μg/mL in PBS for 1 hour. Plates were washed with 200 μl PBS-T three times. Then RAGE-Fc, ST2-Fc and EGFR-Fc were detected with anti-human IgG HRP (Sigma A0170, 5.1 mg/mL) diluted 1:10000 in blocking buffer, 100 μl/well for 1 hour at room temperature. Plates were washed 3× with PBS-T and developed with TMB, 100 μl/well (Sigma, T0440). The reaction was quenched with 50 μl/well 0.1M H₂SO₄. Absorbance was read at 450 nm on the Cytation Gen5 or similar equipment. The results show that oxIL-33 displayed a clear interaction with RAGE (FIG. 7A) whereas direct binding of oxIL-33 to EGFR was negligible (FIG. 7B). EGFR binding to oxIL-33 was observed only by the addition of sRAGE to this assay (FIG. 7B). This could not be recapitulated if oxIL-33 was substituted for a bona fide RAGE agonist, HMGB1 (FIG. 7B).

The need of RAGE in EGFR signaling triggered by oxIL-33 was further confirmed making use of RAGE-deficient cell lines. A RAGE knockout A549 cell line was generated as follows:

A mammalian plasmid was generated containing expression vectors for red fluorescent protein (RFP), guide RNA targeted to Exon 3 of AGER (TGAGGGGATTTTCCGGTGC SEQ ID NO:47) and Cas9 endonuclease. A549 conditioned media was generated by growing A549 cells in F12K nut mix (Gibco, supplemented with 10% FBS and 1% Penicillin/Streptomycin) in T-175 flasks for two days. Spent media was taken off the A549s, filtered, and diluted five-fold in fresh Gibco F12K nut mix (supplemented with 20% FBS and 1% Penicillin/Streptomycin). A549s were seeded into three T-75 flasks at 2×10⁵ cells/ml in 15 ml total and placed in a 37° C., 5% CO₂ incubator overnight. Transfection mix was prepared using 1.6 ml of F12K nut mix (supplemented with 1% Penicillin/Streptomycin) with 8 μg of the AGER guide RNA plasmid and 22.5 μg PEI (Polysciences, 23966-2). The mix was then vortexed for 10 seconds and left at room temperature for 15 mins. 0.75 ml of the transfection mix was then added to each T-75 flask. The flasks were returned to the incubator for two days. The A549 cells were then detached using Accutase and transferred into PBS containing 1% FBS and single cell sorted on an Aria cell sorter (BD) based on expression of RFP into a 96-well dish. The cells were fed every 3-5 days with conditioned media. Once cells became over 50% confluent, they were transferred to 24-well plates and grown up. This process of upscaling continued until each successful clone was split into T15 flasks. Cells were then split into 12 well plates and grown until over 50% confluent before analysis genomic PCR for successful knockouts. Cells were lysed in 100 μl DNA lysis buffer (Viagen Bitoech, 301-C, supplemented with 0.3 pg/ml proteinase K) per well. These samples were incubated at 55° C. for 4 hours followed by 15 min at 85° C. PCR of RAGE was performed with forward and reverse primers having the following sequences: forward—gttgcagcctcccaacttc (SEQ ID NO:48), reverse—aatgaggccagtggaagtca (SEQ ID NO:49). The reaction and cycling was set up as follows in a 50 μl reaction volume [25 μl Q5 polymerase mix, 2.5 μl forward primer (10 pM stock), 2.5 μl reverse primer (10 pM stock), 2 μl of template DNA lysate, 18 μl nuclease-free water]. The PCR reaction was run with initial denaturation at 98° C. for 30 seconds, followed by 35 cycles of 98° C. for 5 seconds, 57° C. for 10 seconds and 72° C. for 20 seconds before a final step at 72° C. for 2 minutes. 4 μl of the PCR product was mixed with 6 μl of nuclease-free water and 2 μl of 6×DNA loading buffer (Thermo Scientific, R0611). Samples were run on a 1% agarose gel (1:10000 SYBR safe) at 90V for 1 hour before visualisation on Versadoc Imager. The remainder of the PCR products were then cleaned up with the QIAquick PCR purification kit (Qiagen, 28104), following the manufacturer's protocol. DNA-50 concentration was measured using a nanodrop. Several clones (selected from results) were sent for in-house sequencing. Results showed successful insertion of stop codon in clones RAGE09 and RAGE10.

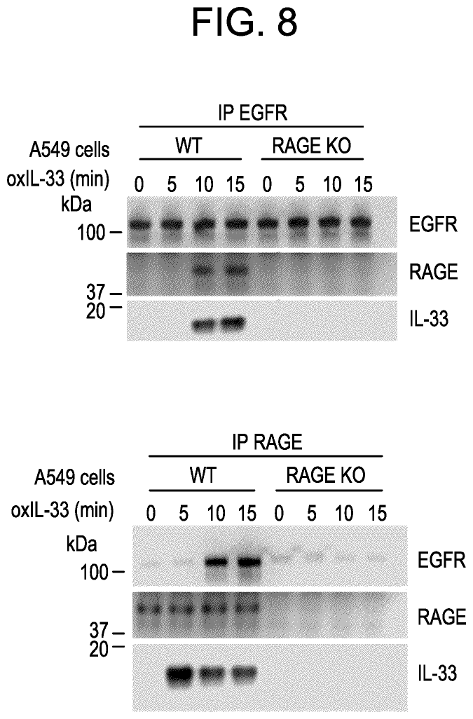

In order to ascertain the essentiality of RAGE to oxIL-33-mediated EGFR signaling, immunoprecipitation and Western blotting were then performed on A549 and the RAGE-deficient A549 cells. Briefly, cell lines were activated at various time points (0-15 minutes) with oxIL-33-01. Subsequent immunoprecipitation of EGFR or RAGE was followed by western blotting with anti-RAGE, anti-EGFR and anti-IL-33 following the relevant experimental protocols detailed in section 9. The results show the essential role of RAGE in the formation of a complex with oxIL-33 and EGFR (FIG. 8)

11. Oxidised IL-33 Induces STAT5 Phosphorylation which is Blocked by RAGE, but not ST2 Neutralizing Antibody

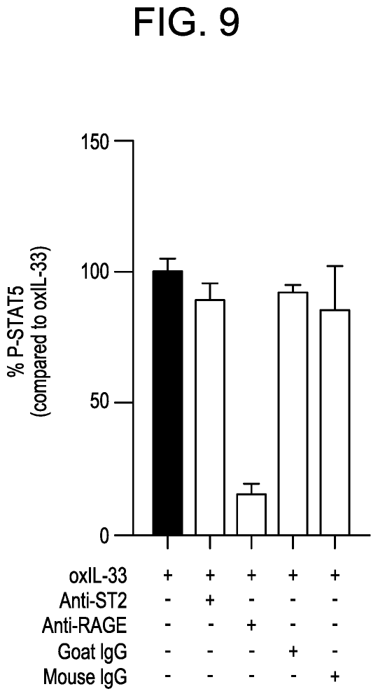

To confirm the importance of RAGE over ST2 in oxIL-33 signaling, blocking antibodies were tested. Briefly, A549s were cultured in RPMI GlutaMax medium supplemented with 1% Penicillin/Streptomycin and 10% FBS. Cells were harvested with accutase and seeded into 96 well plates at 5×10⁵/100 μl and incubated at 37° C., 5% CO₂ for 18-24 hours. The wells were then washed twice with 100 μl of PBS before addition of 100 μl of starve media (RPMI GlutaMax medium supplemented with 1% Penicillin/Streptomycin) and incubated at 37° C., 5% CO₂ for 18-24 hours. Anti-RAGE (M4F4; WO 2008137552); Anti-ST2 (AF532; RnD Systems) or isotype control (MAB002, R&D Systems) was added in a dose dependent manner to the wells and the plate was returned to the incubator for 30 mins. The plates were then stimulated with oxidised IL-33 (30 ng/ml) for 30 mins before lysis using the phosho-STAT5 ELISA kit lysis buffer (85-86112-11, ThermoFisher Scientific) and developed following manufacturer's instructions before reading absorbance at 450 nM. As shown in FIG. 9, cells activated with oxIL-33-01 display phosphorylation of STAT5 which decreased in the presence of anti-RAGE but not anti-ST2 antibody (FIG. 9).

### Example 3—OxIL-33 Triggers the Internalization of EGFR in Epithelial Cells

It was next investigated whether oxIL-33 induces changes in the dynamics of EGFR as compared to EGF.

12. Confocal Experiments of EGF Internalization

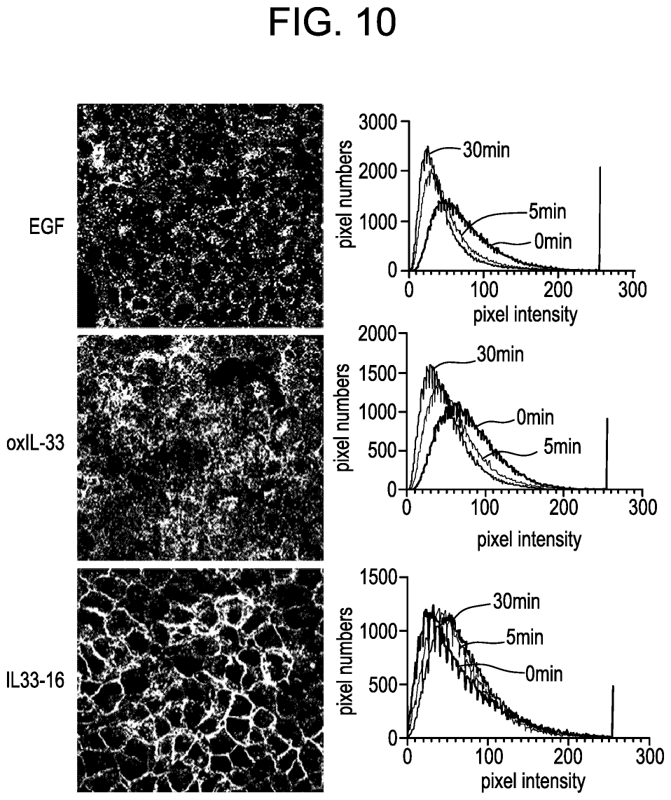

This experiment aims to investigate the dynamics of EGFR in epithelial cells after stimulation with EGF, reduced or oxidised forms of IL-33 utilizing confocal imaging. EGFR-GFP A549 epithelial cell line (Sigma, CLL1141-1VL) were plated at concentration of 20000 cells/ml (RPMI medium+10% FCS+Pen/Strep), 1 ml per 24 well glass bottom plate (Greiner, 662892). The EGF receptor linked to Green Fluorescence Protein (GFP) allows EGFR membrane dynamics and internalisation to be tracked. Cells were washed once with PBS and incubated in RPMI media (without FCS). After 24 hours of starvation, cells were washed with RPMI and incubated with 0.5 ml of RPMI media with CellMask (Invitrogen C10046) deep red at 1:5000 dilution. Cells were stained briefly with CellMask before treatment for membrane marking and live imaged immediately at treatment on confocal at 1 frame/min, to record the dynamics of EGFR-GFP. Cells were stained at 37° C. for 5 minutes, washed once with PBS and stimulated with oxIL-33 (oxidised IL-33-01) or IL-33-16 at a concentration of 200 ng/ml in 0.5 ml serum free RPMI/well. Confocal images were taken immediately, 40× oil objective, 1 min/frame, 5 stacks spacing 2 μm for 25 minutes (about 30 minutes after adding the proteins). Disrupted (dotted) pattern of the GFP signal indicates clustering of the receptor on the membrane and internalisation. Pixel intensity histograms of the membrane area (masked by CellMask) and the intracellular area (masked by inverted CellMask, not shown) were generated at different time points from live imaging, showing depletion of EGFR in non-clustered area (left shift of the histogram bell shape peaks), and increased numbers of saturated pixels (intensity 255) caused by clustering. oxIL-33 induced clustering and internalization of EGF receptor, although EGF stimulation resulted in most evident EGFR clustering. In contrast, reduced form of IL-33 (IL-33-16) did not display major changes in EGFR cell distribution (FIG. 10).

### Example 4—OxIL-33 Induces the Secretion of IL-8 by Epithelial Cells, Similar to EGF

13. Selective Secretion of IL-8 by oxIL-33

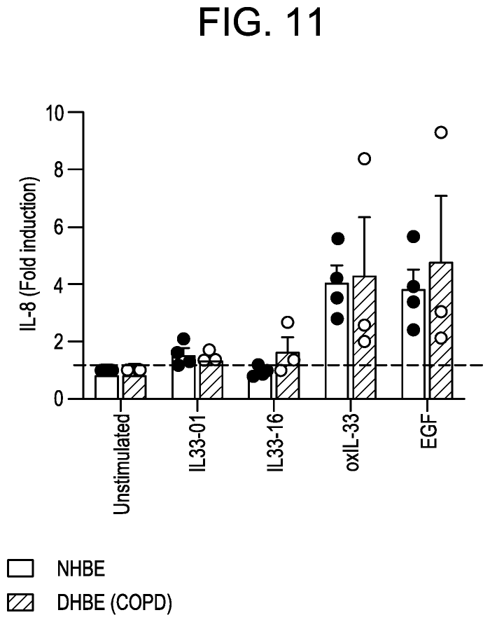

Human bronchial epithelial cells from healthy subjects (NHBE; Lonza CC-2540) and chronic obstructive pulmonary disease (COPD) (DHBE; Lonza 00195275) were maintained in complete BEGM media (Lonza) according to the manufacturers protocol for one month with a media change every three days until the cells reached confluency. Cells were harvested with accutase and seeded at 5×10⁵/100 μl in a 96-well plate (Corning 3596) in culture media. The plates were incubated at 37° C., 5% CO₂ for 18-24 hours. After this time, media was aspirated, and the cells were washed twice with 100 μl PBS before the addition of starve media (BEGM (Lonza CC-3171) without supplement kit supplemented with 1% Penicillin/Streptomycin). The plates were then incubated at 37° C., 5% CO₂ for a further 18-24 hours before stimulation with media alone (unstimulated control), 30 ng/ml reduced IL-33-01, 30 ng/mL IL-33-16, 30 ng/mL oxidised IL-33-01 or 30 ng/mL EGF and returned to 37° C., 5% CO₂. 24 hours after stimulation, supernatant were collected and evaluated for chemokine production using a multiplex assay (Mesoscale Discovery K15047D-2). As shown in FIG. 11, NHBEs and DHBEs display a 4-fold increase in the secretion of IL-8 upon activation with oxIL-33 as compared to unstimulated cells (media alone). No major differences were observed for other chemokines (TARC, MIP-1a, MIP1b, MCP4, MCP1, IP10, Eotaxin, Eotaxin-3, MDC—data not shown).

### Example 5—OxIL-33 Impairs Scratch Wound Repair Response in Submerged Monolayer Epithelial Cultures

14. oxIL-33 Impairs Scratch Wound Closure in A549 and NHBE Cells, in Contrast to EGF

A549s were obtained from ATCC and cultured in RPMI GlutaMax medium supplemented with 1% Penicillin/Streptomycin and 10% FBS. Cells were harvested with accutase (PAA, #L11-007) and seeded into 96 well plates at 5×10⁵/100 μl and incubated at 37° C., 5% CO₂ for 18-24 hours. The wells were then washed twice with 100 μl of PBS before addition of 100 μl of starve media (RPMI GlutaMax medium supplemented with 1% Penicillin/Streptomycin) and incubated at 37° C., 5% CO₂ for 18-24 hours. Using a WoundMaker™ (Essen Bioscience), cells were scratched and then wells were washed 2× with 200 μl of PBS before addition of RPMI GlutaMax medium supplemented with 0.1% FBS (v/v) and 1% (v/v) Penicillin/Streptomycin containing the indicated stimulations; media alone (unstimulated control), 30 ng/ml reduced IL-33-01, 30 ng/mL oxidised IL-33-01 or 30 ng/mL EGF and returned to 37° C., 5% CO₂. Plates were placed into an IncucyteZoom for wound healing imaging and analysis over a 48 hour period. Relative Wound Density was calculated through the wound healing algorithm within the Incucyte Zoom software.

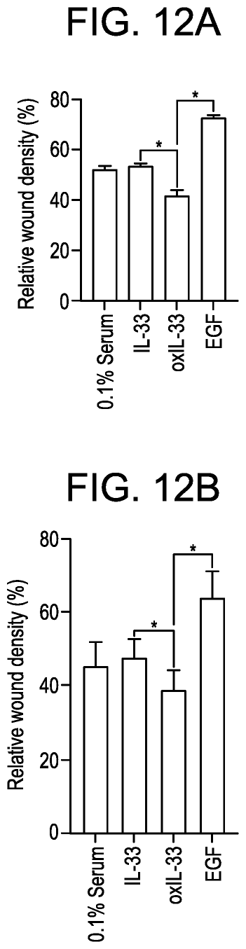

NHBEs (CC-2540) were obtained from Lonza and were maintained in complete BEGM media [BEGM (Lonza CC-3171) and supplement kit (Lonza CC-4175)] according to the manufacturer's protocol. Cells were harvested with accutase and seeded at 5×10⁵/100 μl in a 96-well ImageLock plate (Sartorius, 4379) in culture media. The plates were incubated at 37° C., 5% CO₂ for 18-24 hours. After this time, media was aspirated, and the cells were washed twice with 100 μl PBS before the addition of starve media (BEGM (Lonza CC-3171) without supplement kit supplemented with 1% Penicillin/Streptomycin). The plates were then incubated at 37° C., 5% CO₂ for a further 18-24 hours before scratch wounding. Using a WoundMaker™ (Essen Bioscience), cells were scratched and then wells were washed 2× with 200 μl of PBS before addition of BEBM media (Lonza) supplemented with 0.1% FBS (v/v) and 1% (v/v) Penicillin/Streptomycin containing the indicated stimulations; media alone (unstimulated control), 30 ng/ml reduced IL-33-01, 30 ng/mL oxidised IL-33-01 or 30 ng/mL EGF and returned to 37° C., 5% CO₂. Plates were placed into an IncucyteZoom for wound healing imaging and analysis over a 48 hour period. Relative Wound Density was calculated through the wound healing algorithm within the Incucyte Zoom software. As shown in FIG. 12, oxIL-33 inhibited wound healing in submerged cultures of A549 cells (FIG. 12A) and NHBE cells (FIG. 12B), having an opposite effect to EGF where increased wound cell density is observed.

15. The Impairment of Scratch Wound Closure by Oxidised IL-33 can be Prevented by Antibodies Neutralising RAGE or EGFR but not ST2

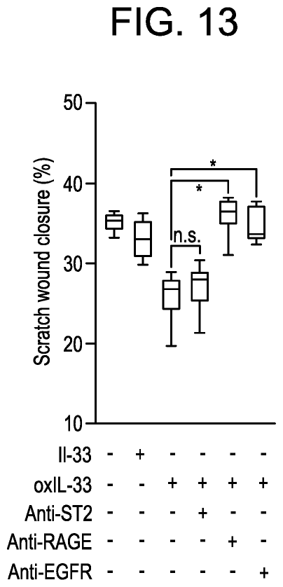

To understand whether these functional effects of oxIL-33 were mediated through RAGE/EGFR, the scratch assay was performed in NHBE cells as described in section 14, but in the presence of antibodies that neutralised different receptor components. NHBE cells were treated with media alone (unstimulated control), reduced IL-33, or oxidised IL-33, in the presence of 10 μg/mL anti-ST2 (AF532, R&D Systems), anti-RAGE (M4F4, WO 2008137552) or anti-EGFR (Clone LA1, 05-101 Millipore). OxIL-33, but not reduced IL-33, inhibits scratch closure. This effect of oxIL-33 is reversed by anti-RAGE and anti-EGFR but not anti-ST2, again demonstrating that RAGE and EGFR are essential receptors involved in the oxidised IL-33 signalling pathway (FIG. 13).

### Example 6—Anti-IL-33 Improves the Phenotype of COPD Cells in Submerged Cultures

16. OxIL-33 can Drive a COPD-Like Response in Healthy NHBEs in a Scratch Wound Closure Assay

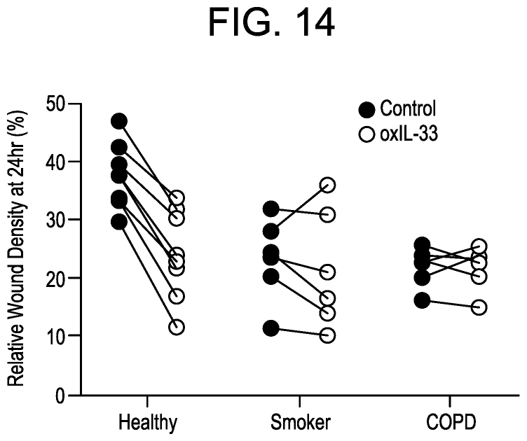

Next, the effect of oxidised IL-33 in healthy, smokers and COPD bronchial epithelial cells was investigated. NHBEs (CC-2540), NHBEs from smokers (CC-2540) and DHBEs (COPD, 00195275) were obtained from Lonza and were maintained in complete BEGM media (Lonza) according to the manufacturers protocol. A scratch assay was performed as described in Section 14. Cells were treated with media alone (unstimulated control), or 30 ng/mL oxidised IL-33. Bronchial epithelial cells from smokers or COPD showed an impaired ability for scratch closure compared with cells from healthy subjects that was similar to the impairment observed after treatment of healthy cells with oxIL-33 (FIG. 14). In contrast with healthy cells, scratch closure response was not further impaired by oxIL-33 in smoker and COPD HBE cells (FIG. 14).

17. Blockade of Endogenous IL-33 Through the RAGE/EGFR Pathway can Improve the Impaired Scratch Wound Repair Phenotype of COPD Basal Cells.

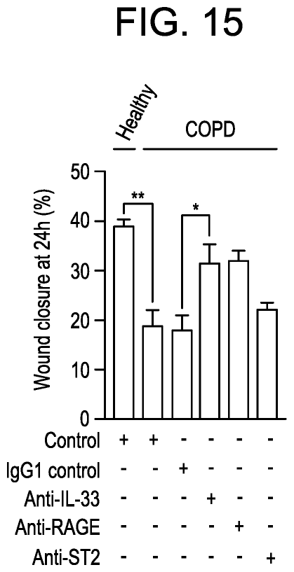

Since epithelial cells are known to produce IL-33, it was possible that autocrine IL-33 secretion could account for the impaired scratch repair phenotype observed in the COPD cells. To investigate, a scratch closure assay was performed in bronchial epithelial cells from COPD in the presence of IL-33 neutralisation. NHBEs (Lonza CC-2540) and DHBEs (Lonza, COPD 00195275) were maintained in complete BEGM media (Lonza) according to the manufacturers protocol. Cells were harvested with accutase and seeded at 5×10⁵/100 μl in a 96-well ImageLock plate (Sartorius, 4379) in culture media. The plates were incubated at 37° C., 5% CO₂ for 18-24 hours. After this time, media was aspirated, and the cells were washed twice with 100 μl PBS before the addition of starve media (BEGM (Lonza CC-3171) without supplement kit supplemented with 1% Penicillin/Streptomycin). The plates were then incubated at 37° C., 5% CO₂ for a further 18-24 hours before scratch wounding. Using a WoundMaker™ (Essen Bioscience), cells were scratched and then wells were washed 2× with 200 μl of PBS before addition of BEBM media (Lonza) supplemented with 0.1% FBS (v/v) and 1% (v/v) Penicillin/Streptomycin containing 10 μg/mL of anti-IL-33 (33_640087-7B, described in WO2016/156440), anti-ST2 (AF532, R&D Systems), anti-RAGE (M4F4, WO 2008137552) or NIP228 (IgG1 isotype control), and returned to 37° C., 5% CO₂. Plates were placed into an IncucyteZoom for wound healing imaging and analysis over a 48 hour period. Relative Wound Density was calculated through the wound healing algorithm within the Incucyte Zoom software. As observed previously, COPD cells were impaired in their scratch closure response compared with cells derived from healthy subjects. Anti-IL-33 and anti-RAGE, but not anti-ST2, were able to improve the scratch closure response of the COPD cells to a level similar to healthy cells (FIG. 15) demonstrating that epithelial cells produce autocrine IL-33 that signals through the RAGE/EGFR pathway (FIG. 15).

### Example 7—Anti-IL-33 Reduces Goblet Cells in 3D Epithelial Cultures

18. Air-Liquid Interface (ALI) Culture of Airway Basal Cells

Next the inventors sought to determine the relevance of IL-33 signalling in Air-liquid interface cell cultures (“ALI cultures”). ALI culturing is a method by which basal cells are grown with their basal surfaces in contact with media and the top (apical) cellular layer exposed to the air. ALI culturing enables the development of a three-dimensional cellular structure in vitro with the mucociliary phenotype of a pseudostratified epithelium, similar to the tracheal epithelium. ALI cultures can therefore be used to study fundamental aspects of the respiratory epithelium, such as cell-to-cell signalling, disease modelling, and respiratory regeneration.

Cryovials of frozen lung basal cells from healthy controls or COPD patients were received from the University of North Carolina and the University of Pittsburgh. Cells were thawed and plated on a T-75 flask coated with Purecol Type I Bovine Collagen (Advanced BioMatrix, San Diego, Calif.) diluted 1:70 in 1×PBS (Gibco, Waltham, Mass.) and grown in Epix Media (276-201, Propagenix, Rockville, Md.). After reaching confluency, these cells were split once to an appropriate number of T-75 flasks before being harvested for ALI culture. Transwells for ALI culture containing 12 mm, 0.4 μM polyester membrane inserts (Costar, Corning, N.Y.) were prepared by coating the inserts with 1:70 Purecol solution and incubating at 37° C. for between 1-16 hours. The Purecol solution was removed and the Transwells were placed under a UV light for 30 minutes and then washed with PBS. The basal cells in T-75 flasks were detached using 4 ml of trypsin solution (ThermoFisher, 15400054). The cell suspension was added to a 50 ml tube containing 5 ml of FBS and then counted on a ViCell counter (Beckman Coulter, Brea, Calif.) and spun down at 1,000 RPM for 5 minutes. The cells were then resuspended in Pneumacult ALI media (Stemcell Tech, Vancouver, BC) at a density of 3.57×10⁵/ml and 700 μl was dispensed onto each Transwell. 1 mL of ALI media was added into the space below the insert. Cells were left submerged in ALI media until confluent and tight junctions are formed (typically 7 days), at which point the media was removed from the apical side and cells were differentiated for 2 weeks, with media change on the basal side every other day. Fully differentiated cultures were treated with no antibody, 1 mg/ml anti-IL-33 (33_640087-7B) or 1 mg/ml NIP228 (IgG1 isotype control) for 7 days by inclusion of treatments in the media supplied to the basal side of the culture. A media change was performed every other day (containing relevant treatments).

19. IHC Triplex Staining (Basal, Goblet and Ciliated) and Quantitation

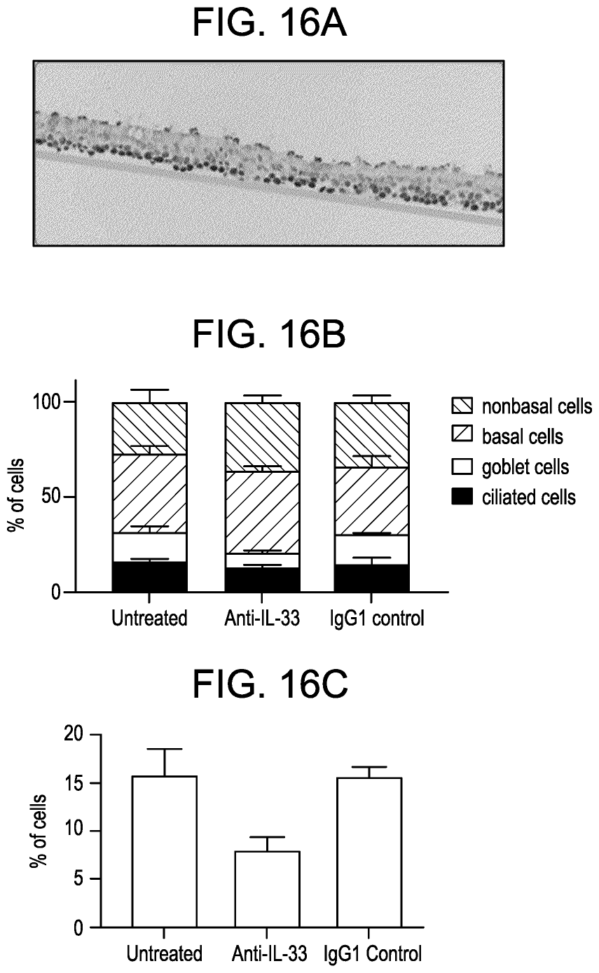

ALI cultures from COPD donors were generated and treated as described in section 18. ALI epithelial cultures were fixed in 10% neutral buffered formalin for 24-hours and embedded in paraffin. Paraffin sections (4 um) were mounted on positively charged slides and stained on the Ventana Discovery Ultra with a sequential 3 plex chromogenic assay. Antigen retrieval was done with cell conditioner 1 (CC1) (cat #5424569001, Roche) and endogenous peroxidase was blocked with Discovery Inhibitor (cat #7017944001, Roche) for 12 min. Anti-p63 (clone 4A4) (cat #790-4509, Roche, Basel, Switzerland) was applied for 24 min at 36° C. and visualized with mouse anti-HQ (12 min) (cat #7017782001, Roche) and anti-HQ HRP (12 min) (cat #7017936001, Roche), and incubated in the Teal substrate (cat #8254338001, Roche) for 12 min. The slides were treated with an antibody denature step (100° C. for 24 min) with cell conditioner 2 (CC2) (cat #5424542001, Roche) and then anti-tubulin (cat # ab24610, Abcam, Cambridge, UK) diluted 0.01 μg/ml with Dako Antibody Diluent (cat # S3022) for 16 min and detected with mouse OmniMap-HRP (8 min) (cat #5269652001, Roche) and visualized with Discovery Purple substrate (cat #7053983001, Roche) for 16 min. The slides were subjected to an additional antibody denaturation with CC2 and then a cocktail of rabbit anti-Mucin 5AC 1.1 μg/ml and rabbit anti-Mucin 5B 7 μg/ml (cat # ab198294 and cat # ab87376, Abcam respectively), were applied for 20 min and visualized with anti-rabbit NP (4 min) (cat #7425317001, Roche), anti-NP-AP (8 min) (cat #7425325001, Roche) and then Discovery Yellow (cat #7698445001, Roche) for 20 min. The stained slides were rinsed with Dawn detergent, counterstained with hematoxylin (cat #5277965001, Roche), rinsed, dehydrated with graded series of ethanol and xylene and mounted with permanent mounting media. Quantification using HALO software showed a decrease in goblet cells in ALI culture derived from healthy donors that had been treated with anti-IL-33 (FIG. 16).

### Example 8—Anti-IL-33 Regulates Mucins in 3D Epithelial Cultures from COPD and Improves Mucus Movement

20. IHC Duplex IF Staining (Mucin5B+Mucin5AC)

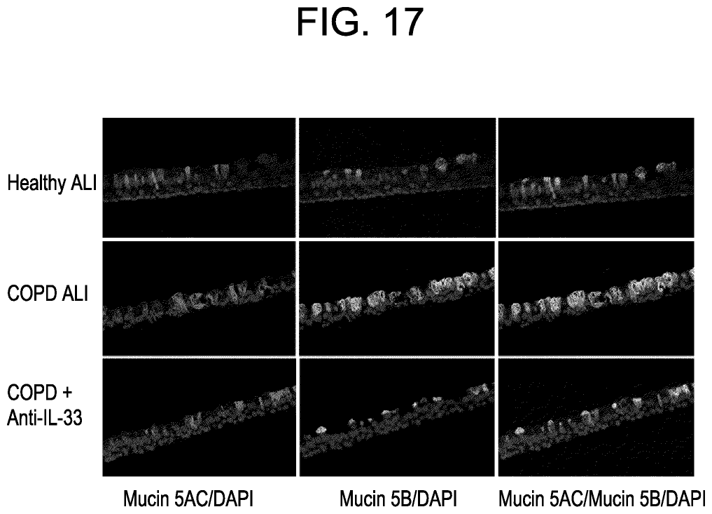

ALI cultures from COPD donors were generated and treated as described in section 18. ALI epithelial cultures were fixed in 10% neutral buffered formalin for 24-hours and embedded in paraffin. Paraffin sections (4 um) were mounted on positively charged slides and stained on the Ventana Discovery Ultra with a sequential 2 plex immunofluorescent assay. Antigen retrieval was done with cell conditioner 1 (CC1) and endogenous peroxidase was blocked with Discovery Inhibitor for 12 min and the blocked 8 min with S Block (RUO) Roche Diagnostics (cat #760-4212) and incubated in anti-Mucin5B used at 7 μg/ml diluted in Dako Ab Diluent, S3022, for 24 min at 36C, and detected with anti-rabbit-HQ (Roche Diagnostics Cat #760-4815) for 4 min and anti-HQ-HRP (Roche Diagnostics cat #760-4820) for 8 min. The samples were then incubated with Discovery FITC, a tyramide conjugate, (Roche Diagnostics cat #760-232) for 8 min. Dual Sequence was selected in the Discovery Ultra program and the samples were treated with an antibody denature step, (100° C. for 24 min) with cell conditioner 2 (CC2) and then neutralized with Discovery Inhibitor (40C, 24 min) before application of anti-Mucin5AC, 1.1 μg/ml, for 20 min at 36° C. The Mucin5AC was detected with anti-rabbit-HQ (Roche Diagnostics Cat #760-4815) for 4 min and anti-HQ-HRP (Roche Diagnostics cat #760-4820) for 8 min, and visualized with Discovery Red610, a tyramide conjugate, for 8 min. After completion of this step, the stained slides were removed from the Discovery Ultra Autostainer and were rinsed with Dawn detergent and then de-ionized water. The samples were the incubated in 4′,6-diamidino-2-phenylindole, dihydrochloride (DAPI Nucleic Acid Stain), ThermoFisher cat # D1306, diluted in de-ionized water at 1 μg/ml for 2 min. Samples were rinsed with de-ionized water and coverslipped with ProLong Gold Antifade mounting media (ThermoFisher, cat # P36930) and stored in light tight slide box. Stained slides were imaged with Zeiss LSM 880 confocal microscope (Carl Zeiss Microscopy, LLC, White Plains, N.Y.). FIG. 17 shows that anti-IL-33 treatment of ALI cultures may lead to the down-regulation of different mucins in COPD cultures

21. Anti-IL-33 Reverses the Impaired Mucociliary Clearance Observed in COPD ALI Cultures

ALI cultures from COPD donors were generated and treated as described in section 18. 30 μl of 0.2 μM FluoSpheres (ThermoFisher, F8811) diluted 1:33 in PBS was then added to the apical surface and using a Zeiss LSM800 microscope a short video of the FluoSphere movement was captured and shows that mucociliary movement increases after treatment with anti-IL-33 (33_640087-7B) but not control antibody.

### Example 9—Single Cell RNA Analysis of ALI Cultures Shows Goblet Cell Changes after Treatment with Anti-IL-33

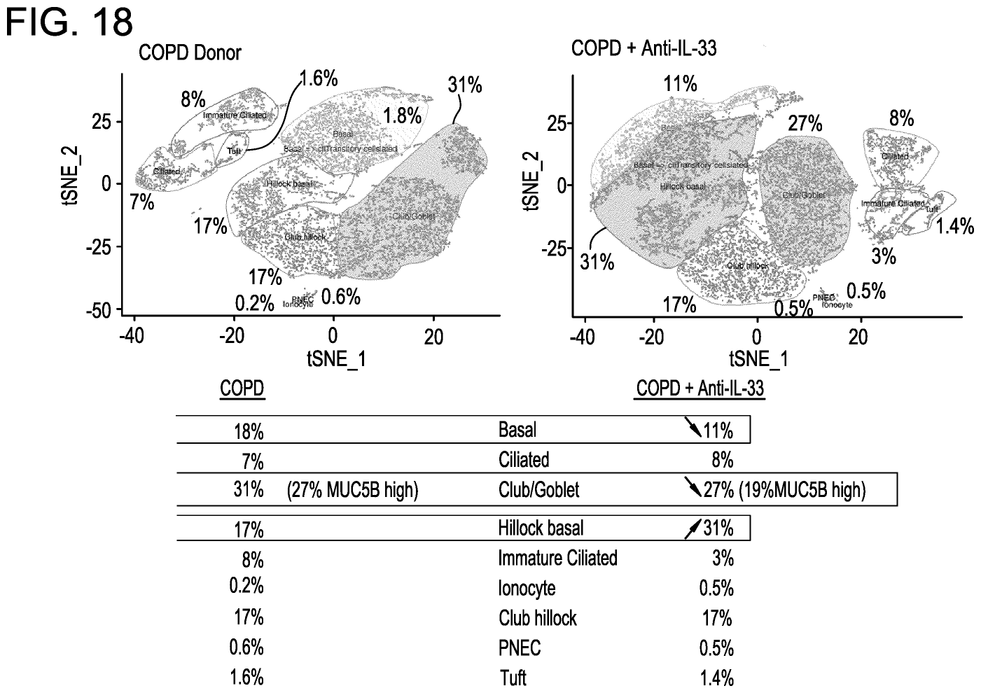

ALI cultures from COPD donors were generated and treated as described in section 18. To obtain a single-cell suspension, the filter insert was incubated with 0.25% trypsin for 5 min at 37° C. degrees. Epithelial cells were gently detached from the filter by washing with PBS pipetting up and down and then transferred to a 15 ml Falcon tube. Cells were centrifuged at 1000 RPM for 5 min at 4° C. After removing the supernatant, the cells were resuspended in 0.4% BSA in PBS and the cell concentration was adjusted to 1000 cells/μl for sequencing. Cell suspension was loaded according to the standard protocol in Chromium single cell 3′ kit to capture between 5000 and 10.000 cells/channel. Version 2 chemistry was used. Single cell 3′ libraries for Illumina sequencing were obtained followed the manufacturer's protocol (Chromium™ Single Cell 3′ Reagent Kit, v2 Chemistry). Libraries were assessed for quality (TapeStation 4200, Agilent) and then sequenced on NextSeq 500 or NovaSeq 6000 instruments (Illumina). Initial data processing was performed using the Cell Ranger version 2.0 pipeline (10× Genomics). Post-processing was performed using the Seurat package for exclusion of low cell quality and normalization. Each sample was analyzed as independent data to capture within-sample heterogeneity (cell subtyping). Clustering and visualization were realized with t-Distributed Stochastic Neighbor Embedding (tSNE). Identification of cell clusters in COPD was guided by marker genes. For other samples, initial clusters were inspected manually then Seurat's Label Transfer algorithm was applied for subtype identification. MUC5AC and MUC5B gene expression analysis was performed for each cluster between cells from COPD ALI cultures with and without anti-IL-33 treatment as mentioned in section 18. Heatmaps and tSNE plots were generated using Seurat. FIG. 18 shows tSNE plots illustrating the different proportions of cell subtypes that are found in ALI cultures treated with anti-IL-33 (33_640087-7B) as compared to no treatment. As shown in FIG. 18, a decrease in MUC5B high cells was noted after anti-IL-33 treatment.

### Example 10—Anti-IL-33 Reduces Goblet Cells in COPD 3D Epithelial Cultures

22. Air Liquid Interface (ALI) Culture of Airway Basal Cells

In order to quantify and interrogate the effects of oxIL-33 in a physiologically relevant Air liquid interface (ALI) culture system, a flow cytometry assay aiming to discriminate between goblet cell types (MUC5ac vs MUC5b) and the rest of epithelial populations (Mucin negative) was developed.

Cryovials of frozen lung basal cells from healthy (CC-2540) controls or COPD (195275) patients were received from Lonza. One vial per donor was thawed and plated on 4×T-175 flasks in Epix Media (276-201, Propagenix, Rockville, Md.). After reaching confluency, these cells were frozen down at 1e6 cells per vial at P2. Cells at P2 were initiated into 2×T-75 flasks in Epix Media and grown until 80% confluent. Transwells for ALI culture containing 12 mm or 6.5 mm 0.4 μM polyester membrane inserts (Costar, Corning, N.Y.) were prepared by coating the inserts with 1× Collagen I solution (Celladhere™ Collagen I—Stemcell #07001, prepared in dH2O) and incubating at 37° C. for between 1-16 hours. The Collagen I solution was removed and the Transwells were washed with PBS. The basal cells in T-75 flasks were washed with PBS and detached using 6 ml of trypsin solution (Lonza trypsin subculture pack—#CC-5034). The trypsin was neutralised with 6 ml of trypsin neutralising solution (Lonza trypsin subculture pack—#CC-5034) and the cell suspension was added to a 15 ml tube, counted and tube spun down at 1,200 RPM for 5 minutes. The cells were then resuspended in Pneumacult ALI media (Stemcell Tech, Vancouver, BC) at a density of 8×10⁵/ml and 0.5 ml was dispensed onto each 12 mm Transwell and 0.25 ml onto each 6.5 mm Transwell. 1 mL of ALI media was added into the space below the 12 ml insert and 0.5 ml below the 6.5 mm insert. Cells were left submerged in ALI media until confluent and tight junctions are formed (typically 7 days), at which point the media was removed from the apical side and cells were differentiated for 3 weeks, with media change on the basal side every Monday, Wednesday and Friday. Fully differentiated normal cultures were left untreated or treated with reduced or oxidised untagged IL33-01 (30 ng/ml), untagged IL33-16 (30 ng/ml), IL-13 (10 ng/ml), EGF (30 ng/ml) or HMGB1 (30 ng/ml) for 7 days by inclusion of treatments in the media supplied to the basal side of the culture (7 day treatments). Fully differentiated COPD cultures were left untreated or treated with 1 μg/ml anti-IL-33 (33_640087-7B), 1 μg/ml NIP228 (IgG1 isotype control), 10 μg/ml mNIP228, 10 μg/ml anti-ST2, 1 μg/ml anti-RAGE or 1 μg/ml anti-EGFR for 7 days by inclusion of treatments in the media supplied to the basal side of the culture. A media change was performed every Monday, Wednesday and Friday (containing relevant treatments).

TABLE 6

| Antibody | Identifier |
|---|---|
| hIgG1 | NIP228_SP14-266 |
| anti-IL-33 (33_640087-7B) | SP15-124 |
| mIgG1 | mNIP228_SP14-108 |
| Anti-ST2 | Ab1440361 |
| Anti-RAGE | M4F4 |
| Anti-EGFR | LA1 (Merk, 05-101) |

23. FACS Analysis of Goblet Cells in ALI Cultures

Following a 7-day treatments (Table 6), 4-week old normal control or COPD ALI cultures on 6.5 mm inserts were analysed by flow cytometry. 200 μl 37° C. PBS was added to the apical region (Transwell surface) of each Transwell and placed in an incubator for 30 min. The apical wash was stored at −80 for mucin analysis. 150 μl trypsin (Lonza trypsin subculture pack—#CC-5034) was added onto both the apical and basolateral (beneath Transwell) compartments. Transwells were returned to the incubator for 30 mins. The ALIs were dissociated by gently pipetting the trypsin up and down. 150 μl trypsin neutralising solution (Lonza trypsin subculture pack—#CC-5034) was added to each apical chamber and mixed. The cell suspension was moved to U-shaped 90-well plate, cells were counted and centrifuged at 1200 RPM at 4° C. for 5 min. The trypsin/TNS was removed and 200 μl of live dead stain (eBioscience™ Fixable Viability Dye eFluor™ 780 Thermo 65-0865-14, dilute 1:2000 in PBS) was added to each well. The cells were re-suspended and incubated for 10 min on ice in the dark. The plate was centrifuged at 1200 RPM at 4° C. for 5 min, the live dead stain was removed and 200 μl PBS was added to each well. The plate was centrifuged at 1200 RPM at 4° C. for 5 min and the PBS was removed and replaced with 200 μl fixation/permeabilization solution (Thermo 00-5123 and 00-5223). The plate incubated for 40 min on ice in the dark. The plate was centrifuged at 1200 RPM at 4° C. for 5 min and the solution was removed. Cells were re-suspend in 300 μl of 1× permeabilization solution (Thermo 00-8333). 5e4 cells from each well were added to a new 96-well U bottom plate centrifuged at 1200 RPM at 4° C. for 5 min and the cells re-suspended in 50 μl of 1× permeabilization solution. 50 μl of antibody stain cocktail (anti-Muc5AC at 1:400 and anti-Muc5B at 1:800) or isotype stain cocktail at the same dilutions. The plate was incubated for 30 mins on ice in the dark. The plate was centrifuged at 1200 RPM at 4° C. for 5 min and the solution was removed. The cells were washed with PBS, centrifuged and then re-suspended in 150 μl of PBS. Data was then acquired on a BD FACSymphony™ and analysed using FlowJo software.

TABLE 7

| Name | Identifier | Supplier | Fluorophore | Cell marker |
|---|---|---|---|---|
| Anti-Muc5AC | ab3649 | Abcam | Coupled to AF488 | Goblet |
| | | | (Expedeon, | |
| | | | 332-0005) | |
| Anti-Muc5B | ab105460 | Abcam | Coupled to AF647 | Goblet |
| | | | (Expedeon, | |
| | | | 336-0005) | |
| AF488 Isotype | 400109 | BioLegend | FITC | Isotype |
| AF647 Isotype | 400130 | BioLegend | AF647 | Isotype |

24. qPCR/Bulk RNA Seq Analysis of ALI Cultures.

Following a 7-day treatment (Table 6), 4-week old normal control or COPD ALI cultures on 6.5 mm inserts were lysed for RNA analysis. First 200 μl 37° C. PBS was added to each ALI apical surface and the plates were returned to an incubator for 30 min. The apical wash was stored at −80 for mucin analysis. The MagMAX™-96 Total RNA Isolation Kit (Thermo, AM1830) was used to lyse the ALI cultures and extract RNA. RNA was then used to synthesise cDNA using the High-Capacity RNA-to-cDNA™ Kit (Thermo, 4388950). Whereby 9 μl of each RNA sample was incubated with 10 μl of 2×RT Buffer Mix and 1 μl of 20×RT Enzyme Mix in PCR Tubes (Thermo, AM12230) and placed on a thermo cycler and incubated at for 37° C. for 60 minutes. The reaction was stopped by heating to 95° C. for 5 minutes and holding at 4° C. 60 μl of nuclease free water (Thermo, 750024) was added to each tube containing 20 μl of cDNA. For RT-qPCR, 4 μl of cDNA was added to a MicroAmp™ EnduraPlate™ Optical 384-Well Clear Reaction Plates with Barcode (Thermo. 4483273) with 5 μl of TaqMan Fast Advanced Master Mix (Thermo, 4444557) and 0.5 μl of Muc5AC FAM probe (Thermo, Hs01365616_m1) and 0.5 μl of GAPDH VIC probe (Thermo, Hs02786624_g1). Plates were sealed and briefly centrifuged before analysis using a QuantStudio™ 7 Flex Real-Time PCR System (Thermo). Delta-delta-ct was then calculated by normalising the data to an untreated normal control.

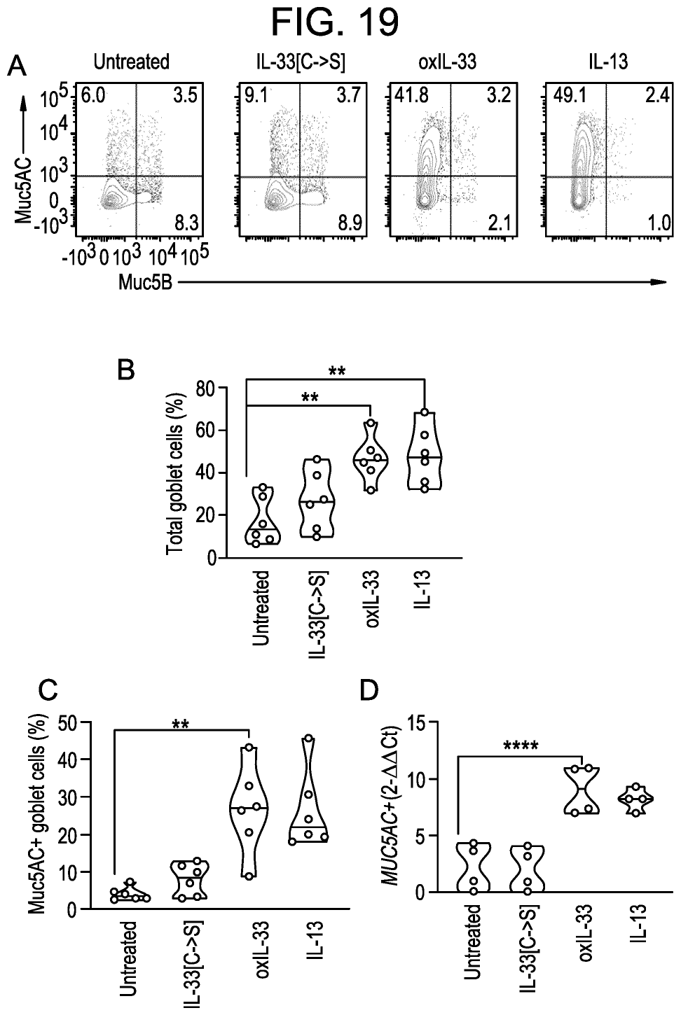

oxIL-33 but not reduced IL-33 leads to increased goblet cell number, in particular the MUC5AC+ goblet cells subset (FIG. 19A-C). Accordantly, MUC5AC mRNA copies were increased upon treatment with oxIL-33 as judged by qPCR (FIG. 19D).

25. IHC Triplex Staining (Basal, Goblet and Ciliated) and Quantitation

Next, quantitative image analysis from ALI immunohistochemistry was evaluated. ALI cultures from COPD donors were generated and treated as described in section 22; Air Liquid Interface (ALI) culture of airway basal cells. ALI epithelial cultures were fixed in 10% neutral buffered formalin for 24-hours and embedded in paraffin. Paraffin sections (4 um) were mounted on positively charged slides and stained on the Ventana Discovery Ultra with a sequential 3 plex chromogenic assay. Antigen retrieval was done with cell conditioner 1 (Ultra CC1) (cat #5424569001, Roche) and endogenous peroxidase was blocked with Discovery Inhibitor (cat #7017944001, Roche) for 12 min. Anti-p63 (clone 4A4) (cat #790-4509, Roche, Basel, Switzerland) was applied for 24 min and visualized with anti-Mouse HQ (12 min) (cat #7017782001, Roche) and anti-HQ HRP (12 min) (cat #7017936001, Roche), and incubated with the Discovery Purple kit (cat #07053983001, Roche) for 12 min. The slides were treated with an antibody denature step (92° C. for 24 min) with cell conditioner 2 (Ultra CC2) (cat #5424542001, Roche) and then anti-tubulin (cat # ab24610, Abcam, Cambridge, UK) diluted with Dako Antibody Diluent (cat # S3022) for 16 min (concentration on slide 0.003 μg/ml) and detected with mouse OmniMap-HRP (8 min) (cat #5269652001, Roche) and visualized with Discovery Teal HRP kit (cat #82544338001, Roche). The slides were subjected to an additional antibody denaturation with CC2 and then a cocktail of rabbit anti-Mucin 5AC 1.1 μg/ml (dispenser concentration) and rabbit anti-Mucin 5B 7 μg/ml (dispenser concentration) (cat # ab198294 and cat # ab87376, Abcam respectively), were applied for 20 min and visualized with anti-rabbit NP (4 min) (cat #7425317001, Roche), anti-NP-AP (8 min) (cat #7425325001, Roche) and then Discovery Yellow kit (cat #7698445001, Roche) for 20 min. The stained slides were counterstained with Hematoxylin II (8 min) (cat #5277965001, Roche) and Bluing reagent (4 min) (cat #5266769001, Roche), rinsed with dish washing detergent, dehydrated with graded series of ethanol and xylene and mounted with permanent mounting media.

IHC images were analysed in HALO v3.1 (Indica Labs), where they were first annotated manually to exclude out of focus and tissue damage areas. A random forest classifier was trained to recognise the epithelium and separate it from transmembrane and glass slide background. For cilia area quantification, another random forest classifier was trained for coarse detection of tubulin staining, followed by fine detection using algorithm Area Quantification v2.1.7. For Mucin area quantification Area Quantification v2.1.7 was directly used to detect the staining. For basal (p63+) cell counting, algorithm CytoNuclear 2.0.9 was used to segment cells based on nuclear staining, basal cells were further detected by counting p63 positive nuclei. All quantification methods were validated against human recognition and had more than 90% accuracy.

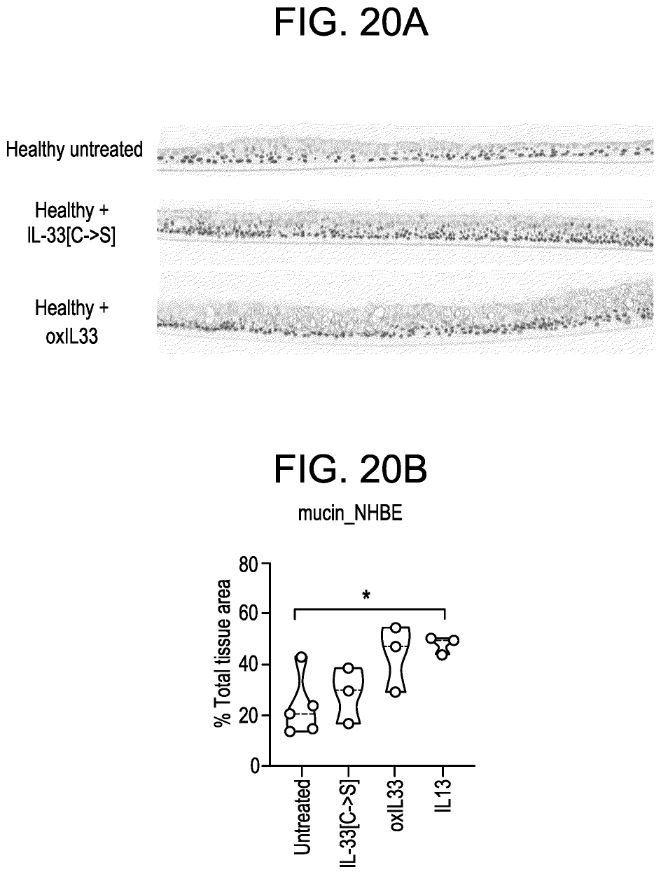

In line with previous findings, oxIL-33 profoundly affected the number of goblet cells (MUC5ac+b) (FIGS. 20A and 20B).

Together, these studies showed a role for oxIL-33 in promoting differentiation of goblet cells within the lung epithelia. This suggest that epithelia chronically exposed to ox-IL33 evolve towards a goblet hyperplasic phenotype that negatively impacts lung function.

26. Reversal of COPD Goblet Phenotype with Blocking Agents

An important hallmark of COPD is the excess of mucus, due to increases in goblet cells and in mucus secretion (Gohy et al 2019 Sci Rep 9:17963). To investigate whether oxidised IL-33 could play a direct role in the goblet COPD phenotype, ALI cultures from COPD donors were established with readouts as described in sections 22-25.

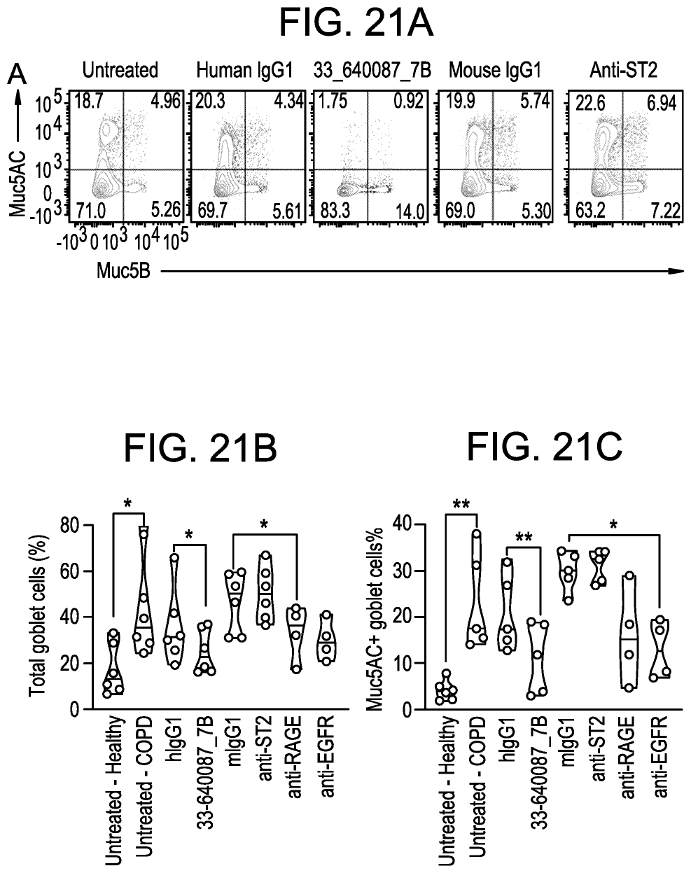

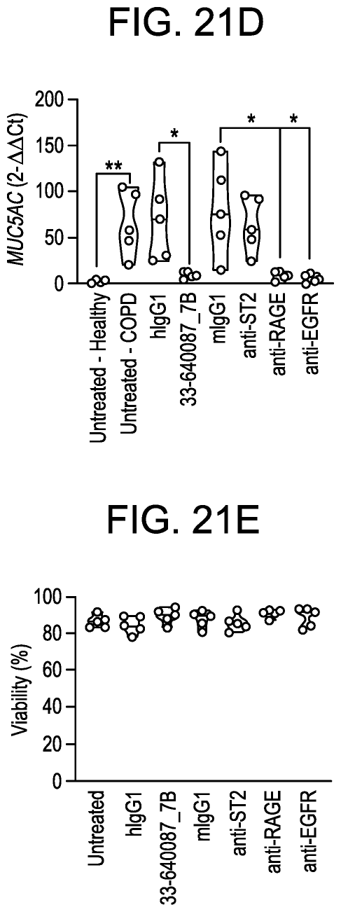

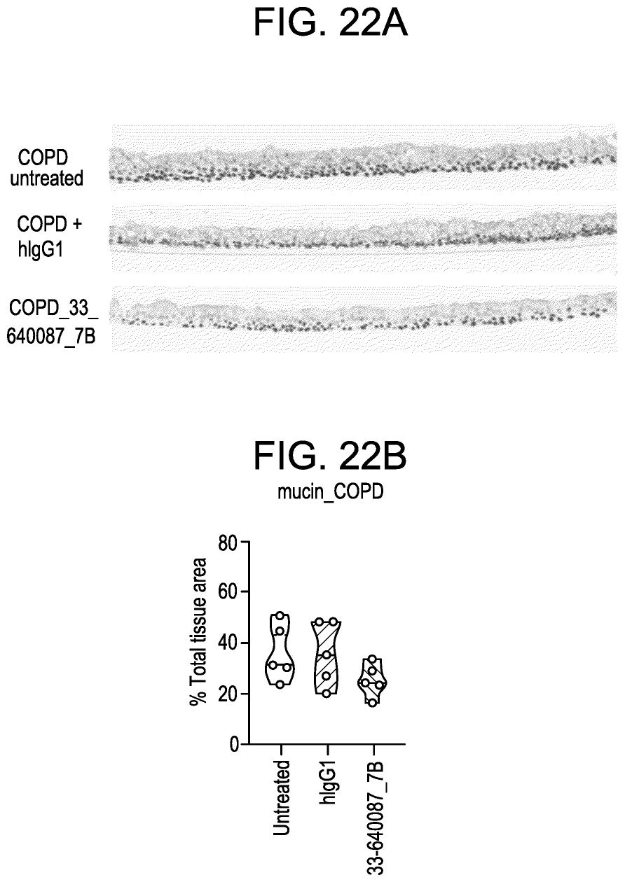

COPD ALI were cultured in the presence of anti-IL-33 (33-640087_7B), anti-RAGE or anti-EGFR neutralizing antibodies. All three treatments resulted in reduced MUC5AC+goblet cell numbers (FIG. 21A-D). No treatments affected the viability of the ALI cultures (FIG. 21E), confirming that the treatment phenomenon is not an artefact or a result of antibody toxicity. Consistent with previous results, anti-ST2 treatment did not result in reduction of goblet cell numbers, providing further evidence that this is a disease phenotype mediated directly by IL-33, principally ox-IL-33, through the oxIL-33-RAGE-EGFR pathway. The effect of anti-IL-33 antibody (33-640087_7B) on COPD ALI cultures was further confirmed in immunohistochemistry analysis where blockade of IL-33 resulted in decreased goblet cell numbers in pairwise analysis (FIGS. 22A and 22B). After treatment with anti-IL-33 antibody (33-640087_7B), the epithelium of the COPD ALI culture resembled healthy epithelium as shown in FIG. 20A.

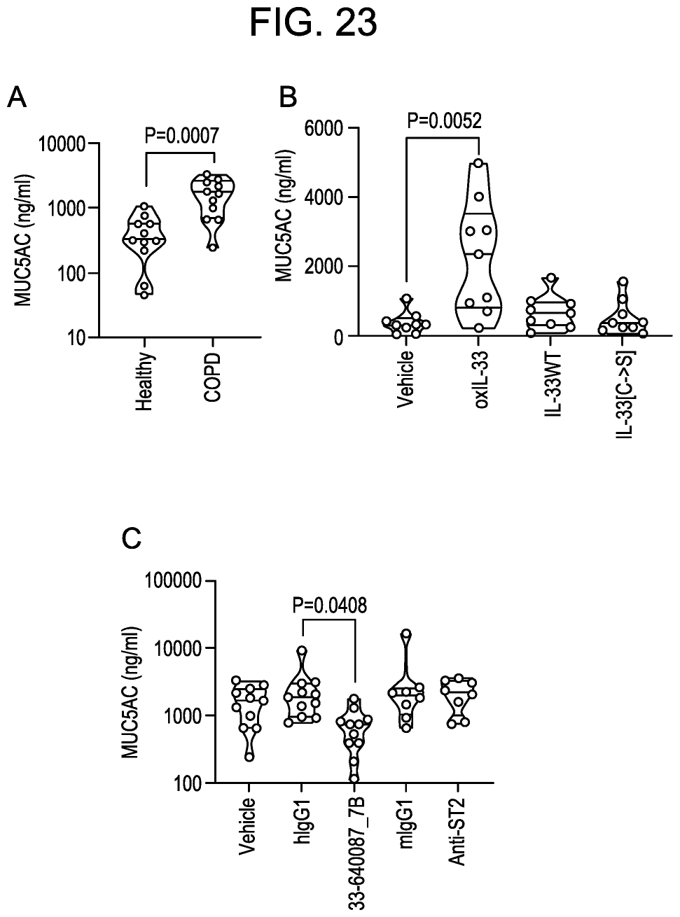

Lastly, MUC5AC and MUC5B released in the apical mucus from both healthy and COPD ALI cultures were measured using ELISAs. For quantification of mucin released from ALI cultures apical supernatants were analysed for levels of MUC5AC by immunoassay (Novus NBP2-76703) according to manufacturer's protocol. Samples were diluted 1:2000 in sample diluent and concentrations extrapolated from recombinant MUC5AC protein standard curves. As shown in FIG. 23, ALI cultures from COPD patients released increased levels of MUC5AC compared to ALI from healthy donors (FIG. 23A). Treatment with exogenous oxIL-33 resulted in increased mucins secretion from Healthy ALI cultures (FIG. 23B). In COPD ALI donors, the increased levels of mucins were reduced through blockade with anti-IL-33 (33_640087_7B) which inhibited MUC5AC protein levels released from ALI cultures, but not anti-ST2 or isotype mAb controls (FIG. 23C).

Overall this Example highlights a role for oxidised IL-33 in the dysregulation of epithelial cell differentiation in the lung. The results imply that, when uncontrolled, oxidised IL-33 may be responsible for the goblet cell hyperplasia and excessive mucus production seen in some phenotypes of COPD. Therefore, treatment with oxIL-33 signaling axis antagonists, such as anti-IL-33, anti-RAGE or anti-EGFR binding molecules, may be of great therapeutic benefit to COPD patients, by restoring normal epithelium physiology, for example, by decreasing goblet cell numbers and reducing of excessive mucus production.

Additional Sequences
Further to the sequences listed in Table 1,
we provide the following additional CDR
sequences
SEQ ID NO 37:
SYAMS
SEQ ID NO 38:
GISAIDQSTYYADSVKG
SEQ ID NO 39:
QKFMQLWGGGLRYPFGY
SEQ ID NO 40:
SGEGMGDKYAA
SEQ ID NO 41:
RDTKRPS
SEQ ID NO 42:
GVIQDNTGV
N terminal His10/Avitag/Factor Xa protease
cleavage site
SEQ ID NO 43:
MHHHHHHHHHHAAGLNDIFEAQKIEWHEAAIEGR
IL-33-01
SEQ ID NO 44:
SITGISPITEYLASLSTYNDQSITFALEDESYEIY
VEDLKKDEKKDKVLLSYYESQHPSNESGDGVDGKM
LMVTLSPTKDFWLHANNKEHSVELHKCEKPLPDQA
FFVLHNMHSNCVSFECKTDPGVFIGVKDNHLALIK
VDSSENLCTENILFKLSET
IL-33-16
SEQ ID NO 45:
SITGISPITEYLASLSTYNDQSITFALEDESYEIY
VEDLKKDEKKDKVLLSYYESQHPSNESGDGVDGKM
LMVTLSPTKDFWLHANNKEHSVELHKSEKPLPDQA
FFVLHNMHSNSVSFESKTDPGVFIGVKDNHL
ALIKVDSSENLSTENILFKLSET
Avitag sequence motif
SEQ ID NO 46:
GLNDIFEAQKIEWHE
gRNA vector targeting RAGE exon 3
SEQ ID NO 47:
TGAGGGGATTTTCCGGTGC
RAGE forward primer
SEQ ID NO 48:
gttgcagcctcccaacttc
RAGE reverse primer
SEQ ID NO 49:
aatgaggccagtggaagtca
Human ST2S (signal peptide underlined)
SEQ ID NO 50:
MGFWILAILTILMYSTAAKFSKQSWGLENEALIVR
CPRQGKPSYTVDWYYSQTNKSIPTQERNRVFASGQ
LLKFLPAAVADSGIYTCIVRSPTFNRTGYANVTIY
KKQSDCNVPDYLMYSTVSGSEKNSKIYCPTIDLYN
WTAPLEWFKNCQALQGSRYRAHKSFLVIDNVMTED
AGDYTCKFIHNENGANYSVTATRSFTVKDEQGFSL
FPVIGAPAQNEIKEVEIGKNANLTCSACFGKGTQF
LAAVLWQLNGTKITDFGEPRIQQEEGQNQSFSNGL
ACLDMVLRIADVKEEDLLLQYDCLALNLHGLRRHT
VRLSRKNPSKECF
Human ST2S-huIgGl Fc-His6 (signal
peptide underlined)
SEQ ID NO 51:
MPLLLLLPLLWAGALAKFSKQSWGLENEALIVRCP
RQGKPSYTVDWYYSQTNKSIPTQERNRVFASGQLL
KFLPAAVADSGIYTCIVRSPTFNRTGYANVTIYKK
QSDCNVPDYLMYSTVSGSEKNSKIYCPTIDLYNWT
APLEWFKNCQALQGSRYRAHKSFLVIDNVMTEDAG
DYTCKFIHNENGANYSVTATRSFTVKDEQGFSLFP
VIGAPAQNEIKEVEIGKNANLTCSACFGKGTQFLA
AVLWQLNGTKITDFGEPRIQQEEGQNQSFSNGLAC
LDMVLRIADVKEEDLLLQYDCLALNLHGLRRHTVR
LSRKNPSKECFEPKSCDKTHTCPPCPAPELLGGPS
VFLFPPKPKDTLMISRTPEVTCVVVDVSHEDPEVK
FNWYVDGVEVHNAKTKPREEQYNSTYRVVSVLTVL
HQDWLNGKEYKCKVSNKALPAPIEKTISKAKGQPR
EPQVYTLPPSREEMTKNQVSLTCLVKGFYPSDIAV
EWESNGQPENNYKTTPPVLDSDGSFFLYSKLTVDK
SRWQQGNVFSCSVMHEALHNHYTQKSLSLSPGKHH
HHHH
His10/Avitag human ASGPR ECD
(signal peptide underlined
and tags double underlined)
SEQ ID NO 52:
MPLLLLLPLLWAGALAHHHHHHHHHHggs
GLNDIFEAQKIEWHEGGSQNSQLQEELRGLRETFS
NFTASTEAQVKGLSTQGGNVGRKMKSLESQLEKQQ
KDLSEDHSSLLLHVKQFVSDLRSLSCQMAALQGNG
SERTCCPVNWVEHERSCYWFSRSGKAWADADNYCR
LEDAHLVVVTSWEEQKFVQHHIGPVNTWMGLHDQN
GPWKWVDGTDYETGFKNWRPEQPDDWYGHGLGGGE
DCAHFTDDGRWNDDVCQRPYRWVCETELDKASQEP
PLL

1. An IL-33 antagonist for use in the prevention or treatment of abnormal epithelium physiology by modulating or inhibiting a RAGE-EGFR mediated effect.

2. An IL-33 antagonist for use according to claim 1, wherein the abnormal epithelium physiology is abnormal mucociliary physiology, preferably abnormal mucociliary physiology of respiratory epithelium.

3. An IL-33 antagonist for use according to claim 2, wherein abnormal mucociliary physiology is selected from: abnormal mucus production; abnormal goblet cell differentiation, abnormal goblet cell proliferation; abnormal thickness of the epithelium; abnormal mucus clearance; and/or abnormal mucus composition.

4. An IL-33 antagonist for use according to claim 3, wherein abnormal mucus production comprises abnormal MUC5AC production; and/or wherein abnormal goblet cell differentiation comprises abnormal MUC5AC+goblet cell differentiation; and/or wherein abnormal goblet cell proliferation comprises abnormal MUC5AC+goblet cell proliferation; and/or wherein abnormal thickness of the epithelium comprises an abnormal amount of MUC5AC⁺ goblet cells in the total tissue area of the epithelium.

5. An IL-33 antagonist for use according to claim 2, 3 or 4, wherein abnormal mucociliary physiology comprises: increased mucus production; increased goblet cell differentiation; increased goblet cell proliferation; increased thickness of the epithelium; and/or decreased mucus clearance.

6. An IL-33 antagonist for use according to claim 5, wherein increased mucus production comprises increased MUC5AC production; and/or wherein increased goblet cell differentiation comprises increased MUC5AC+goblet cell differentiation; and/or wherein increased goblet cell proliferation comprises increased MUC5AC+goblet cell proliferation; and/or wherein increased thickness of the epithelium comprises an increased amount of MUC5AC⁺ goblet cells in the total tissue area of the epithelium.

7. An IL-33 antagonist for use according to claim 3, wherein abnormal mucus composition comprises an increase or a decrease in the ratio of the different mucus compounds contained in mucus; an increase or decrease in one or more mucus compounds; and/or an increase or decrease in the concentration or thickness of mucus.

8. An IL-33 antagonist for use according to claim 7, wherein abnormal mucus composition comprises an increase in the ratio of MUC5AC:MUC5B; and/or wherein abnormal mucus composition comprises an increase in MUC5AC contained in mucus; and/or wherein abnormal mucus composition comprises an increase in thickness of mucus.

9. An IL-33 antagonist for use according to claim 1, wherein the abnormal epithelium physiology is abnormal epithelium remodeling.

10. An IL-33 antagonist for use according to any preceding claim, wherein the abnormal epithelium physiology is of the respiratory tract, preferably abnormal mucociliary physiology of the respiratory tract.

11. An IL-33 antagonist for use according to claim 10, wherein the respiratory tract is the lower respiratory tract, preferably the bronchi.

12. An IL-33 antagonist for use in the prevention or treatment of an EGFR-mediated disease.

13. An IL-33 antagonist for use according to claim 12, wherein the EGFR-mediated disease is a RAGE-EGFR mediated disease.

14. An IL-33 antagonist for use according to claim 12 or 13 wherein the EGFR mediated disease is characterized by aberrant EGFR activity.

15. An IL-33 antagonist for use according to any of claims 12-14 wherein the EGFR mediated disease is characterized by abnormal epithelium physiology.

16. An IL-33 antagonist for use in the prevention or treatment of a disease by improving epithelium physiology.

17. An IL-33 antagonist for use in the prevention or treatment of a disease by inhibiting an EGFR-mediated effect.

18. An IL-33 antagonist for use according to claim 17, wherein the EGFR-mediated effect is EGFR signalling.

19. An IL-33 antagonist for use according to claim 17 or 18, wherein the EGFR-mediated effect is a RAGE-EGFR-mediated effect.

20. An IL-33 antagonist for use according to any of claims 17-19, wherein the EGFR-mediated effect is RAGE-EGFR-mediated signalling.

21. An IL-33 antagonist for use according to any of claims 16-20, wherein the disease is a respiratory disease.

22. An IL-33 antagonist for use according to claim 21 wherein the respiratory disease is characterised by abnormal epithelium physiology and/or aberrant EGFR activity.

23. An IL-33 antagonist for use according to claim 21 or 22, wherein the respiratory disease is a lower respiratory disease, preferably a respiratory disease of the bronchi.

24. An IL-33 antagonist for use according to any of claims 21-23, wherein the respiratory disease is selected from: COPD, bronchitis, emphysema, bronchiectasis, such as CF-bronchiectasis or -CF-bronchiectasis, asthma or asthma and COPD overlap (ACO).

25. An IL-33 antagonist for use according to any of claims 21-24, wherein the respiratory disease is COPD, preferably bronchitic COPD.

26. An IL-33 antagonist for use according to any of claims 21-24, wherein the respiratory disease is asthma, preferably bronchitic asthma.

27. An IL-33 antagonist for use according to any of claims 16-26, wherein the prevention or treatment improves mucus clearance.

28. An IL-33 antagonist for use according to any of claims 16-27, wherein the prevention or treatment inhibits or reduces abnormal mucus production.

29. An IL-33 antagonist for use according to claim 28, wherein the prevention or treatment inhibits or reduces MUC5AC production.

30. An IL-33 antagonist for use according to any of claims 16-29, wherein the prevention or treatment inhibits an abnormal mucus composition.

31. An IL-33 antagonist for use according to claim 30, wherein the prevention or treatment inhibits or reduces the ratio of MUC5AC:MUC5B; and/or wherein the prevention or treatment inhibits or reduces MUC5AC in mucus; and/or wherein the prevention or treatment reduces the thickness of mucus.

32. An IL-33 antagonist for use according to any of claims 16-31, wherein the prevention or treatment inhibits abnormal epithelium remodelling.

33. An IL-33 antagonist for use according to any of claims 16-32, wherein the prevention or treatment inhibits abnormal goblet cell differentiation or proliferation.

34. An IL-33 antagonist for use according to claim 33, wherein the abnormal goblet cell differentiation or proliferation is abnormal MUC5AC⁺ goblet cell differentiation or proliferation.

35. An IL-33 antagonist for use according to any of claims 16-34, wherein the prevention or treatment reduces the thickness of the respiratory epithelium.

36. An IL-33 antagonist for use according to claim 35, wherein the prevention or treatment reduces the amount of MUC5AC⁺ goblet cells in the total tissue area of the epithelium.

37. An IL-33 antagonist for use according to any preceding claim, wherein the IL-33 antagonist inhibits the activity of oxidised IL-33.

38. An IL-33 antagonist for use according to any preceding claim, wherein the IL-33 antagonist prevents binding of oxidised IL-33 to RAGE, thereby inhibiting RAGE-EGFR signalling.

39. An IL-33 antagonist for use according to any preceding claim, wherein the IL-33 antagonist down-regulates or inhibits RAGE-EGFR dependent signalling and/or RAGE-EGFR mediated effects.

40. An IL-33 antagonist for use according to any preceding claim, wherein the IL-33 antagonist is a binding molecule or fragment thereof which binds to IL-33, preferably which binds to reduced IL-33 or oxidised IL-33, preferably which binds to reduced IL-33.

41. An IL-33 antagonist for use according to any preceding claim, wherein the IL-33 antagonist is an antibody or antigen-binding fragment thereof, preferably an anti-IL-33 antibody or antigen-binding fragment thereof, preferably an anti-reduced-IL-33 antibody or antigen-binding fragment thereof.

42. An IL-33 antagonist for use according to any preceding claim, wherein the IL-33 antagonist is a binding molecule which comprises complementarity determining regions (CDRs) of a variable heavy domain (VH) and a variable light domain (VL) pair selected from Table 1.

43. An IL-33 antagonist for use according to any preceding claim, wherein the IL-33 antagonist is a binding molecule which comprises a variable heavy domain (VH) and variable light domain (VL) pair selected from Table 1.

44. An IL-33 antagonist for use according to any preceding claim, wherein the IL-33 antagonist is a binding molecule which comprises a VHCDR1 having the sequence of SEQ ID NO: 37, a VHCDR2 having the sequence of SEQ ID NO: 38, a VHCDR3 having the sequence of SEQ ID NO: 39, a VLCDR1 having the sequence of SEQ ID NO: 40, a VLCDR2 having the sequence of SEQ ID NO: 41, and a VLCDR3 having the sequence of SEQ ID NO: 42.

45. A method for the prevention or treatment of abnormal epithelium physiology in a subject, by administering a therapeutically effective amount of an IL-33 antagonist to modulate or inhibit a RAGE-EGFR mediated effect.

46. The method of claim 45, wherein the abnormal epithelium physiology is abnormal mucociliary physiology, preferably abnormal mucociliary physiology of respiratory epithelium.

47. The method of claim 46, wherein abnormal mucociliary physiology is selected from: abnormal mucus production; abnormal goblet cell differentiation; abnormal goblet cell proliferation; abnormal thickness of the epithelium; abnormal mucus clearance; and/or abnormal mucus composition.

48. The method of claim 47, wherein abnormal mucus production comprises abnormal MUC5AC production; and/or wherein abnormal goblet cell differentiation comprises abnormal MUC5AC+goblet cell differentiation; and/or wherein abnormal goblet cell proliferation comprises abnormal MUC5AC+goblet cell proliferation; and/or wherein abnormal thickness of the epithelium comprises an abnormal amount of MUC5AC⁺ goblet cells in the total tissue area of the epithelium.

49. The method of claim 47 or 48, wherein abnormal mucociliary physiology comprises: increased mucus production; increased goblet cell differentiation; increased goblet cell proliferation; increased thickness of the epithelium; and/or decreased mucus clearance.

50. The method of claim 49, wherein increased mucus production comprises increased MUC5AC production; and/or wherein increased goblet cell differentiation comprises increased MUC5AC+goblet cell differentiation; and/or wherein increased goblet cell proliferation comprises increased MUC5AC+goblet cell proliferation; and/or wherein increased thickness of the epithelium comprises an increased amount of MUC5AC⁺ goblet cells in the total tissue area of the epithelium.

51. The method of claim 47, wherein abnormal mucus composition comprises an increase or a decrease in the ratio of the different mucus compounds contained in mucus; an increase or decrease in one or more mucus compounds; and/or an increase or decrease in the concentration or thickness of mucus.

52. The method of claim 51, wherein abnormal mucus composition comprises an increase in the ratio of MUC5AC:MUC5B; and/or wherein abnormal mucus composition comprises an increase in MUC5AC contained in mucus; and/or wherein abnormal mucus composition comprises an increase in thickness of mucus.

53. The method of claim 45, wherein the abnormal epithelium physiology is abnormal epithelium remodelling.

54. The method of any of claims 45-53, wherein the abnormal epithelium physiology is of the respiratory tract, preferably abnormal mucociliary physiology of the respiratory tract.

55. The method of claim 54, wherein the respiratory tract is the lower respiratory tract, preferably the bronchi.

56. The method for the prevention or treatment of an EGFR-mediated disease, by administering a therapeutically effective amount of an IL-33 antagonist.

57. The method of claim 56, wherein the EGFR-mediated disease is a RAGE-EGFR mediated disease.

58. The method of claim 56 or 57, wherein the EGFR mediated disease is characterised by aberrant EGFR activity.

59. The method of any of claims 56-58 wherein the EGFR mediated disease is characterised by abnormal epithelium physiology.

60. A method for the prevention or treatment of a disease, by administering a therapeutically effective amount of an IL-33 antagonist to improve epithelium physiology.

61. A method for the prevention or treatment of a disease, by administering a therapeutically effective amount of an IL-33 antagonist to inhibit an EGFR-mediated effect.

62. The method of claim 61, wherein the EGFR-mediated effect is EGFR signalling.

63. The method of claim 61 or 62, wherein the EGFR-mediated effect is a RAGE-EGFR-mediated effect.

64. The method of any of claims 61-63, wherein the EGFR-mediated effect is RAGE-EGFR-mediated signalling.

65. The method of any of claims 60-64, wherein the disease is a respiratory disease.

66. The method of claim 65, wherein the respiratory disease is characterised by abnormal epithelium physiology and/or aberrant EGFR activity.

67. The method of claim 65 or 66, wherein the respiratory disease is a lower respiratory disease, preferably a respiratory disease of the bronchi.

68. The method of any of claims 65-67, wherein the respiratory disease is selected from: COPD, bronchitis, emphysema, bronchiectasis, such as CF-bronchiectasis or -CF-bronchiectasis, asthma or asthma and COPD overlap (ACO).

69. The method of any of claims 65-68, wherein the respiratory disease is COPD, preferably bronchitic COPD.

70. The method of any of claims 65-69, wherein the respiratory disease is asthma, preferably bronchitic asthma.

71. The method of any of claims 45-70, wherein the method improves mucus clearance.

72. The method of any of claims 45-71, wherein the method inhibits or reduces abnormal mucus production.

73. The method of claim 72, wherein the abnormal mucus production is an increase in MUC5AC production.

74. The method of any of claims 45-73, wherein the method inhibits an abnormal mucus composition.

75. The method of claim 74, wherein the method inhibits or reduces the ratio of MUC5AC:MUC5B; and/or wherein the method inhibits or reduces MUC5AC in mucus; and/or wherein the method reduces the thickness of mucus.

76. The method of any of claims 45-75, wherein the method inhibits abnormal epithelium remodeling.

77. The method of any of claims 45-76, wherein the method inhibits or reduces abnormal goblet cell differentiation or proliferation.

78. The method of claim 77, wherein the method inhibits or reduces abnormal MUC5AC⁺ goblet cell differentiation or proliferation.

79. The method of any of claims 45-78, wherein the method reduces the thickness of the respiratory epithelium.

80. The method of claim 79, wherein the method reduces the amount of MUC5AC⁺ goblet cells in the total tissue area of the respiratory epithelium.

81. The method of any of claims 45-80, wherein the IL-33 antagonist inhibits the activity of oxidised IL-33.

82. The method of any of claims 45-81, wherein the IL-33 antagonist prevents binding of oxidised IL-33 to RAGE, thereby inhibiting RAGE-EGFR signalling.

83. The method of any of claims 45-82, wherein the IL-33 antagonist down-regulates or inhibits RAGE-EGFR dependent signalling and/or RAGE-EGFR mediated effects.

84. The method of any of claims 45-83, wherein the IL-33 antagonist is a binding molecule or fragment thereof which binds to IL-33, preferably reduced IL-33 or oxidised IL-33, preferably reduced IL-33.

85. The method of any of claims 45-84, wherein the IL-33 antagonist is an antibody or antigen-binding fragment thereof, preferably an anti-IL-33 antibody or antigen-binding fragment thereof, preferably an anti-reduced-IL-33 antibody or antigen-binding fragment thereof.

86. The method of any of claims 45-85, wherein the IL-33 antagonist is a binding molecule which comprises complementarity determining regions (CDRs) of a variable heavy domain (VH) and a variable light domain (VL) pair selected from Table 1.

87. The method of any of claims 45-86, wherein the IL-33 antagonist is a binding molecule which comprises a variable heavy domain (VH) and variable light domain (VL) pair selected from Table 1.

88. The method of any of claims 45-87, wherein the IL-33 antagonist is a binding molecule which comprises a VHCDR1 having the sequence of SEQ ID NO: 37, a VHCDR2 having the sequence of SEQ ID NO: 38, a VHCDR3 having the sequence of SEQ ID NO: 39, a VLCDR1 having the sequence of SEQ ID NO: 40, a VLCDR2 having the sequence of SEQ ID NO: 41, and a VLCDR3 having the sequence of SEQ ID NO: 42.

89. The method of any of claims 45-88, or the IL-33 antagonist for use according to any of claims 1-44, wherein the IL-33 antagonist is an anti-IL33 antibody or antigen binding fragment thereof comprising a VH domain of the sequence of SEQ ID NO:1 and a VL domain of the sequence of SEQ ID NO:19.
